SEQUENCE LISTING (1 sequence)

SEQ ID NO: 1 - DNA, 28 bp, Artificial Sequence
  Synthetic
gtccaggaggcagctaatacttgttgcg

(19) United States
(12) Patent Application Publication
(10) Pub. No.: US 2019/0187031 A1
(43) Pub. Date: Jun. 20, 2019

(54) CONCENTRATION OF ANALYTES

(71) Applicant: THE REGENTS OF THE UNIVERSITY OF MICHIGAN, ANN ARBOR, MI (US)

(72) Inventors: ALEXANDER EDMUND JOHNSON-BUCK, Ann Arbor, MI (US); MUNEESH TEWARI, Ann Arbor, MI (US); NILS G. WALTER, Ann Arbor, MI (US)

(21) Appl. No.: 16/219,070

(22) Filed: Dec. 13, 2018

Related U.S. Application Data
(60) Provisional application No. 62/692,001, filed on Jun. 29, 2018.
(60) Provisional application No. 62/598,802, filed on Dec. 14, 2017.

(51) Int. Cl. G01N 1/40; C12Q 1/6806

(52) U.S. Cl. CPC G01N 1/405; C12Q 1/6806

(57) ABSTRACT

Provided herein is technology relating to detection of analytes and particularly, but not exclusively, to compositions, methods, and systems for concentrating an analyte at a surface, e.g., for imaging and detection of low-abundance analytes.

The present application claims priority to U.S. Provisional Patent Application Ser. No. 62/692,001, filed Jun. 29, 2018, and U.S. Provisional Patent Application Ser. No. 62/598,802, filed Dec. 14, 2017, each of which is incorporated herein by reference in its entirety.

### STATEMENT REGARDING FEDERALLY SPONSORED RESEARCH OR DEVELOPMENT

This invention was made with government support under W911NF-12-1-0420 awarded by the U.S. Army Research Laboratory's Army Research Office (ARO), and GM062357 awarded by the National Institutes of Health. The government has certain rights in the invention.

### FIELD

Provided herein is technology relating to detection of analytes and particularly, but not exclusively, to compositions, methods, and systems for concentrating an analyte at a surface, e.g., for imaging and detection of low-abundance analytes.

### BACKGROUND

Some molecular diagnostic technologies permit the identification of very low-abundance biomarkers, such as a small population of tumor-derived DNA in a blood sample comprising a large excess of non-tumor DNA or a rare genetic allele commingled in a mixed sample with other, more abundant or wild-type alleles. In some samples, these informative biomarkers are present at abundances as low as a few (e.g., 1-1000) copies per milliliter of biofluid (blood, plasma, urine, etc.) Analyzing biomarkers present at low concentrations (e.g., less than 1 femtomolar and/or approximately 1-1000 molecules per milliliter of a biofluid) poses sensitivity challenges for many technologies, especially for technologies involving surface immobilization (e.g., microarrays (see, e.g., Schulze & Downward (2001) “Navigating gene expression using microarrays—a technology review” Nat. Cell Biol. Lond. 3: E190-95), direct digital detection of nucleic acids using target-specific, color-coded probe pairs (e.g., the NANOSTRING NCOUNTER, see, e.g., Kulkarni (2001) “Digital Multiplexed Gene Expression Analysis Using the NanoString nCounter System” in Current Protocols in Molecular Biology (John Wiley & Sons, Inc.)), and most next-generation sequencing platforms (see, e.g., Metzker (2010) “Sequencing technologies—the next generation” Nat. Rev. Genet. 11: 31-46; Eid et al. (2009) “Real-Time DNA Sequencing from Single Polymerase Molecules” Science 323: 133-38).

Some existing technologies use surface capture or analyte extraction to increase the concentration of low-abundance analytes (e.g., surface capture for next-generation sequencing, droplet generation for droplet digital PCR, etc.). However, these technologies are costly and/or time consuming (e.g., requiring several hours), especially when processing large volumes of biofluids for analysis. Other technologies have used nucleic acid amplification techniques such as PCR to increase the amount of a target nucleic acid in a sample. However, nucleic acid amplification introduces both chemical damage and copying errors into amplified products (see, e.g., Potapov & Ong (2017) “Examining Sources of Error in PCR by Single-Molecule Sequencing” PLOS ONE 12: e0169774). While these errors can in principle be identified and removed with the addition of unique molecular identifiers (or barcode sequences) accompanied by high read depth (Schmitt et al. (2012) “Detection of ultra-rare mutations by next-generation sequencing” Proc. Natl. Acad. Sci 109: 14508-13), this strategy adds significantly to the cost and analytical complexity of an assay. Furthermore, while analytes such as DNA and RNA sequences can be amplified by PCR, other biomarkers (e.g., methylated and other modified nucleobases, peptides, proteins, complex carbohydrates, glycoproteins, lipids, post-translational modifications, metabolites, etc) cannot be copied by PCR or any similar process.

### SUMMARY

Accordingly, provided herein is a technology for concentrating analytes (e.g., biomolecules (e.g., nucleic acids (e.g., DNA, RNA, methylated and other modified or non-naturally occurring nucleobases), polypeptides (e.g., peptides, proteins, glycoproteins), carbohydrates, lipids, post-translational modifications, amino acids, metabolites), small molecules, etc.). In some embodiments, the technology relates to using aqueous two-phase systems (ATPS) to concentrate analytes at a surface. In some embodiments, the technology relates to use of electrophoretic concentration of analytes at a surface. In some embodiments, concentration of analytes at a surface is followed by surface capture of analytes (e.g., immobilization of analytes at the surface). In some embodiments, concentration of analytes at a surface and, optionally, surface capture of analytes at a surface is followed by analysis of the analytes.

During the development of embodiments of the technology described herein, experiments using ATPS indicated that embodiments of the technology provided greater than an 80-fold increase in the surface density of captured tumor DNA sequences using an ATPS comprising poly(ethylene glycol) (PEG) 3350, sodium citrate, and sodium chloride. The increase in the surface density of captured analyte resulted in greater than an 80-fold increase in the sensitivity of subsequent detection. Further, during the development of embodiments of the technology provided herein, experiments using electrophoretic concentration indicated that electrophoretic concentration of analytes (e.g., charged analytes (e.g., negatively charged, positively charged)) provided at least a 50-fold to 100-fold increase in concentration of the analyte. In some embodiments, the technology comprises use of an ATPS as described in U.S. provisional patent application Ser. No. 62/598,802, filed Dec. 14, 2017, which is incorporated herein by reference in its entirety.

An aqueous two-phase system ATPS (see, e.g., Iqbal et al. (2016) “Aqueous two-phase system (ATPS): an overview and advances in its applications” Biol. Proced. Online 18, incorporated herein by reference in its entirety) arises when two water-soluble components—e.g., two polymers or a polymer and a salt—exceed a threshold concentration and separate into two aqueous phases with distinct compositions. Several types of ATPS selectively partition biomolecules, including DNA and proteins, into one of the two phases. In some ATPS systems, the concentration of a biomolecule in one of the phases is greater than 1000 times the concentration of the biomolecule in the other phase (Alberts (1967) “Efficient Separation of Single-Stranded and Double-Stranded Deoxyribonucleic Acid in a Dextran-Polyethylene Glycol Two-Phase System” Biochemistry (Mosc.) 6: 2527-32, incorporated herein by reference in its entirety). When one of the two ATPS components is present in excess relative to the other, the two resulting phases can have unequal volumes at equilibrium, making it possible to concentrate a biomolecule of interest in the smaller phase (see, e.g., Iqbal, supra). In some embodiments of the technology provided herein, an ATPS system is used to concentrate very dilute solutions of DNA over a small region of an imaging surface for rapid and efficient capture of the analyte (see, e.g., FIG. 1a). In some embodiments, methods comprise heating a sample (e.g., to denature a biomolecule (e.g., nucleic acid (e.g., RNA, DNA), protein, or other molecule, biomolecule, and/or molecular complex), mixing the sample vigorously with ATPS-forming components, then centrifuging the ATPS comprising the sample (e.g., comprising an analyte from the sample) briefly to position the analyte-rich lower phase near the imaging surface. In particular, embodiments of methods comprise heating a DNA sample (e.g., to denature the DNA), mixing the DNA sample vigorously with ATPS-forming components, then centrifuging the ATPS and DNA briefly to position the DNA-rich lower phase near the imaging surface. The technology is straightforward and is completed in 1-10 minutes.

Electrophoretic capture (e.g., of an analyte) comprises use of a voltage to move an analyte (e.g., a charged analyte (e.g., positively charged, negatively charged)) to a surface and/or to concentrate an analyte (e.g., a charged analyte (e.g., positively charged, negatively charged)) at the surface (e.g., to move an analyte to contact a surface). In some embodiments, the analyte is concentrated at particular sites on a surface (e.g., at particular, defined, and/or addressable sites on the surface). In some embodiments, the analyte is delivered to the surface and allowed to diffuse over the surface to provide a homogenous distribution of the analyte over the surface. In some embodiments, the analyte is captured at the surface (e.g., as described herein (e.g., by a capture probe)) after delivery of the analyte to the surface and/or after contact of the analyte to the surface. In some embodiments, electrophoretic capture of an analyte comprises use of an electrophoretic sample cell (e.g., as described herein) comprising an inner chamber, an outer chamber, and a voltage applied across the inner chamber and outer chamber. In some embodiments, the electrophoretic sample cell comprises a porous material that allows passage of current between the inner chamber and outer chamber and minimizes and/or eliminates flow of analyte between the inner chamber and outer chamber.

Accordingly, in some embodiments the technology provides a method for analysis of an analyte on a substrate. For example, in some embodiments, the method comprises providing an aqueous two-phase system (ATPS) comprising an analyte; concentrating the analyte into a first phase of the ATPS; and contacting said first phase to a substrate. In some embodiments, the methods further comprise separating said first phase from a second phase, e.g., by centrifugation or gravity. In some embodiments, the concentration of the analyte in the first phase is more than the concentration of the analyte in the second phase (e.g., at least approximately 2, 3, 4, 5, 6, 7, 8, 9, 10, 11, 12, 13, 14, 15, 16, 17, 18, 19, or 20 times the concentration of the analyte in the second phase). In some embodiments, the volume of the second phase is more than the volume of the first phase (e.g., at least approximately 2, 3, 4, 5, 6, 7, 8, 9, 10, 11, 12, 13, 14, 15, 16, 17, 18, 19, or 20 times the volume of the first phase). In some embodiments, contacting the first phase to the substrate occurs for approximately more than 20 seconds (e.g., more than approximately 20, 30, 40, 50, 60, 70, 80, 90, 100, 110, or 120 seconds). In some embodiments, contacting the first phase to the substrate occurs for approximately more than 60 seconds.

In some embodiments, providing said ATPS comprises providing a first composition and a second composition that form an ATPS upon mixing. In some embodiments, providing said ATPS comprises providing a mixture of a first composition and a second composition that form an ATPS upon adding an aqueous sample comprising said analyte to said mixture. In some embodiments, providing said ATPS comprises mixing a first composition and a second composition to form a mixture that forms an ATPS. In some embodiments, providing said ATPS comprises mixing a first composition and a second composition and adding an aqueous sample comprising said analyte to said mixture. In some embodiments, the mixture provided is a solid, a neat mixture, or comprises solid and neat components. In some embodiments, the ATPS comprises polyethylene glycol (PEG) and citrate. In some embodiments, the ATPS comprises (PEG), citrate, and a salt (e.g., sodium chloride). In some embodiments, the first phase of the ATPS (e.g., the phase comprising the concentrated analyte) comprises citrate.

In some embodiments, methods comprise heating said sample comprising said analyte and/or heating said ATPS comprising said analyte. In some embodiments, the analyte is a nucleic acid and methods comprise denaturing said nucleic acid.

In some embodiments, providing said aqueous two-phase system (ATPS) comprising an analyte comprises adding an aqueous sample comprising said analyte to a first composition and a second composition that form an ATPS. In some embodiments, the analyte is denatured prior to adding said aqueous sample comprising said analyte to said first composition and said second composition that form the ATPS. In some embodiments, the analyte is denatured by heating. And, in some embodiments, the analyte is a nucleic acid.

In some embodiments, an ATPS comprises PEG, citrate, and a salt, e.g., in some embodiments the mass fraction of PEG in the mixture is greater than 30%, the mass fraction of citrate in the mixture is less than 2.5%, and the concentration of the salt is greater than 0.25 M. In some embodiments, the molecular weight of said PEG is between 600 and 6000 Da. In some embodiments, citrate is present as citric acid and sodium citrate in a ratio to provide a pH between 5.5 and 8.5.

In some embodiments related to ATPS technologies, the ATPS further comprises an enzyme, a detergent, a chaotropic agent, and/or an oligonucleotide.

In some embodiments, the technology relates to the use of an electrophoretic cell to move and/or concentrate analytes at a surface. For instance, some embodiments provide a method for analysis of an analyte on a substrate, e.g., a method comprising providing an electrophoretic cell comprising an inner chamber comprising a solution comprising an analyte and an outer chamber comprising a solution comprising an electrolyte; providing a voltage across the inner chamber and the outer chamber; and contacting the electrophoretic cell to a substrate. In some embodiments, the methods comprise providing a porous base for the outer chamber. In some embodiments, said porous base comprises a material (e.g., a polymer) through which current flows and through which flow of analyte is minimized and/or eliminated. In some embodiments, the flow of analyte through the porous base is inhibited on the basis of the molecular weight and/or hydrodynamic radius of the analyte, e.g., in some embodiments, the molecular weight and/or hydrodynamic radius of the analyte exceeds a threshold value that does not flow through the porous base.

In some embodiments, said voltage is provided by an alternating current source. In some embodiments, said voltage is provided by a direct current source. In some embodiments, said voltage varies with a period of approximately 1 minute (e.g., 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 minutes). In some embodiments, said voltage varies between 0 and approximately 50 to 120 V (e.g., 0, 5, 10, 15, 20, 25, 30, 35, 40, 45, 50, 55, 60, 65, 70, 75, 80, 85, 90, 95, 100, 105, 110, 115, or 120 V).

In some embodiments, methods comprising use of an electrophoretic cell further comprises capturing the analyte by a capture probe immobilized on said substrate. In some embodiments, methods further comprise detecting the analyte on the substrate, e.g., detecting the analyte on the substrate by analyzing the kinetics of association and dissociation of a detectably labeled query probe to the analyte (e.g., by SiMREPS). In some embodiments, methods comprise detecting the analyte on the substrate with single-molecule sensitivity.

In some embodiments, the capture probe comprises a nucleic acid, a polypeptide, a sugar, or a lipid. In some embodiments, the query probe comprises a nucleic acid, a polypeptide, a sugar, or a lipid. In some embodiments, the analyte comprises a nucleic acid, a polypeptide, a metabolite, a lipid, a small molecule, or a carbohydrate.

Further embodiments comprise providing a sample comprising said analyte. In some embodiments, said sample is a biological sample. In some embodiments, said sample is a biofluid. In some embodiments, said solution comprising said analyte is contacted to a substrate over an area of from 0.01 to 100 mm² (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mm²). In some embodiments, said inner chamber comprises an opening having a width of approximately 0.01 to 10 mm (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, or 10 mm).

In some embodiments, said analyte is a nucleic acid. For instance, in some embodiments said analyte is a nucleic acid comprising fewer than 200 basepairs or nucleotides. In some embodiments, said analyte is a nucleic acid comprising fewer than 40 basepairs or nucleotides. In some embodiments, said analyte is a single-stranded nucleic acid. In some embodiments, said analyte is a double-stranded nucleic acid. In particular embodiments, said sample comprises a cell, a single cell, at least 1 cell, at least 10 cells, at least 100 cells, at least 1000 cells, or a cluster of cells; a biofluid; a cell lysate, a lysate from a cell, a lysate from a single cell, a lysate from at least 1 cell, a lysate from at least 10 cells, a lysate from at least 100 cells, a lysate from at least 1000 cells, or a lysate from a cluster of cells; a subcellular compartment, a single subcellular compartment, at least 1 subcellular compartment, at least 10 subcellular compartments, at least 100 subcellular compartments, at least 1000 subcellular compartments; and/or a lysate of a subcellular compartment, a lysate of a single subcellular compartment, a lysate of at least 1 subcellular compartment, a lysate of at least 10 subcellular compartments, a lysate of at least 100 subcellular compartments, or a lysate of at least 1000 subcellular compartments.

In related embodiments, the technology provides a system for detecting an analyte. For example, embodiments of systems comprise an electrophoretic cell comprising an inner chamber capable of holding a solution comprising an analyte and an outer chamber capable of holding a solution comprising an electrolyte; a substrate for capturing said analyte; a voltage source; and a system configured to detect said analyte on the substrate by analyzing the kinetics of association and dissociation of a detectably labeled query probe to said analyte. In some embodiments, said substrate comprises an immobilized capture probe. In some embodiments, the query probe is a fluorescently labeled nucleic acid that associates repeatedly with the analyte with a kinetic rate constant k_{off} that is greater than 0.1 min⁻¹ and/or a kinetic rate constant kₒₙ that is greater than 0.1 min⁻¹. In some embodiments, said system is configured to detect said analyte comprises a fluorescence detector. In some embodiments, said system configured to detect said analyte comprises a software component configured to fit a two-state model to query probe binding data.

As described herein, the technology relates to use of an ATPS or an electrophoretic cell to concentrate an analyte at a surface. In some embodiments, the technology provides use of an ATPS or an electrophoretic cell to concentrate an analyte at a surface to detect said analyte on the substrate by analyzing the kinetics of association and dissociation of a detectably labeled query probe to said analyte. Some embodiments relate to use of an ATPS or an electrophoretic cell to concentrate an analyte at a surface, e.g., to quantify said analyte. In some embodiments, use of an ATPS or an electrophoretic cell to concentrate an analyte at a surface is used to concentrate an analyte that comprises a nucleic acid, a polypeptide, a metabolite, a lipid, a small molecule, or a carbohydrate.

In some embodiments, the methods further comprise capturing the analyte at the substrate surface (e.g., by a capture probe immobilized on said substrate). In some embodiments, methods further comprise detecting the analyte on the substrate (e.g., detecting the analyte on the substrate by analyzing the kinetics of association and dissociation of a detectably labeled query probe to the analyte). In some embodiments, methods comprise characterizing the analyte on the substrate, quantifying the analyte on the substrate, and/or imaging the analyte on the substrate. In some embodiments, the analyte is detected, characterized, quantified, and/or imaged with single-molecule sensitivity.

The technology is not limited in the capture probe. For example, in some embodiments, the capture probe comprises a nucleic acid, a polypeptide, a sugar, or a lipid. The technology is not limited in the query probe. For example, in some embodiments, the query probe comprises a nucleic acid, a polypeptide, a sugar, or a lipid. The technology is not limited in the analyte. For example, in some embodiments the analyte comprises a nucleic acid, a polypeptide, a metabolite, a lipid, a small molecule, or a carbohydrate. In some embodiments, the analyte is a nucleic acid (e.g., a nucleic acid comprising fewer than 200 basepairs or nucleotides (e.g., fewer than 200, 195, 190, 185, 180, 185, 180, 175, 170, 165, 160, 155, 150, 145, 140, 135, 130, 125, 120, 115, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 basepairs or nucleotides). In some embodiments, the analyte is a nucleic acid comprising fewer than 40 basepairs or nucleotides (e.g., fewer than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 basepairs or nucleotides). In some embodiments, the analyte is a single-stranded nucleic acid. In some embodiments, the analyte is a double-stranded nucleic acid. Further descriptions of capture probes, query probes, and analytes are provided elsewhere herein.

Some embodiments further comprise providing a sample comprising an analyte. The technology is not limited in the sample that comprises the analyte. For example, in some embodiments, the sample is or comprises a biological sample and/or a biofluid. For example, in some embodiments, the sample comprises a cell, a single cell, at least 1 cell, at least 10 cells, at least 100 cells, at least 1000 cells, or a cluster of cells; a biofluid; a cell lysate, a lysate from a cell, a lysate from a single cell, a lysate from at least 1 cell, a lysate from at least 10 cells, a lysate from at least 100 cells, a lysate from at least 1000 cells, or a lysate from a cluster of cells; a subcellular compartment, a single subcellular compartment, at least 1 subcellular compartment, at least 10 subcellular compartments, at least 100 subcellular compartments, at least 1000 subcellular compartments; and/or a lysate of a subcellular compartment, a lysate of a single subcellular compartment, a lysate of at least 1 subcellular compartment, a lysate of at least 10 subcellular compartments, a lysate of at least 100 subcellular compartments, or a lysate of at least 1000 subcellular compartments. Further description of samples is provided elsewhere herein.

In some embodiments, the technology relates to delivering an analyte to a substrate (e.g., to concentrate the analyte at the substrate surface and/or to immobilize the analyte at the substrate surface). Further, in some embodiments, the technology relates to improving the sensitivity of analyte detection by increasing the concentration of analyte on a substrate analyzed for detection of the analyte. Accordingly, in some embodiments relating to an ATPS, the first phase is contacted to a substrate over a restricted area of from 0.01 to 100 mm² (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mm²). In some embodiments, contacting the first phase to the substrate comprises use of a sample well comprising a restricted opening having an area of from 0.01 to 100 mm² (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mm²). Further, in some embodiments relating to use of an electrophoretic cell, the sample comprising the analyte is contacted to a substrate over a restricted area of from 0.01 to 100 mm² (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mm²) In some embodiments, contacting the sample comprising the analyte to the substrate comprises use of an electrophoretic cell comprising an inner chamber comprising a restricted opening having an area of from 0.01 to 100 mm² (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mm²).

In some embodiments, the methods further comprise adding an oligonucleotide to minimize and/or eliminate hybridization and/or formation of secondary structure by said nucleic acid. In some embodiments, an enzyme is added prior to denaturing said analyte. In some embodiments, a proteinase and/or a lipase is added prior to denaturing said analyte. In some embodiments, a detergent and/or chaotropic agent is added prior to denaturing said analyte.

Additional embodiments of the technology relate to a system for detecting an analyte. For example, in some embodiments the technology provides a system comprising an aqueous two-phase system (ATPS) for concentrating an analyte in a first phase of the ATPS; a substrate for capturing said analyte from said first phase of the ATPS; and a system configured to detect said analyte on the substrate by analyzing the kinetics of association and dissociation of a detectably labeled query probe to said analyte. In some embodiments, the substrate comprises an immobilized capture probe. In some embodiments, the query probe is a fluorescently labeled nucleic acid that associates repeatedly with the analyte with a kinetic rate constant k_{off} that is greater than 0.1 min⁻¹ and/or a kinetic rate constant kₒₙ that is greater than 0.1 min⁻¹. In some embodiments, the system configured to detect said analyte comprises a fluorescence detector. In some embodiments, the system is configured to detect said analyte comprises a software component configured to fit a two-state model to query probe binding data. In some embodiments, the system further comprises a centrifuge. In some embodiments, the system further comprises a sample well comprising a restricted opening having an area of 0.01 to 10 mm (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, or 10 mm). These various components of systems (e.g., ATPS, computers, sample wells, query probes, capture probes, substrates, and aspects of kinetic detection) are more fully described throughout the application, the relevant portions of which are incorporated here by reference.

Some embodiments relate to additional system embodiments for detecting an analyte. For example, in some embodiments the technology provides a system comprising an electrophoretic concentration cell (e.g., comprising an inner chamber capable of holding a solution comprising an analyte and an outer chamber capable of holding a buffer); a substrate for capturing said analyte from said inner chamber; and a system configured to detect said analyte on the substrate by analyzing the kinetics of association and dissociation of a detectably labeled query probe to said analyte. In some embodiments, systems comprise a porous material (e.g., a polymer) to provide a base of the electrophoretic sample cell outer chamber, wherein said porous material allows current to flow between the outer and inner chambers of the electrophoretic cell, but minimizes and/or eliminates flow of analyte between the outer and inner chambers of the electrophoretic cell. In some embodiments, a composition comprising monomers is provided and a user polymerizes said monomers to provide said base in constructing the electrophoretic sample cell for use. In some embodiments, the porous matrix of the base is additionally blocked by deposition (e.g., electrophoretic deposition) of a charged, branched polymer (e.g., dextran sulfate) that is introduced into the inner chamber and migrated into the base by application of an electrical voltage prior to addition of the analyte to the sample chamber.

In some embodiments, the substrate comprises an immobilized capture probe. In some embodiments, the query probe is a fluorescently labeled nucleic acid that associates repeatedly with the analyte with a kinetic rate constant k_{off} that is greater than 0.1 min⁻¹ and/or a kinetic rate constant kₒₙ that is greater than 0.1 min⁻¹. In some embodiments, the system configured to detect said analyte comprises a fluorescence detector. In some embodiments, the system is configured to detect said analyte comprises a software component configured to fit a two-state model to query probe binding data. In some embodiments, the system further comprises a source of a voltage (e.g., a direct current or alternating current source). In some embodiments, the inner chamber of the electrophoretic cell comprises a restricted opening having an area of 0.01 to 10 mm (e.g., 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, or 10 mm). These various components of systems (e.g., electrophoretic cells, computers, inner and outer chambers, query probes, capture probes, substrates, and aspects of kinetic detection) are more fully described throughout the application, the relevant portions of which are incorporated here by reference.

Embodiments of the technology relate to compositions, e.g., an aqueous two-phase system (ATPS). In some embodiments, the ATPS comprises polyethylene glycol (PEG) and citrate. In some embodiments, the ATPS further comprises a salt (e.g., sodium chloride). In some embodiments, of the ATPS, the ATPS comprises a mass fraction of PEG greater than 30%, a mass fraction of citrate less than 2.5%, and a concentration of the salt is greater than 0.25 M. In some embodiments, the molecular weight of said PEG is between 1000 and 6000 Da. In some embodiments, said citrate is present as citric acid and sodium citrate in a ratio to provide a pH between 5.5 and 8.5.

That is, in some embodiments, the technology provides a composition (e.g., an ATPS) comprising PEG having a mass fraction in the composition that is greater than 30% (e.g., greater than 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 70, 71, 72, 73, 74, or 75%; comprising citrate having a mass fraction in the composition of less than 2.5% (e.g., less than 1.00, 1.25, 1.50, 1.75, 2.00, 2.25, or 2.50%). In some embodiments, the technology provides a composition comprising sodium chloride at a concentration of more than 0.25 M (e.g., more than 0.25, 0.3, 0.35, 0.4, 0.45, 0.5, 0.55, 0.6, 0.65, 0.7, 0.75, 0.8, 0.85, 0.9, 1.0, 1.1, 1.2, 1.3, 1.4, or more than 1.5 M). In some embodiments, the PEG has mean molecular weight in the composition of between 600 and 8000 Da (e.g., 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 7000, or 8000 Da). In some embodiments, the ratio of citric acid to sodium citrate in the composition provides a pH between 5.5 and 8.5 (e.g., a pH of 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5).

In some embodiments, the ATPS further comprises an analyte. In some embodiments, the ATPS comprises a first phase and a second phase and the first phase comprises a concentration of said analyte that is more than the concentration of the analyte in the second phase (e.g., at least approximately 2, 3, 4, 5, 6, 7, 8, 9, 10, 11, 12, 13, 14, 15, 16, 17, 18, 19, or 20 times the concentration of the analyte in the second phase). In some embodiments, the ATPS comprises a first phase and a second phase and the second phase comprises a volume that is more than the volume of the first phase (e.g., at least approximately 2, 3, 4, 5, 6, 7, 8, 9, 10, 11, 12, 13, 14, 15, 16, 17, 18, 19, or 20 times the volume of the first phase).

In some embodiments, the analyte is a nucleic acid (e.g., a nucleic acid comprising fewer than 200 basepairs or nucleotides (e.g., fewer than 200, 195, 190, 185, 180, 185, 180, 175, 170, 165, 160, 155, 150, 145, 140, 135, 130, 125, 120, 115, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 basepairs or nucleotides). In some embodiments, the analyte is a nucleic acid comprising fewer than 40 basepairs or nucleotides (e.g., fewer than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 basepairs or nucleotides). In some embodiments, the analyte is a single-stranded nucleic acid. In some embodiments, the analyte is a double-stranded nucleic acid. Further descriptions of capture probes, query probes, and analytes are provided elsewhere herein.

Some embodiments are related to kits. For example, embodiments provide a kit comprising polyethylene glycol (PEG) and citrate. In some embodiments, kits further comprise a sample well comprising a restricted opening. Some embodiments comprise an electrophoretic sample cell, a voltage source, and/or a porous material (e.g., a polymer) to provide a porous base of the outer chamber of the sample cell as described herein. Some kits comprise a substrate for capture and/or immobilization of an analyte. ATPS sample wells and electrophoretic sample cells are described elsewhere herein and the appropriate descriptions are incorporated herein by reference.

In some embodiments, the methods, systems, ATPS compositions, and kits find use in the analysis of a single cell, organelle, membraneless organelle, cell cluster, vesicle, (e.g., exosome), or molecule.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A-FIG. 1C show use of an aqueous two-phase system to concentrate a DNA biomarker near a surface for increased capture efficiency and assay sensitivity, as demonstrated by increasing the detected molecules in a SiMREPS assay for double-stranded mutant DNA. FIG. 1A is a drawing showing the steps of an embodiment of the technology using an aqueous two-phase system to concentrate a biomarker near a surface for increased capture efficiency and assay sensitivity. In FIG. 1a, a DNA analyte is concentrated in the minority phase (e.g., the phase having a smaller volume) of an aqueous two-phase system and the analyte is subsequently captured from the minority phase at a surface for analysis (e.g., by single-molecule analysis (e.g., by SiMREPS as described herein)). Centrifugation is typically carried out at approximately 1000×g for 10-120 minutes to provide high capture efficiency of the analyte at the coverslip surface. However, experiments conducted during the development of the technology provided herein indicated that centrifuging for approximately 1 minute at 1000×g is sufficient to provide phase separation.

FIG. 1B shows two photographs of analytes captured and imaged on a surface without concentration according to the ATPS technology provided herein (“−ATPS”) and after concentration according to the APTS technology provided herein (“+ATPS”). Each photograph shows a microscope field of approximately 52×52 μm². In FIG. 1b, the analyte is a 28-base-pair fragment of DNA encoding a T790M substitution of EGFR, a clinically important EGFR mutation causing therapeutic resistance in non-small cell lung cancer.

FIG. 1C is a plot showing that the sensitivity of kinetic fingerprinting for the EGFR variant T790M increases by a factor of greater than 80 using an aqueous two-phase system (e.g., comprising PEG 3350 (e.g., approximately 39% w/w), sodium citrate (e.g., approximately 1.2 w/w), and sodium chloride (e.g., approximately 2.8% w/w)). Surface-captured EGFR variant T790M analytes were imaged and counted by image analysis. A density of 25 analytes immobilized on the surface per approximately 118×118 μm² area were counted in the absence of concentration according to the ATPS technology provided herein. In contrast, a density of 2001 analytes immobilized on the surface per the same approximately 118×118 μm² area were counted after concentrating the analytes at the surface according to the ATPS technology provided herein.

FIG. 2 shows a drawing and photograph of custom sample wells creating a restricted contact area (0.01-1 mm²) with the underlying substrate to permit focusing of the analyte-rich phase over a small region of the solid support (e.g., coverslip) for efficient surface capture and detection of the analyte.

FIG. 3A-FIG. 3E shows detection of analytes with high specificity and single-molecule sensitivity using single-molecule recognition through equilibrium Poisson sampling (SiMREPS), or single-molecule kinetic fingerprinting. FIG. 3A is a diagram depicting the experimental SiMREPS approach adapted for DNA detection. Double-stranded (duplex) DNA is converted to single-stranded DNA with brief heat-denaturation in the presence of high concentrations of single-stranded dT₁₀ carrier to disfavor re-annealing of complementary analyte strands. Single-stranded target DNA is captured by target gene-specific locked nucleic acid capture strands immobilized on the slide surface, and remaining unbound DNA is washed away prior to kinetic fingerprinting with a mutant-specific fluorescent probe that is optimized to have fast binding and dissociation kinetics. FIG. 3B shows the effect of DNA strandedness and brief heat denaturation on the number of detected molecules (shown as “counts”) for two mutant target DNA alleles. Data are presented as means±s.e.m. of n=3 independent measurements. FIG. 3C through FIG. 3D show representative kinetic traces using a mutant-specific fluorescent probe with mutant DNA (MUT DNA), with wild-type DNA (WT DNA), or without DNA (No DNA control) for FIG. 3C EGFR Exon 19/deletion (c.2236_2250 del15) and FIG. 3D EGFR Exon 20 T790/M (c.2369C>T). The fluorescence intensity traces recorded during the experiments are shown in black (“noisy” trace); the 2-state idealization from hidden Markov modeling is shown as a grey line (smooth, squared fit of the “noisy” trace). FIG. 3E Standard curves from SiMREPS assays for EGFR Exon 19 deletion and EGFR Exon 20 T790M. Error-weighted linear fits were constrained to a y-intercept of 0; R²=0.982 (Exon 19 deletion), 0.999 (T790M). Data are presented as mean±s.e.m. of n=3 independent measurements. FOV, field of view.

FIG. 4 is a binodal curve plotted from data collected from a cloud-point turbidity assay. The data indicate the concentrations of sodium citrate dihydrate and PEG-3350 that produce a single aqueous phase (below the curve) or two aqueous phases (above the curve) in the presence of 2.8% w/w NaCl.

FIG. 5 is a plot showing the number of molecules (“Accepted Counts”) of a 28-nucleotide DNA oligonucleotide detected by SiMREPS kinetic fingerprinting per field of view after 1-hour incubation in three different matrices; PBS (100 μL sample volume), PBS+PEG/sodium citrate/NaCl ATPS (100 μL sample volume in PBS prior to addition to ATPS-forming components); PBS+PEG/sodium citrate/NaCl ATPS (20 μL sample volume in PBS prior to addition to ATPS-forming components).

FIG. 6 is a plot showing PCR-free detection of endogenous low-molecular weight DNA fragments from the EGFR gene, isolated from a human urine sample, and then analyzed by SiMREPS kinetic fingerprinting with the aid of an aqueous two-phase system composed of PEG 3350 (39% w/w), sodium citrate (1.2% w/w), and NaCl (2.8% w/w). The aqueous two-phase system yields sufficient sensitivity to detect wild-type DNA with the wild-type probe (WT), but mutant DNA is not detected at levels above background (MUT probe).

FIG. 7 is a plot showing the increase in assay sensitivity provided by including a complementary oligonucleotide to prevent re-hybridization of double-stranded DNA following thermal denaturation and concentration in an aqueous two-phase system. A 28-base-pair double-stranded DNA fragment (10 fM of a target DNA corresponding to a region of the EGFR gene) was heat-denatured in the presence (+Tile) or absence (−Tile) of a 14-base oligonucleotide (“Tile”) that is partly complementary to the target strand of the 28-base-pair duplex, but which does not obscure the remaining 14 base pairs (which would interfere with surface capture). The mixture is cooled following heat denaturation, then mixed thoroughly with lyophilized components to form an aqueous two-phase system (1.2% w/w sodium citrate, 2.8% w/w NaCl, 39% w/w PEG 3350) to concentrate the DNA for immobilization on a coverslip coated with an LNA-containing capture probe that is partly complementary to the target strand of the 28-base-pair duplex.

FIG. 8 is a plot showing that longer double-stranded DNA targets are captured with higher efficiency when complementary short oligonucleotides (e.g., 14 base pairs in length), or “tiles,” are included to prevent re-hybridization of the two complementary strands of the target. Data were collected from experiments in which tiles were used to improve capture of a 60 bp and 160 bp fragment of the EGFR gene containing the T790M mutation. Capture efficiency is increased ˜8- to 9-fold by inclusion of tile oligos.

FIG. 9 is a schematic drawing of an embodiment of an electrophoretic sample cell as described herein.

FIG. 10 is a photograph of an embodiment of an electrophoretic sample cell constructed and tested during the development of embodiments of the technology described herein.

FIG. 11A is a schematic drawing describing use of an electrophoretic sample cell to concentrate an analyte (e.g., DNA) at a surface (e.g., a coverslip).

FIG. 11B is a plot showing a voltage applied to an embodiment of an electrophoretic sample cell as described herein and the normalized DNA concentration at a surface as a function of time. The voltage cycled between 0 V and approximately 170 V; the DNA concentration at the surface was detected by measuring the fluorescence at the surface of a fluorescent label attached to the DNA. The DNA concentration varied between approximately 0-50 to approximately 350 (unitless, normalized concentration) and is shown by the trace with the high peaks at around 10-15 minutes. The smoothed line at approximately a concentration of 50-100 is the running average of the normalized DNA concentration at the surface.

FIG. 12A shows an image of a polyacrylamide electrophoresis gel experiment quantifying the partitioning of a 28-nucleotide single-stranded DNA in a PEG/sodium citrate/sodium chloride aqueous two-phase system.

FIG. 12B is a bar plot showing the quantification of gel bands from FIG. 12A to measure the amount of the 28-nucleotide single-stranded DNA in the PEG and sodium citrate/sodium chloride phases after equilibration. The citrate-rich phase was diluted 500-fold relative to the PEG-rich phase prior to loading on the gel to permit simultaneous quantification of DNA in the two lanes on a single instrument without saturating the detector. Staining was performed with SYBR gold.

FIG. 13 is a bar plot showing the quantification of partitioning of a 28-nucleotide single-stranded DNA oligonucleotide in a PEG/sodium citrate ATPS in the presence or absence of 2.8% w/w NaCl as measured by polyacrylamide gel electrophoresis followed by SYBR gold staining. The citrate-rich phase was diluted 500-fold relative to the PEG-rich phase prior to loading on the gel to permit simultaneous quantification of DNA in the two lanes on a single instrument without saturating the detector. Error bars represent +/−one standard deviation based on three independent replicates.

FIG. 14 is a bar plot showing the quantification of a microRNA by SiMREPS with and without pre-enrichment with a PEG/sodium citrate/NaCl ATPS. The apparent increase in sensitivity with the use of this ATPS is approximately 9-fold. Error bars represent one standard deviation from two independent replicates.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detection of analytes and particularly, but not exclusively, to compositions, methods, and systems for concentrating an analyte at a surface, e.g., for imaging and detection of low-abundance analytes.

Detection of analytes (e.g., biomolecules) at very low concentrations (e.g., less than 10⁻¹⁵ M or 1 femtomolar, fM) is of great interest in medical diagnostics (e.g., for non-invasive liquid biopsy), research (e.g., for single-cell analysis), and more broadly in analytical chemistry, but is challenging to achieve for most analytes. For instance, conventional enzyme-linked immunosorbent assays (ELISA) can typically achieve limits of detection in the range of 10-1000 fM (see, e.g., Rissin et al. (2010) “Single-molecule enzyme-linked immunosorbent assay detects serum proteins at subfemtomolar concentrations” Nat. Biotechnol. 28: 595-99). While nucleic acids can be detected at absolute quantities of <10 copies with amplification (e.g., by methods exploiting the polymerase chain reaction, PCR), such amplification introduces artifacts (see, e.g., Potapov & Ong (2017) 0 “Examining Sources of Error in PCR by Single-Molecule Sequencing” PLOS ONE 12: e0169774) such as copying errors, thermally induced chemical damage such as cytosine deamination to uracil, and sequence-dependent bias in the amplification or in the ligation of adapter sequences required for some protocols (see, e.g., Thiel et al. (2011) “Nucleotide Bias Observed with a Short SELEX RNA Aptamer Library” Nucleic Acid Ther. 21: 253-63; Paweletz et al. (2016) “Bias-Corrected Targeted Next-Generation Sequencing for Rapid, Multiplexed Detection of Actionable Alterations in Cell-Free DNA from Advanced Lung Cancer Patients” Clin. Cancer Res. 22: 915-22). Furthermore, amplification of DNA or RNA typically does not copy epigenetic modifications such as modified bases (e.g., 5-methylcytosine, 5-hydroxymethylcytosine, or N⁶-methyladenine), necessitating bisulfite treatment to distinguish between modified and unmodified bases; however, bisulfite treatment leads to fragmentation and damage of up to 80% of the DNA (see, e.g., Grunau et al. (2001) “Bisulfite genomic sequencing: systematic investigation of critical experimental parameters” Nucleic Acids Res. 29: e65), and cannot distinguish between many types of biologically important modifications (such as distinguishing between 5-methylcytosine and 5-hydroxymethylcytosine; see, e.g., Huang et al. (2010) “The Behaviour of 5-Hydroxymethylcytosine in Bisulfite Sequencing” PLOS ONE 5: e8888). Finally, short fragments of nucleic acids (<40 base pairs), such as those found in trans-renal DNA of urine, are challenging or impossible to amplify directly by PCR due to their small size and the length requirements of PCR primers. Analysis of many analytes isolated from single cells is also inefficient, even with PCR-based methods (in the case of nucleic acids), due to the extremely low overall input material, low copy numbers of many analytes, sequence bias of ligases, and low efficiency of many processing enzymes at such low inputs. Some ultra-sensitive next-generation methods have reported low-femtomolar and sub-femtomolar limits of detection, but typically still possess detection limits several orders of magnitude above the level of single copies of the analyte (see, e.g., Rissin et al. (2010) “Single-molecule enzyme-linked immunosorbent assay detects serum proteins at subfemtomolar concentrations” Nat. Biotechnol. 28: 595-99 (2010); Cohen et al. (2017) “Digital direct detection of microRNAs using single molecule arrays” Nucleic Acids Res. 45: e137).

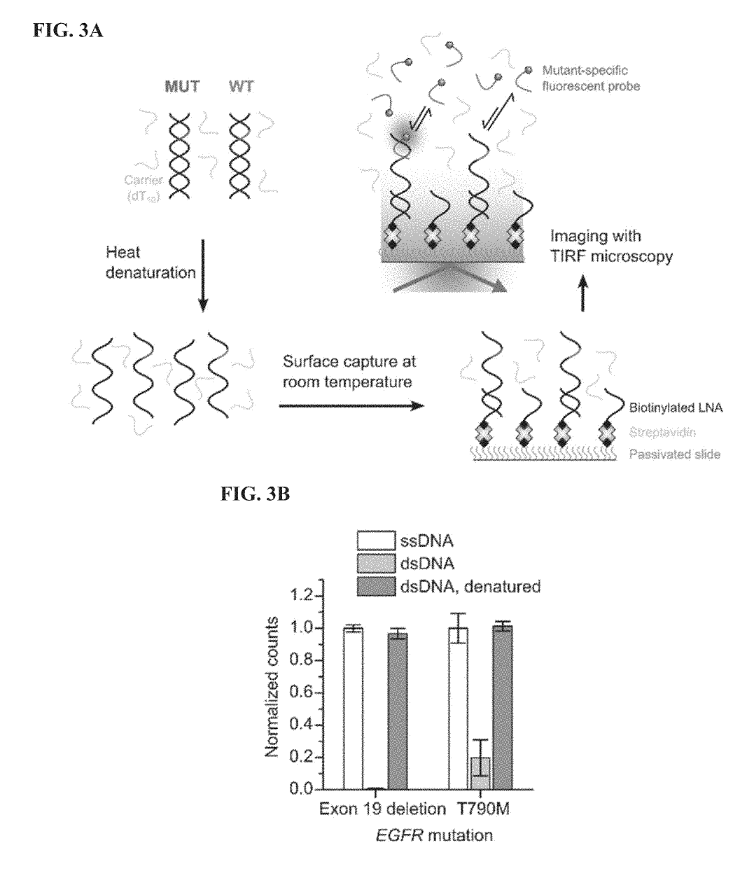

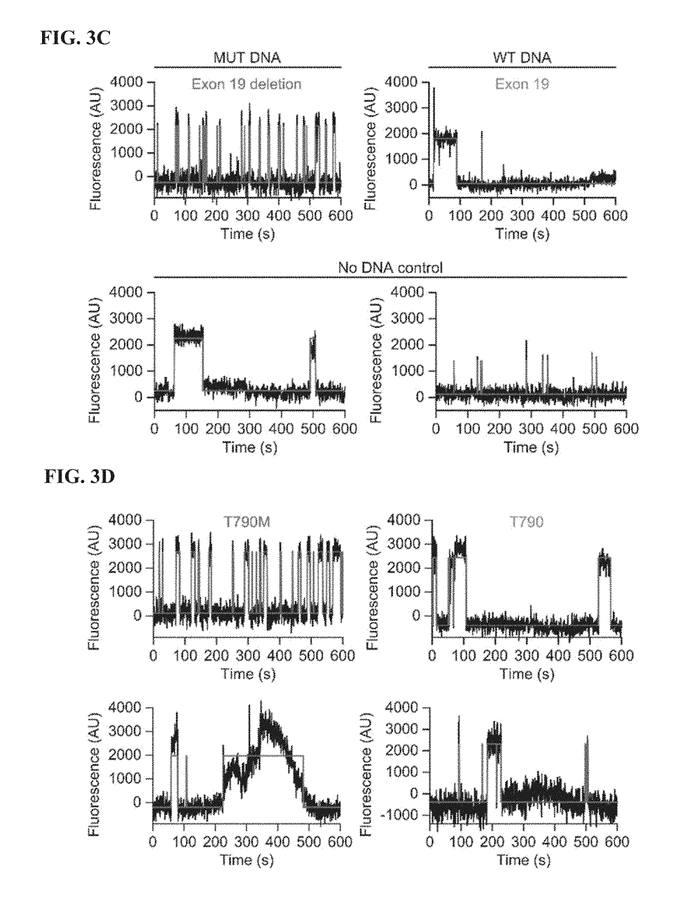

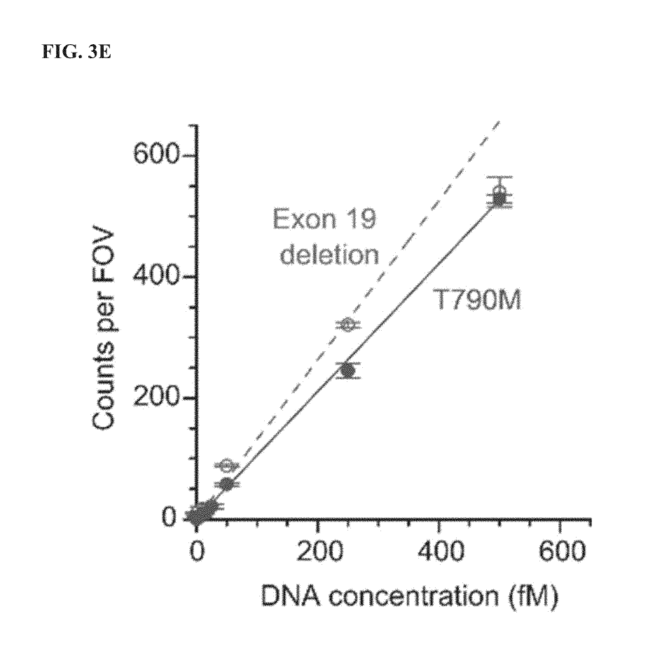

Co-pending applications U.S. patent application Ser. No. 14/589,467; Int'l Pat. App. No. PCT/US2015/044650; Int'l Pat. App. No. PCT/US2017/016977; and U.S. Provisional App. Ser. No. 62/468,578 (each of which is incorporated herein by reference in its entirety) describe a technology called “single-molecule recognition through equilibrium Poisson sampling” (SiMREPS) that detects the presence of single analytes (e.g., microRNA molecules) with theoretically unlimited precision (see also, e.g., Johnson-Buck et al. (2015) “Kinetic fingerprinting to identify and count single nucleic acids” Nat. Biotechnol. 33: 730-32). In this approach, an analyte of interest is captured in a discrete region of a solid support, generally using a stably binding capture probe (such as a locked nucleic acid probe), and then a second epitope or binding region of the analyte is probed repeatedly by a query probe. In some embodiments, this technology detects analytes such as single-stranded nucleic acids; further, in some embodiments, this technology also detects double-stranded nucleic acids by employing a denaturation protocol prior to surface capture (see, e.g., FIG. 3a), and exhibits a specificity for a point mutation (EGFR T790M) greater than 99.99998%. However, while the approach detects single molecules, data has indicated that a limiting factor in achieving a very low limit of detection (sub-femtomolar, and ultimately comparable to PCR-based methods) is in achieving efficient mass transport of the analyte to the solid support for detection.

Accordingly, the technology described herein relates to increasing the efficiency of mass transport of an analyte to a solid support (e.g., increasing the fraction or amount of analyte captured on a region of the solid support and/or capable of being measured in a reasonable amount of measurement time). During the development of embodiments of the technology provided herein, experiments were conducted to increase mass transport of an analyte to a solid support by a number of strategies, including but not limited to: (1) increasing the rate constant of association between the analyte and the capture probe or the solid support, such as by modifying the pH, salt composition or concentration; (2) increasing the effective or actual concentration of the analyte in the proximity of the solid support, such as by evaporation, application of an electric field (for charged analytes), or introduction of molecular crowding agents such as polyethylene glycol (PEG) or dextran; and/or (3) increasing the incubation time (see, e.g., Example 10).

Experiments were conducted in which these strategies were tested to provide immobilized analytes for SiMREPS experiments and data were collected describing the effects of these methods on the sensitivity of SiMREPS assays, in particular for detecting nucleic acids. The data indicated approximately 2- to 5-fold improvements in the sensitivity of the assay for 28-nucleotide fragments of DNA containing an EGFR mutation (see, e.g., Example 10). In these assays, the relative sensitivity was determined by determining the positive detection events per field of view (FOV) after employing the indicated strategy, dividing this by the positive detection events per field FOV using the standard incubation conditions (PBS buffer, 1 hour), and rounding to the nearest integer. Surprisingly, increasing the capture incubation time 18-fold yielded only an approximately 2-fold increase in sensitivity, indicating that the capture reaction reaches equilibrium in much less than 18 hours, and that either the finite affinity of the capture probe or nonspecific analyte adsorption to the sample cell walls limit the amount of analyte that can be captured. Incubation in different buffers, with additives often used in hybridization reactions (e.g., formamide, Tween-20, dextran sulfate, etc.) yielded similarly poor improvements in sensitivity. Decreasing the capture area (to <1 mm² from a standard area of 25 mm²) was expected to increase the density of captured analyte by a factor of up to 25, but instead yielded an approximately 3-fold improvement. Repeated mixing of the solution was also attempted using a syringe pump to overcome limitations of capture efficiency due to slow diffusion of DNA over distances >1 mm; however, this yielded no significant improvement in sensitivity. Perhaps most surprisingly, no significant improvement was seen with ethanol precipitation followed by resuspension of the analyte in a smaller volume of solution, perhaps because the process of ethanol precipitation damaged the capture surface (e.g., by microscale abrasion of the surface by the precipitate crystals, or the introduction of an organic solvent). While ethanol precipitation and resuspension in a smaller volume can in principle be performed in a separate vessel, this introduces practical challenges in terms of efficiently resuspending small amounts of material in a very small volume (e.g. 1 μL) and efficiently transferring this small volume. Evaporation of 50-99% of the solvent after dispensing the sample into the sample well fared better, but yielded no more than a 5-fold increase in sensitivity, even with far greater than a 5-fold reduction in sample volume (e.g. near-total evaporation, leaving <5% of the sample volume), even when buffer components were reduced in concentration up to 10-fold to offset problems of buffer and salt crystallization upon solvent evaporation.

As indicated by experiments conducted during the development of embodiments of the technology described herein, successful approaches to improve the sensitivity of a SiMREPS assay include, e.g., combining SiMREPS with pre-concentration of the analyte using an aqueous two-phase system (ATPS) or an electrophoretic sample cell.

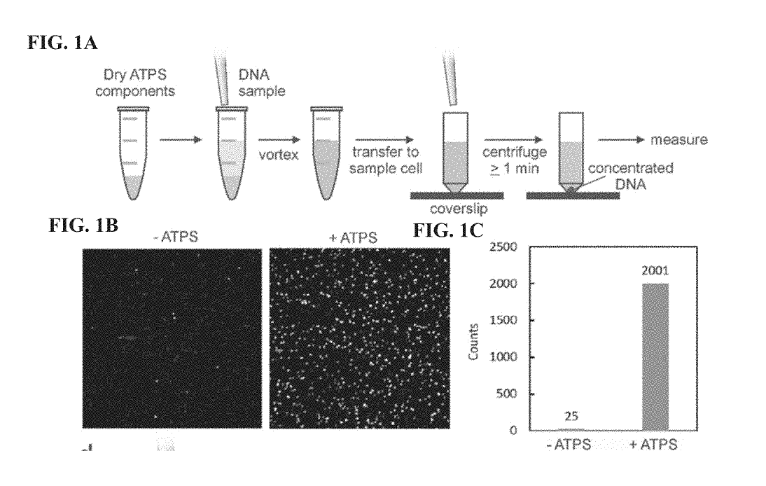

An aqueous two-phase system, or ATPS (Iqbal et al. (2016) “Aqueous two-phase system (ATPS): an overview and advances in its applications” Biol. Proced. Online 18), arises when two water-soluble components—e.g., two polymers or a polymer and a salt—exceed a threshold concentration and separate into two aqueous phases with distinct compositions. Several types of ATPS can selectively partition biomolecules, including DNA and proteins, into one of the two phases, sometimes with a >1000-fold preference for one of the phases (Alberts (1967) “Efficient Separation of Single-Stranded and Double-Stranded Deoxyribonucleic Acid in a Dextran-Polyethylene Glycol Two-Phase System” Biochemistry (Mosc.) 6: 2527-32). When one of the two ATPS components is present in excess relative to the other, the two resulting phases can have unequal volumes at equilibrium, thus concentrating a biomolecule of interest in the smaller phase (Iqbal et al. (2016) “Aqueous two-phase system (ATPS): an overview and advances in its applications” Biol. Proced. Online 18). As one specific embodiment of this approach to enhancing sensitivity of a single-molecule assay, the technology comprises use of ATPS to concentrate very dilute (approximately equal to or less than 10 fM) solutions of DNA over a small region of the imaging surface for rapid and efficient capture of the analyte (see, e.g., Example 1 and FIG. 1). In this approach, a DNA sample is heat-denatured, mixed vigorously with ATPS-forming components, then briefly centrifuged to position the DNA-rich lower phase near the imaging surface. The method is a straightforward process that is completed in only a few minutes, though often the centrifugation is continued for 10, 30, 60, or 120 minutes to increase the amount of contact time between the analyte-rich phase and the solid support onto which it is being captured (such as a coverslip). Alternatively, a brief centrifugation (e.g., for 1 minute) may be employed to separate the phases of the ATPS, followed by a longer incubation period of 10, 30, 60, or 120 minutes (or up to 24 hours) without centrifugation for analyte capture.

Several ATPS compositions have been shown to selectively partition DNA, including polyethylene glycol (PEG)/sodium citrate (SC) (see, e.g., Gomes et al. (2009) “Purification of plasmid DNA with aqueous two phase systems of PEG 600 and sodium citrate/ammonium sulfate” Sep. Purif. Technol. 65: 22-30), PEG/sodium sulfate (see, e.g., Nazer et al. (2017) “Partitioning of pyrimidine single stranded oligonucleotide using polyethylene glycol-sodium sulfate aqueous two-phase systems; experimental and modeling” Fluid Phase Equilibria 432: 45-53), and PEG/dextran (see, e.g., Alberts (1967) “Efficient Separation of Single-Stranded and Double-Stranded Deoxyribonucleic Acid in a Dextran-Polyethylene Glycol Two-Phase System” Biochemistry (Mosc.) 6: 2527-32). While plasmid DNA has been shown to exhibit as much as 100- to 1000-fold selectivity for one phase over the other (see, e.g., Alberts, supra; see e.g., Nazer, supra), and one study has examined the partitioning of a single-stranded polypyrimidine DNA molecule about 20 nucleotides in length in a PEG/sodium sulfate system (see, e.g., Nazer, supra), it was previously not predictable or known if ATPS technologies concentrate short nucleic acid fragments (e.g., double-stranded DNA fragments found in biofluids, short RNAs such as miRNAs, or single-stranded DNAs containing both purine and pyrimidine bases of varying sequence) into small volumes. In addition, the practical utility of PEG/sodium sulfate ATPS is limited by the poor solubility of sodium sulfate in water, often resulting in precipitation on the formation of ATPS, which limits the applicability for surface-based assays in particular.

Further, while a strategy for capturing sub-nanogram inputs of DNA for sequencing using electrophoresis has recently been reported (Larkin et al. (2017) “Length-independent DNA packing into nanopore zero-mode waveguides for low-input DNA sequencing” Nat. Nanotechnol.), this technology requires electron-beam lithography to generate an array of nanopores in a membrane placed below the sensor chip (Larkin et al. (2014) “Reversible Positioning of Single Molecules inside Zero-Mode Waveguides” Nano Lett. 14: 6023-29), which adds to the already nontrivial cost of fabricating zero-mode waveguides used in that sequencing platform. In contrast, the electrophoretic concentration methods described herein provide a low-cost and general approach for efficient surface-based analysis of samples comprising dilute analytes of many types.

Based on data collected during the development of SiMREPS and other experiments detecting analytes, criteria were established for ATPS and electrophoretic concentration methods appropriate for use with SiMREPS and other sensitive analyte detection technologies. The primary purpose of ATPS and electrophoretic concentration methods is efficiently partitioning an analyte of interest into a smaller volume, yielding a net increase in the concentration of the analyte. In addition, the technology: (1) is non-destructive of the surface chemistry required for analyte capture (e.g., streptavidin-biotin interaction); (2) is non-interfering with passivation of the solid support (e.g., by a coating of PEG, PEG-biotin, or hydrophobic molecules), which could increase nonspecific binding of the query probe to prohibitive levels during a single-molecule assay, resulting in false positive or false negative signal; and (3) is non-interfering with the capture chemistry (e.g., not decreasing the affinity of the analyte for the solid support or capture probe, and not decreasing the kinetics of binding of the analyte to the solid support).

Further, an appropriate ATPS technology preferably partitions the analyte into the denser of the two phases, permitting contact between the analyte-rich phase and the solid support by gravity or centrifugation (see, e.g., FIG. 1a), although enrichment in the less dense phase could also be utilized by inverting the relative orientations of the sample well and the solid support, as long as accumulation of air bubbles is prevented.

Prior to experiments conducted during the development of embodiments of the technology provided herein, it was not known or predictable that an aqueous two-phase system would satisfy all of the above requirements for a given analyte and SiMREPS assay; indeed, while the data collected during these experiments indicated that a PEG/dextran ATPS partitions short single-stranded DNA quite efficiently, PEG/dextran ATPS methods were not as successful at improving delivery of analyte to a substrate and/or improving sensitivity of SiMREPS as an ATPS based on PEG/sodium citrate (see, e.g., Example 10). Without being bound by theory, the limitations of the PEG/dextran ATPS may result from the high viscosity of the dextran-rich and DNA-rich phase, which may limit capture kinetics.

For the application of ATPS to short single-stranded and double-stranded DNA (e.g., less than 200, 190, 180, 170, 160, 150, 140, 130, 120, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, or fewer than 10 nt or bp), data collected during the development of embodiments of the technology described herein indicated that an ATPS comprising a mixture of PEG 3350, sodium citrate, and sodium chloride effectively partitions and concentrates short single-stranded or double-stranded DNA (e.g., as small as 20-30 bp in length), increasing the sensitivity of the SiMREPS assay for EGFR T790M by a factor of approximately 80 (FIG. 1b; FIG. 1c), which indicates detection of approximately 100 molecular copies and thus approaching the sensitivity of PCR-based analysis. Spectrophotometry data collected during the development of embodiments of the technology described herein further indicated the ability of an ATPS comprising a mixture of PEG 3350, sodium citrate, and sodium chloride to partition DNA as small as 8 nucleotides in length preferentially into the citrate-rich phase, albeit with a lower partition coefficient than for 20-nucleotide DNA strands, and in a manner that is easily perturbed by chemical modifications of the DNA (for instance, conjugation of the organic fluorophore Cy5 to an 8-nucleotide DNA single-stranded DNA molecule caused the DNA to partition preferentially into the PEG-rich phase, rather than the citrate-rich phase; such an inversion was not observed for DNA molecules longer than 20 nucleotides).

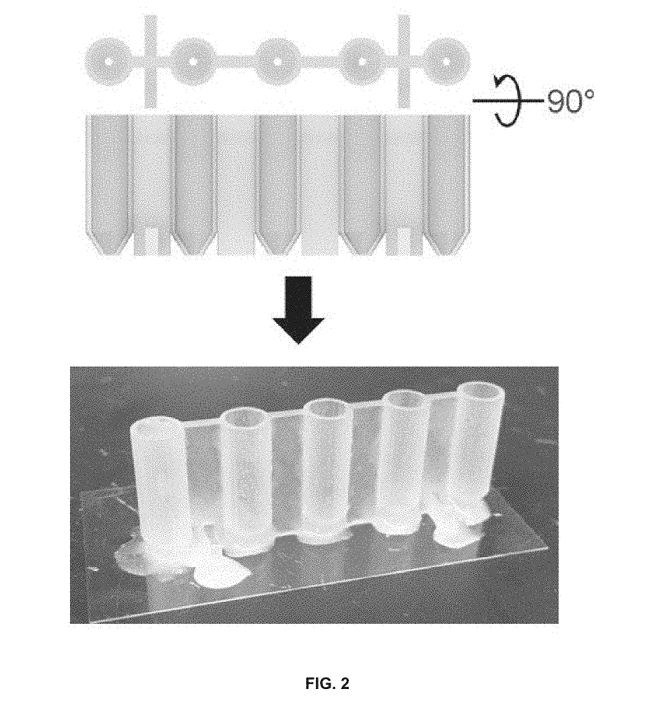

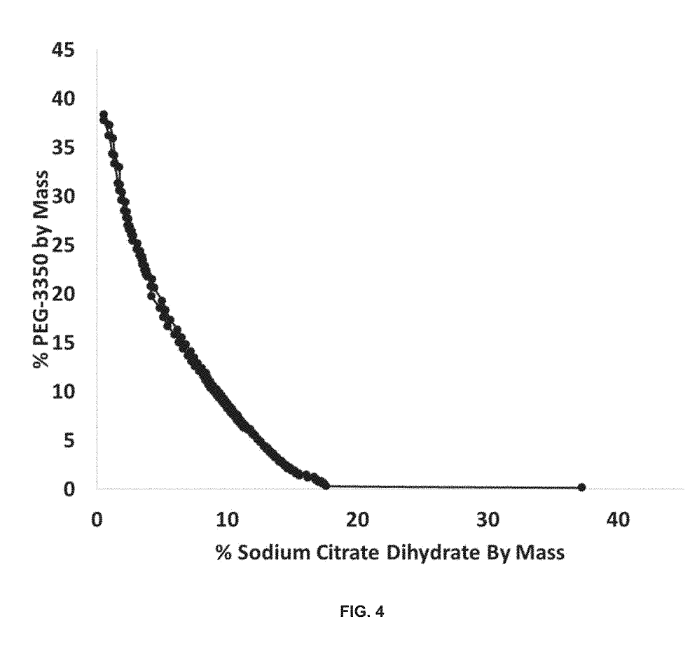

In some embodiments, the sensitivity of this assay is improved by the use of custom sample cells that make contact with the solid support over a narrow, well-defined area for analyte capture (see, e.g., FIG. 2; Example 2). Such sample cells are optimally constructed to permit the access of a pipet or other mixing and dispensing implement while avoiding the potential trapping of solutions or air bubbles near the base (for example, by ensuring an angle of contact >45° (defined relative to the perpendicular) between the underlying solid support and the inner walls of the sample cell). When using embodiments of the ATPS system described herein, it is particularly important to maintain the overall concentrations of the two-phase-forming reagents (e.g., PEG and sodium citrate) above the thresholds required to form two aqueous phases. For instance, in the PEG-3350/sodium citrate/NaCl system, holding the concentration of NaCl constant, experiments were conducted using a cloud-point assay to identify mixture compositions producing one-phase or two-phase behavior (see, e.g., Example 4 and FIG. 4), separated by the so-called binodal line.

Furthermore, it is important to select a composition in which sodium citrate is much less abundant than PEG-3350 (e.g., 1-5% sodium citrate (e.g., 1, 1.5, 2, 2.5, 3, 3.5, 4, 4.5, or 5% sodium citrate) and 30-45% PEG-3350 (e.g., 30, 31, 32, 33, 34, 35, 36, 37, 38, 39, 40, 41, 42, 43, 44, or 45% PEG-3350) so that the DNA-rich citrate phase comprises a minority of the mass and volume of the system (e.g., <5%) and the DNA is thus concentrated by a factor of >10 relative to its original concentration. Data collected during the development of embodiments of the technology provided herein indicated that the addition of 1-15% (e.g., 1, 2, 3, 4, 5, 6, 7, 8, 9, 10, 11, 12, 13, 14, or 15%) sodium chloride enhances the partitioning of short nucleic acids, particularly in the range of 10-40 nucleotides, into the sodium citrate phase (see, e.g., Example 5). While NaCl has been previously reported in the literature to alter the partitioning of some proteins and change the phase behavior of PEG/sodium citrate mixtures (see, e.g., Azevedo et al. (2009) “Partitioning of human antibodies in polyethylene glycol-sodium citrate aqueous two-phase systems” Sep. Purif. Technol. 65: 14-21), it has not been previously known or predictable that NaCl addition enhances the partitioning of nucleic acids, and especially short oligonucleotides, in a PEG/citrate ATPS. In addition, data collected from experiments conducted during the development of embodiments of the technology described herein indicated a similar partitioning for fluorescently labeled and unlabeled DNA oligonucleotides, single-stranded and double-stranded DNA oligonucleotides, and both fluorescently labeled and unlabeled microRNAs (see, e.g., Example 9).

While ATPS are generally formed by mixing concentrated aqueous stocks of the phase-forming components, experiments conducted during the development of embodiments of the technology provided herein have indicated that it is important to omit as much water as possible from the stock mixtures to maximally concentrate dilute analytes for increased sensitivity in surface-based measurements. However, given the low mass fractions of some components required for a high concentration factor (e.g., a <1:10 ratio between citrate and PEG), and the small input volumes of many applications (e.g., <100 microliters), it is difficult to aliquot solid reagents such as PEG-3350, sodium citrate, and NaCl with a precision required for reproducible behavior (e.g., coefficient of variation <25% between repeat measurements) and sensitivity. Data collected during experiments conducted during the development of embodiments of the technology have indicated that this approach sometimes results in variable volumes of the two phases, and sometimes results in failure to form two phases. Pre-mixing larger quantities of the solid powders or neat liquids (e.g., in the case of PEG-600, which was sometimes substituted for PEG-3350 in the ATPS) yielded similarly poor reproducibility, likely because of the inherent granularity and discreteness of the solids. In contrast, experiments conducted during the development of embodiments of the technology provided herein indicated that preparing a single-phase master mix comprising the correct proportions of components (e.g., 6.934 grams of PEG-3350, 0.498 grams of NaCl, 0.208 grams of sodium citrate, and 30 mL deionized water), aliquoting and lyophilizing this mixture to achieve compositionally well-defined pellets of known mass, and then adding a set volume of analyte solution or mixture to these lyophilized pellets, yielded much more reproducible results (see, e.g., Example 5 and Table 1). The data collected during these experiments indicated that varying the sodium citrate concentration within this lyophilized mixture alters the fractional volume of the DNA-rich phase and, hence, alters the concentration of the DNA within the final mixture.

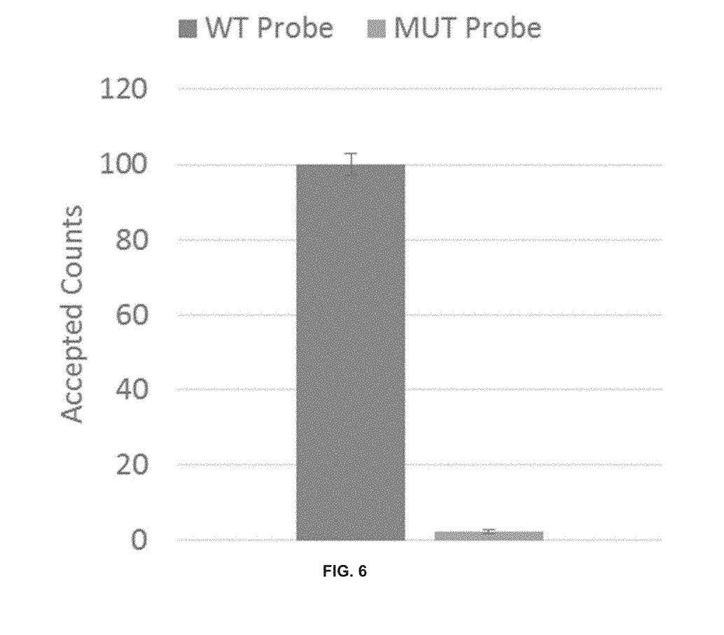

Experiments conducted during the development of embodiments of the technology described herein indicated several aspects relating to use of ATPS to increase the sensitivity of single-molecule and surface-based assays (e.g., SiMREPS assays for short double-stranded DNA). First, due to the high salt concentration, the PEG/sodium citrate/NaCl system performs robustly with a broad range of input compositions comprising analyte (e.g., deionized water, PBS, TE, TBE, or a biofluid (e.g., urine supernatant)) or an additive (e.g., SDS (e.g., 2%), proteinase K). Second, the pH of the sodium citrate/citric acid used in producing the lyophilized mixture may be varied between 6.5 and 8.5 with minimal impact on the sensitivity of the SiMREPS assay, though a slight decrease in counts is seen below a pH of about 7.0 (see, e.g., Example 11). Interestingly, while only one phase is seen at pH 5.91, a PEG/sodium citrate mixture still increases the sensitivity of the assay significantly relative to PBS alone, though not to the extent that an aqueous two-phase system of the same PEG/sodium citrate composition does. Third, the input volume of DNA solution may be decreased from 100 μL to 20 μL without a proportional drop in sensitivity, suggesting that a higher sensitivity (in terms of the fraction of total copies of the analyte detected) is provided with low input volumes that permit rapid diffusion of the analyte through the small volume of the lower phase to the solid support for capture. To demonstrate application to a clinically relevant specimen, using a PEG/sodium citrate/NaCl ATPS to enhance surface capture, experiments conducted during the development of the technology provided herein indicated that the technology detected significant positive signal (see, e.g., FIG. 6 and Example 7) for a fragment of the EGFR gene in DNA isolated from human urine and selected for low-molecular weight (<500 bp) DNA, a method intended to separate trans-renal DNA from DNA derived from the epithelium of the urinary tract. In contrast, no statistically significant positive signal for the T790M mutation (MUT) was detected in the healthy urine DNA sample, indicating that the ATPS does not interfere with specificity (see, e.g., FIG. 6 and Example 7). Furthermore, the low, statistically insignificant MUT signal was reduced further by treatment with uracil-DNA glycosylase to remove deaminated cytosine (uracil), which is a common source of false positives in biological assays for cytosine-to-thymine mutations such as T790M.

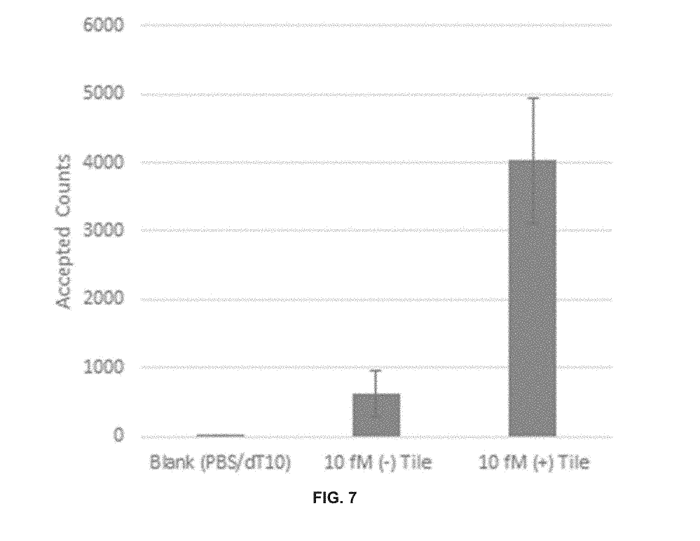

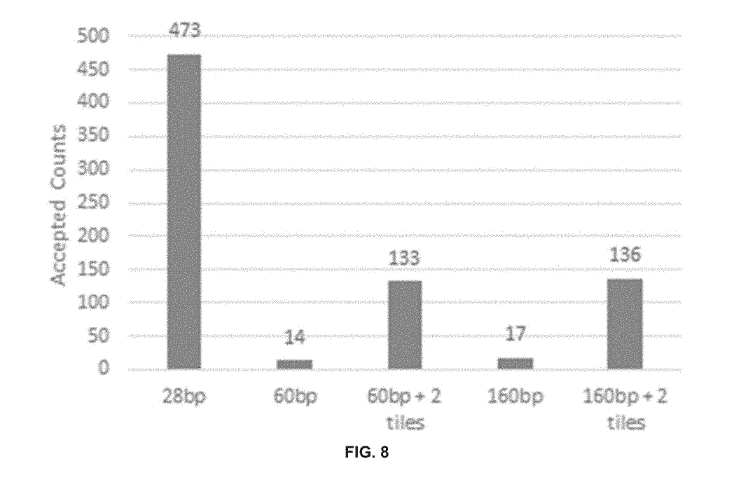

Another important point for the use of ATPS for double-stranded DNA, or other analytes that must be dissociated from binding partners before capture, is that the use of ATPS components (often molecular crowding agents or salts) may inadvertently promote re-binding or re-hybridization, which can reduce the efficiency of surface capture. For instance, high concentrations (e.g., >0.1 M) of salts like sodium citrate and NaCl can greatly increase the kinetics of DNA hybridization—often by several orders of magnitude (see, e.g., Dupuis et al. (2013) “Single-Molecule Kinetics Reveal Cation-Promoted DNA Duplex Formation Through Ordering of Single-Stranded Helices” Biophys. J 105: 756-66). Concentrating such materials into a smaller phase may have a similar, or even stronger, effect on re-binding, since association kinetics is almost always proportional to reagent concentration. The re-binding of the analyte to its binding partners can have a detrimental effect on sensitivity of an assay relying on its capture at a surface. To circumvent this problem, experiments were conducted during the development of the technology provided herein in which short complementary oligonucleotides were introduced at an excess relative to the analyte to prevent re-hybridization of the complementary strand once the DNA is concentrated in the citrate phase. The oligonucleotide was introduced prior to heat denaturation of the duplex DNA or immediately after denaturation. Data collected during these experiments indicated that the sensitivity of the SiMREPS assay for T790M increased by a factor of approximately 6 upon addition of this oligonucleotide (see, e.g., FIG. 7 and Example 8). Similarly, data collected during these experiments indicated that adding short complementary oligonucleotides to the sample promote the denaturation of longer (e.g., 60 or 160 base pair) DNA strands (see, e.g., FIG. 8 and Example 8) and/or to sequester secondary structures that may interfere with capture of the analyte at the surface or with probing by a query probe.

In summary, embodiments of the technology described herein relate to detecting very low copy numbers (e.g., approximately 100) of analyte without amplification by using an aqueous two-phase system to pre-concentrate the analyte and improve its capture efficiency at a surface, then detecting the analyte with a single-molecule-sensitive assay (e.g., SiMREPS). In some embodiments, ATPS are selected to promote efficient concentration of the analyte and to promote efficient capture. In some embodiments, the ATPS is compatible with downstream assay requirements such as surface chemistry and passivation against probe binding. While concentration can introduce additional sensitivity challenges, experiments conducted during the development of embodiments of the technology indicated that these can be overcome by the introduction of appropriate carriers or nonspecific inhibitors of rebinding (such as dT₁₀) or specific competitors (such as complementary tile oligonucleotides). The method finds use in clinical and biomedical applications, such as the detection of low-abundance and low-molecular-weight DNA fragments isolated from biofluids (e.g., urine). Its sensitivity and robustness to the addition of detergents and enzymes further makes it an attractive option for single-cell analysis, including multiplexed analysis of low-abundance components such as rare microRNAs, DNA sequences, and proteins.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Description

As described herein, the technology provides compositions, methods, and systems for moving and/or concentrating an analyte at a surface, e.g., for imaging and detection of low-abundance analytes. For example, in some embodiments, an analyte is concentrated at a surface using an electrophoretic cell (e.g., by using a voltage to move and concentrate an analyte at a surface). Furthermore, in other exemplary embodiments, an analyte (e.g., a nucleic acid (e.g., a DNA), a protein, etc.) is partitioned preferentially into one of two phases of an aqueous two-phase system. In some embodiments relating to aqueous two-phase systems, the volume of the analyte-rich phase is smaller than the original analyte mixture, which produces a higher concentration of analyte in the new analyte-rich phase.

For example, during the development of embodiments of the technology provided herein, experiments were conducted in which DNA was concentrated greater than 10-fold using an ATPS comprising PEG 3350, sodium citrate, and sodium chloride, and the concentrated DNA was subsequently captured on a surface for subsequent analysis. In some embodiments, the analyte-rich phase is exposed to (e.g., is contacted with) a surface for subsequent analysis (e.g., by methods such as (but not limited to) fluorescence microscopy, SiMREPS, etc.).

Furthermore, for example, during the development of embodiments of the technology provided herein, experiments were conducted in which DNA was concentrated at a surface greater than 10-fold using an electrophoretic cell. In some embodiments, a solution comprising an analyte is exposed to (e.g., is contacted with) a surface for subsequent analysis (e.g., by methods such as (but not limited to) fluorescence microscopy, SiMREPS, etc.).

In some embodiments, the surface is a coverslip, microscope slide, colloidal particle, or phase boundary. In some embodiments, the analyte is captured on the surface prior to analysis (e.g., using an affinity reagent). In some embodiments, the analyte is not captured on the surface prior to analysis.

In some embodiments, the technology finds use with a single-molecule detection assay (see, e.g., U.S. patent application Ser. No. 14/589,467; Int'l Pat. App. No. PCT/US2015/044650; Int'l Pat. App. No. PCT/US2017/016977; and U.S. Provisional App. Ser. No. 62/468,578; each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In some embodiments, the technology finds use in the qualitative detection (e.g., determining the presence) of an analyte. In some embodiments, the technology finds use in the quantitative measurement (e.g., enumeration) of the amount, concentration, abundance, etc. of an analyte. For example, in some embodiments the technology finds use to concentrate a low-abundance nucleic acid analyte present in a biofluid, e.g., tumor DNA from plasma, urine, saliva, or other body fluids into a smaller volume for analysis.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the terms “subject” and “patient” refer to any organisms including plants, microorganisms, and animals (e.g., mammals such as dogs, cats, livestock, and humans).

As used herein, the term “analyte” refers to a substance to be tested, assayed, detected, imaged, characterized, and/or quantified. Exemplary analytes include, but are not limited to, biomolecules (e.g., nucleic acids (e.g., DNA, RNA, methylated and other modified nucleobases), polypeptides (e.g., peptides, proteins, glycoproteins), carbohydrates, lipids, post-translational modifications, amino acids, and metabolites), small molecules (drugs, bioactive agents, toxins), etc.

The term “sample” in the present specification and claims is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

As used herein, the term “biofluid” refers to a biological fluid (e.g., a body fluid, a bodily fluid). For example, in some embodiments, a biofluids is an excretion (e.g., urine, sweat, exudate (e.g., including plant exudate)) and in some embodiments a biofluid is a secretion (e.g., breast milk, bile). In some embodiments, a biofluid is obtained using a needle (e.g., blood, cerebrospinal fluid, lymph). In some embodiments, a biofluid is produced as a result of a pathological process (e.g., a blister, cyst fluid). In some embodiments, a biofluid is derived from another biofluid (e.g., plasma, serum). Exemplary biofluids include, but are not limited to, amniotic fluid, aqueous humor, vitreous humor, bile, blood, blood plasma, blood serum, breast milk, cerebrospinal fluid, cerumen (earwax), chyle, chime, endolymph, perilymph, exudates, feces, female ejaculate, gastric acid, gastric juice, lymph, mucus (e.g., nasal drainage, phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit.

The term “label” as used herein refers to any atom, molecule, molecular complex (e.g., metal chelate), or colloidal particle (e.g., quantum dot, nanoparticle, microparticle, etc.) that can be used to provide a detectable (preferably quantifiable) effect, and that can be attached to a nucleic acid or protein. Labels include, but are not limited to, dyes (e.g., optically-detectable labels, fluorescent dyes or moieties, etc.); radiolabels such as ³²P; binding moieties such as biotin; haptens such as digoxgenin; luminogenic, phosphorescent, optically-detectable, or fluorogenic moieties; mass tags; and fluorescent dyes alone or in combination with moieties that can suppress or shift emission spectra by fluorescence resonance energy transfer (FRET). Labels may provide signals detectable by fluorescence, luminescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

“Support” or “solid support”, as used herein, refers to a surface or matrix on or in which an analyte (e.g., a biomolecule (e.g., nucleic acid (e.g., DNA, RNA, methylated and other modified nucleobases), polypeptide (e.g., peptide, protein, glycoprotein), carbohydrate, lipid, post-translational modification, amino acid, metabolite), small molecule, etc.) may be concentrated and/or immobilized, e.g., a surface to which an analyte may be covalently or noncovalently attached or in, or on which an analyte may be partially or completely embedded so that the analytes are largely or entirely prevented from diffusing freely or moving with respect to one another.

As used herein, “moiety” refers to one of two or more parts into which something may be divided, such as, for example, the various parts of an oligonucleotide, a molecule, a chemical group, a domain, a probe, etc.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the terms “complementary” or “complementarity” are used in reference to polynucleotides (e.g., a sequence of nucleotides such as an oligonucleotide capture probe, query probe or a target analyte that is a nucleic acid) related by the base-pairing rules. For example, for the sequence “5′-A-G-T-3′″ is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

“Mismatch” means a nucleobase of a first nucleic acid that is not capable of pairing with a nucleobase at a corresponding position of a second nucleic acid.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include transmembrane domains, glycosylation sites, etc.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; in some embodiments, one sequence is a reference sequence.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows—Alanine: Ala, A; Arginine: Arg, R; Asp aragine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

The terms “variant” and “mutant” when used in reference to a polypeptide refer to an amino acid sequence that differs by one or more amino acids from another, usually related polypeptide.

As used herein, the term “melting” when used in reference to a nucleic acid refers to the dissociation of a double-stranded nucleic acid or region of a nucleic acid into a single-stranded nucleic acid or region of a nucleic acid.

As used herein, a “query probe” or “reader probe” is any entity (e.g., molecule, biomolecule, etc.) that recognizes an analyte (e.g., binds to an analyte, e.g., binds specifically to an analyte). In exemplary embodiments, the query probe is a protein that recognizes an analyte. In some other exemplary embodiments, the query probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, the query probe is labeled, e.g., with a detectable label such as, e.g., a fluorescent moiety as described herein. In some embodiments, the query probe comprises more than one type of molecule (e.g., more than one of a protein, a nucleic acid, a chemical linker or a chemical moiety).

Embodiments of the technology comprise a query probe (e.g., a detectably labeled query probe) that binds transiently and repeatedly to the analyte, e.g., a query probe that binds to and dissociates from the target analyte several (e.g., greater than 1, 2, 3, 4, 5, 6, 7, 8, 9, 10, or more) times per observation window. In some embodiments, the query probe has a dissociation constant (K_{D}) for the analyte of larger than approximately 1 nanomolar (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more nanomolar) under the assay conditions. In some embodiments, the query probe has a binding and/or a dissociation constant for the analyte that is larger than approximately 1 min⁻¹ (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min⁻¹).

The technology is not limited in the query probe. In some embodiments, the query probe is an antibody or antibody fragment. In some embodiments, the query probe is a low-affinity antibody or antibody fragment. In some embodiments, the query probe is a nanobody, a DNA-binding protein or protein domain, a methylation binding domain (MBD), a kinase, a phosphatase, an acetylase, a deacetylase, an enzyme, or a polypeptide. In some embodiments, the query probe is an oligonucleotide that interacts with the target analyte. For example, in some embodiments the query probe is an oligonucleotide that hybridizes to the target analyte to form a duplex that has a melting temperature that is within approximately 10 degrees Celsius of the temperature at which the observations are made (e.g., approximately 7-12 nucleotides for observation that is performed at room temperature). In some embodiments, the query probe is a mononucleotide. In some embodiments, the query probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the query probe is a pharmaceutical agent, e.g., a drug or other bioactive molecule. In some embodiments, the query probe is a metal ion complex. In some embodiments, the query probe is a methyl-binding domain (e.g., MBD1). In some embodiments, the query probe is labeled with a detectable label as described herein. In some embodiments, the query probe is covalently linked to the detectable label. In some embodiments, the query probe is indirectly and/or non-covalently linked and/or associated with the detectable label. In some embodiments, the detectable label is fluorescent.

In some embodiments, the query probe is a mouse monoclonal antibody.

In some embodiments in which the analyte comprises a carbohydrate or polysaccharide, the query probe comprises a carbohydrate-binding protein such as a lectin or a carbohydrate-binding antibody.

As used herein, an “event” refers to an instance of a query probe binding to an analyte or an instance of query probe dissociation from an analyte, e.g., as measured by monitoring a detectable property indicating the binding of a query probe to an analyte and/or the dissociation of a query probe from an analyte.

As used herein, a “capture probe” is any entity (e.g., molecule, biomolecule, etc.) that recognizes an analyte (e.g., binds to an analyte, e.g., binds specifically to an analyte) and links the analyte to a solid support. In exemplary embodiments, the capture probe is a protein that recognizes an analyte. In some other exemplary embodiments, a capture probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, a capture probe is labeled, e.g., with a detectable label such as, e.g., a fluorescent moiety as described herein. In some embodiments, the capture probe comprises more than one type of molecule (e.g., more than one of a protein, a nucleic acid, a chemical linker or a chemical moiety).

Embodiments of the technology comprise capture of an analyte. In some embodiments, the analyte is captured and immobilized. In some embodiments, the analyte is stably attached to a solid support. In some embodiments, the solid support is immobile relative to a bulk liquid phase contacting the solid support. In some embodiments, the solid support is diffusible within a bulk liquid phase contacting the solid support.

In some embodiments, stable attachment of the target analyte to a surface or other solid substrate is provided by a high-affinity or irreversible interaction (e.g., as used herein, an “irreversible interaction” refers to an interaction having a dissociation half-life longer than the observation time, e.g., in some embodiments, a time that is 1 to 10 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer). The technology is not limited in the components and/or methods used for capture of the analyte. For example, the stable attachment is provided by a variety of methods, including but not limited to one or more of the following.

In some embodiments, an analyte is immobilized by a surface-bound capture probe with a dissociation constant (K_{D}) for the analyte smaller than approximately 1 nanomolar (nM) (e.g., less than 1.5, 1.4, 1.3, 1.2, 1.1, 1.0, 0.9, 0.8, 0.7, 0.6, 0.5 nanomolar) and a dissociation rate constant for the analyte that is smaller than approximately 1 min⁻¹ (e.g., less than approximately 1.5, 1.4, 1.3, 1.2, 1.1, 1.0, 0.9, 0.8, 0.7, 0.6, 0.5 min⁻¹). Exemplary surface-bound capture probes include, e.g., an antibody, antibody fragment, nanobody, or other protein; a high-affinity DNA-binding protein or ribonucleoprotein complex such as Cas9, dCas9, Cpf1, transcription factors, or transcription activator-like effector nucleases (TALENs); an oligonucleotide; a small organic molecule; or a metal ion complex.

In some embodiments, an analyte is immobilized by direct noncovalent attachment to a surface (e.g., by interactions between the analyte and the surface, e.g., a glass surface or a nylon, nitrocellulose, or polyvinylidene difluoride membrane).

In some embodiments, an analyte is immobilized by chemical linking (e.g., by a covalent bond) of the analyte to the solid support. In some embodiments, the analyte is chemically linked to the solid support by, e.g., a carbodiimide, a N-hydroxysuccinimide esters (NHS) ester, a maleimide, a haloacetyl group, a hydrazide, or an alkoxyamine In some embodiments, an analyte is immobilized by radiation (e.g., ultraviolet light)-induced cross-linking of the target analyte to the surface and/or to a capture probe attached to the surface. In some embodiments, the capture probe is a rabbit monoclonal antibody. In some embodiments in which the analyte comprises a carbohydrate or polysaccharide, the capture probe comprises a carbohydrate-binding protein such as a lectin or a carbohydrate-binding antibody.

Alternatively, instead of immobilizing the target analyte to a solid support that is relatively stationary with respect to a bulk phase that contacts the solid support as described above, some embodiments provide that the target analyte is associated with a freely diffusing particle that diffuses within the bulk fluid phase contacting the freely diffusing particle. Accordingly, in some embodiments, the target analyte is covalently or noncovalently bound to a freely diffusing substrate. In some embodiments, the freely diffusing substrate is, e.g., a colloidal particle (e.g., a particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)). In some embodiments, the freely diffusing substrate comprises and/or is made of, e.g., polystyrene, silica, dextran, gold, or DNA origami. In some embodiments, the target analyte is associated with a freely diffusing particle that diffuses slowly relative to the diffusion of the query probe, e.g., the target analyte has a diffusion coefficient that is less than approximately 10% (e.g., less than 15, 14, 13, 12, 11, 10.5, 10.4, 10.3, 10.2, 10.1, 10.0, 9.9, 9.8, 9.7, 9.6, 9.5, or 9.0% or less) of the diffusion coefficient of the query probe.

Furthermore, in some embodiments the target analyte is associated with a freely diffusing particle and the location of the target analyte is observable and/or recordable independently of observing and/or recording query probe binding. For example, in some embodiments a detectable label (e.g., a fluorophore, fluorescent protein, quantum dot) is covalently or noncovalently attached to the target analyte, e.g., for detection and localization of the target analyte. Accordingly, in some embodiments the position of the target analyte and the position of query probe binding events are simultaneously and independently measured.

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B})) are used interchangeably for the following binding reaction of A and B at equilibrium:

A+B [symbol] AB

where A and B are two entities that associate with each other (e.g., capture probe and analyte, query probe and analyte) and K_{eq}=[AB]/([A]×[B]). The dissociation constant Ku=1/K_{B}. The K_{D} is a useful way to describe the affinity of a one binding partner A for a partner B with which it associates, e.g., the number K_{D} represents the concentration of A or B that is required to yield a significant amount of AB. K_{eq}=k_{off}/kₒₙ; K_{D}=k_{off}/kₒₙ.

As used herein, a “significant amount” of the product of two entities that associate with each other, e.g., formation of AB from A and B according to the equation above, refers to a concentration of AB that is equal to or greater than the free concentration of A or B, whichever is smaller.

As used herein, “nanomolar affinity range” refers to the association of two components that has an equilibrium dissociation constant K_{D} (e.g., ratio of k_{off}/kₒₙ) in the nanomolar range, e.g., a dissociation constant (Ku) of 1×10⁻¹⁰ to 1×10⁻⁵ M (e.g., in some embodiments 1×10⁻⁹ to 1×10⁻⁶ M). The dissociation constant has molar units (M). The smaller the dissociation constant, the higher the affinity between two components (e.g., capture probe and analyte; query probe and analyte).

As used herein, a “weak affinity” or “weak binding” or “weak association” refers to an association having a K_{D} of approximately 100 nanomolar (e.g., approximately 10, 20, 30, 40, 50, 60, 70, 80, 90, 100, 110, 120, 130, 140, 150, 160, 170, 180, 190, 200, 300, 400, or 500 nanomolar) and/or, in some embodiments, in the range of 1 nanomolar to 10 micromolar.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

As used herein, the word “presence” or “absence” (or, alternatively, “present” or “absent”) is used in a relative sense to describe the amount or level of a particular entity (e.g., an analyte). For example, when an analyte is said to be “present” in a sample, it means the level or amount of this analyte is above a pre-determined threshold; conversely, when an analyte is said to be “absent” in a sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

The term “detection assay” refers to an assay for detecting the presence or absence of an analyte or the activity or effect of an analyte or for detecting the presence or absence of a variant of an analyte.

A “system” denotes a set of components, real or abstract, comprising a whole where each component interacts with or is related to at least one other component within the whole.

In some embodiments the technology comprises an antibody component or moiety, e.g., an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from nonhypervariable or framework regions influence the overall domain structure and hence the combining site. Some embodiments comprise a fragment of an antibody, e.g., any protein or polypeptide-containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Fragments of antibodies include, but are not limited to, Fab (e.g., by papain digestion), F(ab′)2 (e.g., by pepsin digestion), Fab′ (e.g., by pepsin digestion and partial reduction) and Fv or scFv (e.g., by molecular biology techniques) fragments.

A Fab fragment can be obtained by treating an antibody with the protease papaine. Also, the Fab may be produced by inserting DNA encoding a Fab of the antibody into a vector for prokaryotic expression system or for eukaryotic expression system, and introducing the vector into a prokaryote or eukaryote to express the Fab. A F(ab′)₂ may be obtained by treating an antibody with the protease pepsin. Also, the F(ab′)₂ can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote, and introducing the vector into a prokaryote or eukaryote for its expression. A Fv fragment may be produced by restricted cleavage by pepsin, e.g., at 4° C. and pH 4.0. (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein.

As used herein, a “stable interaction” or referring to a “stably bound” interaction refers to an association that is relatively persistent under the thermodynamic equilibrium conditions of the interaction. In some embodiments, a “stable interaction” is an interaction between two components having a K_{D} that is smaller than approximately 10⁻⁹ M or, in some embodiments a K_{D} that is smaller than 10⁻⁸ M. In some embodiments, a “stable interaction” has a dissociation rate constant k_{off} that is smaller than 1 per hour or, in some embodiments, a dissociation rate constant k_{off} that is smaller than 1 per minute. In some embodiments, a “stable interaction” is defined as not being a “transient interaction”. In some embodiments, a “stable interaction” includes interactions mediated by covalent bonds and other interactions that are not typically described by a K_{D} value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} or another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, “moiety” refers to one of two or more parts into which something may be divided, such as, for example, the various parts of an oligonucleotide, a molecule, a chemical group, a domain, a probe, an “R” group, a polypeptide, etc.

As used herein, in some embodiments a “signal” is a time-varying quantity associated with one or more properties of a sample that is assayed, e.g., the binding of a query probe to an analyte and/or dissociation of a query probe from an analyte. A signal can be continuous in the time domain or discrete in the time domain. As a mathematical abstraction, the domain of a continuous-time signal is the set of real numbers (or an interval thereof) and the domain of a discrete-time signal is the set of integers (or an interval thereof). Discrete signals often arise via “digital sampling” of continuous signals. For example, an audio signal consists of a continually fluctuating voltage on a line that can be digitized by reading the voltage level on the line at a regular interval, e.g., every 50 microseconds. The resulting stream of numbers is stored as a discrete-time digital signal. In some embodiments, the signal is recorded as a function of location is space (e.g., x, y coordinates; e.g., x, y, z coordinates). In some embodiments, the signal is recorded as a function of time. In some embodiments, the signal is recorded as a function of time and location.

The term “substantially” as used herein, is a broad term and is used in its ordinary sense, including, but not limited to, being largely but not necessarily wholly that which is specified.

The term “algorithm,” as used herein, is a broad term and is used in its ordinary sense, including, but not limited to, the computational processes (for example, programs) involved in transforming information from one state to another, for example using computer processing.

As used herein, the term “porous medium” or “porous material” refers to a material comprising pores or voids. A porous material has a property of letting only certain substances pass through that are smaller than the pore size, while blocking other substances that are larger than the pore size. A porous medium functions as a molecular, atomic, and/or ionic sieve.

As used herein, the term “nanoporous” refers to a material comprising pores that have a size of 100 nanometers or smaller.

As used herein, the term “microporous” refers to a material comprising pores that have a size of from 0.2 to 2 nm.

As used herein, the term “mesoporous” refers to a material comprising pores that have a size from 2 to 50 nm.

As used herein, the term “macroporous” refers to a material comprising pores than have a size from 50 to 1000 nm.

### Aqueous Two-Phase Systems

Embodiments of the technology relate to the use of aqueous two-phase systems (ATPS) to concentrate an analyte. ATPS have been studied for various preparative and analytical procedures involving, e.g., proteins, nucleic acids, viruses, organelles, and cells since the pioneering work of Albertsson in 1955 (see, e.g., Walter, H. Partitioning In Aqueous Two-Phase System: Theory, Methods, Uses, and Applications To Biotechnology. (Elsevier, 2012), incorporated herein by reference). Since then, many types of ATPS have been described, e.g., ATPS comprising two organic polymers (polymer-polymer systems), a polymer and one or more salts (polymer-salt systems), ionic liquids, short-chain alcohols, micelles or reverse micelles comprising ionic or non-ionic surfactants (see, e.g., Iqbal, M. et al. Aqueous two-phase system (ATPS): an overview and advances in its applications. Biol Proced Online 18, (2016), incorporated herein by reference), and coacervates (see, e.g., Jia, Hentrich, & Szostak, Rapid RNA Exchange in Aqueous Two-Phase System and Coacervate Droplets. Origins of Life and Evolution of Biospheres 44, 1-12 (2014), incorporated herein by reference).

The most common polymer-polymer systems comprise polyethylene glycols (PEG) and dextran, or PEG and dextran sulfate salts (e.g., sodium dextran sulfate), but several others have been described. For instance, ATPS comprising thermoseparating polymers have been reported (see, e.g., Persson, et al. Aqueous polymer two-phase systems formed by new thermoseparating polymers. Bioseparation 9, 105-116 (2000), incorporated herein by reference), e.g., copolymers of ethylene oxide and propylene oxide (EO-PO), poly (N-isopropylacrylamide) (poly-NIPAM), poly vinyl caprolactam (poly-VCL), and copolymers of N-isopropylacrylamide and vinyl caprolactam with vinyl imidazole (poly(NIPAM-VI) and poly(VCL-VI), respectively). Examples of polymer-salt systems include, e.g., PEG and salts such as sodium phosphate, sodium sulfate, ammonium sulfate, sodium citrate (see, e.g., Iqbal, supra), or magnesium sulfate (see, e.g., Mattiasson, B. Applications of aqueous two-phase systems in biotechnology. Trends in Biotechnology 1, 16-20 (1983), incorporated herein by reference), sometimes with other added salts such as sodium chloride or potassium chloride (see, e.g., Gündüz & Korkmaz Bovine serum albumin partitioning in an aqueous two-phase system: Effect of pH and sodium chloride concentration. Journal of Chromatography B: Biomedical Sciences and Applications 743, 255-258 (2000), incorporated herein by reference). An example of a coacervate is an ATP/poly-L-lysine coacervate system, which has been shown to partition short RNA molecules (see, e.g., Jia and Szostak, supra). Systems comprising more than two phases have also been described (see, e.g., Hatti-Kaul, R. Aqueous two-phase systems. Mol Biotechnol 19, 269-277 (2001), incorporated herein by reference).

In polymer-containing ATPS such as PEG-dextran or PEG-salt systems, the phase separation behavior (reflected by the shape and position of the binodal line indicating the boundary between compositions that give rise to one or two phases) can be affected by factors such as pH and polymer molecular weight (see, e.g., Alves et al. Aqueous Two-Phase Systems of Poly(ethylene glycol) and Sodium Citrate: Experimental Results and Modeling. J. Chem. Eng. Data 53, 1587-1594 (2008), incorporated herein by reference), polymer or salt concentration (see, e.g., Azevedo et al. Partitioning of human antibodies in polyethylene glycol-sodium citrate aqueous two-phase systems. Separation and Purification Technology 65, 14-21 (2009), incorporated herein by reference), and temperature (see, e.g., Persson, supra).

In general, a biomolecule or biomolecular complex (including nucleic acids, carbohydrates, proteins, peptides, viruses) dissolved or suspended in a given ATPS will exhibit a different chemical potential in one phase than in the other, resulting in a higher concentration of the biomolecule in one of the two phases at equilibrium (e.g., a partition coefficient greater than or less than one; see, e.g., Iqbal, supra). If a biomolecule within a mixture partitions more strongly into one of the two phases of an ATPS than impurities in the same mixture, the ATPS may be used for purification of the biomolecule away from the impurities. For instance, PEG/dextran, PEG/sodium citrate, and PEG/ammonium sulfate systems have been utilized for the separation of plasmid DNA from complex mixtures (see, e.g., Gomes et al. Purification of plasmid DNA with aqueous two phase systems of PEG 600 and sodium citrate/ammonium sulfate. Separation and Purification Technology 65, 22-30 (2009), incorporated herein by reference) or for separation of single-stranded and double-stranded plasmid DNA (see, e.g., Alberts, B. M. Efficient Separation of Single-Stranded and Double-Stranded Deoxyribonucleic Acid in a Dextran-Polyethylene Glycol Two-Phase System. Biochemistry 6, 2527-2532 (1967), incorporated herein by reference).

ATPS comprising sodium dextran sulfate, methylcellulose, polyvinyl alcohol, and polyethylene glycol were reported to be useful in the purification and concentration of viral particles (see, e.g., Philipson et al. The purification and concentration of viruses by aqueous polymer phase systems. Virology 11, 553-571 (1960), incorporated herein by reference). In some cases, the pH or the concentrations of additives, e.g., salts or affinity ligands, have been shown to impact partitioning of a biomolecule into one of the two phases. For instance, pH and sodium chloride concentration influence the partitioning of bovine serum albumin in a PEG/sodium citrate system (see, e.g., Gündüz, supra). In a PEG/sodium sulfate system, pH, PEG molecular weight, and the concentration of salts such as sodium phosphate and potassium chloride were found to significantly affect the partitioning of a single-stranded fluorescent pyrimidine DNA oligonucleotide (see, e.g., Nazer et al. Partitioning of pyrimidine single stranded oligonucleotide using polyethylene glycol-sodium sulfate aqueous two-phase systems; experimental and modeling. Fluid Phase Equilibria 432, 45-53 (2017), incorporated herein by reference). Covalent modification of one of the ATPS-forming components—such as PEG or dextran polymer—with an affinity ligand has also been employed to induce or enhance partitioning of a biomolecule into one phase, a strategy called affinity partitioning (see, e.g., Walter & Krob Hydrophobic affinity partition in aqueous two-phase systems containing polyethylene glycol)-palmitate of rightside-out and inside-out vesicles from human erythrocyte membranes. FEBS Letters 61, 290-293, incorporated herein by reference).

While ATPS have been extensively investigated for preparative applications such as purification and isolation of natural products, they have rarely been employed for surface-based analytical applications. One exception is the reported use of a PEG-dextran system to trap detection reagents on discrete regions of a surface to prevent cross-reactivity in multiplexed ELISA (see, e.g., Frampton, J. P. et al. Aqueous two-phase system patterning of detection antibody solutions for cross-reaction-free multiplex ELISA. Scientific Reports 4, 4878 (2014); Simon, A., Frampton, J., White, J. & Takayama, S. Systems and methods for multiplex solution assays. (2014), incorporated herein by reference). A PEG/dextran ATPS was reported to increase the sensitivity of a polymerase chain reaction (PCR) assay for Listeria monocytogenes in soft cheese by partitioning of PCR inhibitors to a different phase than the nucleic acid analyte (see, e.g., Lantz et al. Enhanced sensitivity in PCR detection of Listeria monocytogenes in soft cheese through use of an aqueous two-phase system as a sample preparation method. Appl. Environ. Microbiol. 60, 3416-3418 (1994), incorporated herein by reference); similar results were reported for the use of a PEG/dextran system as a preparative step for PCR detection of Helicobacter pylori in human feces (see, e.g., id). Other reported analytical applications include investigation of protein heterogeneity, interactions between proteins or between proteins and low-molecular-weight compounds, and the effect of pH on physicochemical properties of proteins (see, e.g., Walter, H. Partitioning In Aqueous Two-Phase System: Theory, Methods, Uses, and Applications To Biotechnology. (Elsevier, 2012), incorporated herein by reference).

Although the capacity of ATPS to concentrate a biomolecule, cell, viral particle, or organelle in one of two phases (typically the phase of smaller volume) has been noted (Iqbal, supra), prior to the development of the technology described herein, it was unclear whether ATPS would provide an effective strategy for increasing the sensitivity of surface-based biomolecule assays by a significant factor (e.g., greater than 10-fold) for biomolecular analytes in general, or for nucleic acids in particular. For example, it was unclear whether the effects of high ionic strength (e.g., greater than 10% sodium citrate by weight) or the high viscosity of polymer-rich phases (e.g., greater than 10% PEG or greater than 5% dextran by weight) might negatively impact the surface capture of an analyte in a surface-based assay, e.g., by denaturing biochemical components involved in analyte capture, by inducing aggregation of the analyte, by reducing the affinity of the analyte for a capture ligand, or by slowing diffusion of the analyte to the surface for capture.

In contrast to previous technologies, experiments conducted during the development of embodiments of the technology provided herein indicated that the sensitivity of surface-based single-molecule assays is improved by more than an order of magnitude (e.g., approximately 10- to 50-fold) by using a pre-concentration step using ATPS that places the analyte-rich phase in contact with a surface for analyte capture, especially if the analyte solution is added to a lyophilized (dry) mixture of the ATPS-forming components to limit the overall volume of the final mixture, thus increasing the factor by which the analyte is concentrated compared to the original mixture. Further, data collected during these experiments indicated that ATPS comprising PEG, sodium citrate, and sodium chloride were particularly effective for increasing sensitivity of surface-based assays for small nucleic acids including microRNAs and short DNA fragments (e.g., less than or equal to 160 base pairs) due to their high partition coefficients (e.g., >1000), relatively low viscosity of the analyte-rich phase, and compatibility with efficient surface capture. Experiments conducted during the development of embodiments of the technology indicated that, given the variety of ATPS that have been described and the diversity of analytes that can be partitioned using ATPS, the technology contemplates several other ATPS compositions that are useful for surface-based assays. Accordingly, ATPS can be chosen from previously described systems to maximize the partitioning and surface capture of a given analyte. Common characteristics of useful ATPS systems included within the technology described herein are ATPS that give rise to a partition coefficient less than 0.1 or greater than 10 for the analyte of interest, e.g., because as they yield the largest potential concentration factors for the analyte.

As noted above, ATPS compositions have been shown to partition DNA selectively, including polyethylene glycol (PEG)/sodium citrate (SC) (see, e.g., Gomes et al. (2009) “Purification of plasmid DNA with aqueous two phase systems of PEG 600 and sodium citrate/ammonium sulfate” Sep. Purif. Technol. 65: 22-30, incorporated herein by reference in its entirety), PEG/sodium sulfate (see, e.g., Nazer et al. (2017) “Partitioning of pyrimidine single stranded oligonucleotide using polyethylene glycol-sodium sulfate aqueous two-phase systems; experimental and modeling” Fluid Phase Equilibria 432: 45-53), and PEG/dextran (see, e.g., Alberts, supra, incorporated herein by reference in its entirety). While plasmid DNA has been shown to exhibit as much as a 100-fold to 1000-fold selectivity for one phase over the other (see, e.g., Alberts, supra; see, e.g., Nazer, supra, each of which is incorporated herein by reference in its entirety), short nucleic acids (e.g., DNA fragments found in biofluids) were not expected to exhibit equally high partition coefficients into a phase of a ATPS.

While aqueous two-phase systems have been previously described, the technology described herein relates to the use of ATPS to analyze rare and/or dilute analytes (e.g., tumor DNA) in bulk biofluids (such as urine). Further, the technology relates to using ATPS for transporting very dilute analytes to a surface for immobilization and analysis (e.g., by single-molecule techniques such as SiMREPS). In particular, embodiments of the technology provide use of sodium chloride in a PEG/sodium citrate ATPS to enhance the partitioning of nucleic acids (e.g., low-molecular-weight DNA (e.g., DNA fragments comprising less than 200 base pairs or nucleotides (e.g., less than 200, 190, 180, 170, 160, 150, 140, 130, 120, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, or fewer than 10 nt or bp))) into the salt-rich phase (e.g., with a partition coefficient greater than 100). Experiments were conducted during the development of the technology described herein indicating that the technology provides approximately 100-fold concentration of DNA with retention of at least 50% of the analyte. Furthermore, embodiments of the technology provide concentration of analyte (e.g., nucleic acids) by ATPS in less than 5 minutes.

By comparison, alcohol-mediated (e.g., ethanol- or butanol-mediated) precipitation of DNA is more labor-intensive and time-consuming. In particular, a standard ethanol precipitation of DNA comprises greater than 2 hours (for precipitation, centrifugation, and resuspension of DNA) and yields crystalline solids that can damage solid supports by abrasion, in addition to requiring the use of organic solvents that are not compatible with many surface chemistries intended for use in aqueous solutions. Further, extraction of DNA using column methods (e.g., binding to and elution from Q-SEPHAROSE resin) has been used to analyze tumor DNA from urine, but these existing column technologies are more labor-intensive and time-consuming than the ATPS technology described herein and does not readily permit concentration of analytes into volumes of only a few microliters.

In some embodiments, the technology comprises use of an ATPS as described in U.S. provisional patent application Ser. No. 62/598,802, filed Dec. 14, 2017, which is incorporated herein by reference in its entirety.

### SiMREPS

As used herein, the term “single-molecule recognition through equilibrium Poisson sampling” and its abbreviation “SiMREPS” refers to an amplification-free, single-molecule detection approach for identifying and counting individual microRNA molecules in biofluids by kinetic fingerprinting. The technology is described in, e.g., U.S. patent application Ser. No. 14/589,467; Int'l Pat. App. No. PCT/US2015/044650; Int'l Pat. App. No. PCT/US2017/016977; and U.S. Provisional App. Ser. No. 62/468,578; each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In brief, the SiMREPS technology comprises directly observing the repeated binding of fluorescent probes to surface-captured analytes (e.g., nucleic acid, protein, etc.), which produces a specific (e.g., for nucleic acid, a sequence-specific) kinetic fingerprint. The kinetic fingerprint identifies the analyte with high-confidence at single-molecule resolution. The kinetic fingerprint overcomes previous technologies limited by thermodynamic specificity barriers and thereby minimizes and/or eliminates false positives. Thus, the SiMREPS technology provides an ultra-high specificity that finds use in detecting, e.g., rare analytes such as rare or low-abundance mutant DNA alleles. Prior work has shown that SiMREPS is capable of single-nucleotide discrimination (see, e.g., Johnson-Buck et al. (2015) “Kinetic fingerprinting to identify and count single nucleic acids” Nat. Biotechnol. 33: 730-32; Su et al. (2017) “Single-Molecule Counting of Point Mutations by Transient DNA Binding” Sci Rep 7: 43824, each of which is incorporated herein by reference).

The technology provides for the detection of target analytes, e.g., in the presence of similar analytes and, in some embodiments, background noise. In some embodiments, signal originating from the transient binding of the query probe to the target analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the target analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the target analyte and/or a fluorescence emission that is dequenched when bound to the target analyte.

The technology comprises locating and/or observing the transient binding of a query probe to an analyte within a discrete region of an area and/or a discreet region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) target analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) target analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence. Consequently, in some embodiments detection of bound query probes comprises use of total internal reflection fluorescence microscopy (TIRF), HiLo microscopy (see, e.g., US20090084980, EP2300983 B1, WO2014018584 A1, WO2014018584 A1, incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized target analyte. Binding of the query probe to the target analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of a target analyte). In some embodiments, a binding event has a fluorescence intensity that is at least 1, 2, 3, 4 or more standard deviations above the background fluorescence intensity (e.g., the fluorescence intensity observed in the absence of a target analyte). In some embodiments, a binding event has a fluorescence intensity that is at least 2 standard deviations above the background fluorescence intensity (e.g., the fluorescence intensity observed in the absence of a target analyte). In some embodiments, a binding event has a fluorescence intensity that is at least 1.5, 2, 3, 4, or 5 times the background fluorescence intensity (e.g., the mean fluorescence intensity observed in the absence of a target analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where a target analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the target analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the target analyte (e.g., an on/off event), e.g., a query probe dissociating from a bound state or a query probe associating with a target analyte from the unbound state.

Methods according to the technology comprise counting the number of query probe binding events that occur at each discrete location (e.g., at a position identified by x, y coordinates) on the solid support during a defined time interval that is the “acquisition time” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 0 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes).

Further, the length of time the query probe remains bound to the target analyte during a binding event is the “dwell time” of the binding event. The number of binding events detected during the acquisition time and/or the lengths of the dwell times recorded for the binding events is/are characteristic of a query probe binding to a target analyte and thus provide an indication that the target analyte is immobilized at said discrete location and thus that the target analyte is present in the sample.

Binding of the query probe to the immobilized target analyte and/or and dissociation of the query probe from the immobilized target analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the nucleic acid during the acquisition time and/or the length of time the query probe remains bound to the nucleic acid during each binding event and the length of time the query probe remains unbound to the nucleic acid between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

In some embodiments, data comprising measurements of fluorescence intensity at the emission wavelength of the query probe are recorded as a function of time. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of one or more query probes) are counted for each discrete location on the solid support where a target analyte is immobilized. In some embodiments, a threshold number of transitions is used to discriminate the presence of a target analyte at a discrete location on the solid support from background signal, non-target analyte, and/or spurious binding of the query probe.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes.

In some embodiments, the distribution produced from a target analyte is significantly different than a distribution produced from a non-target analyte or the distribution produced in the absence of a target analyte. In some embodiments, a mean number of transitions is determined for the plurality of immobilized target analytes. In some embodiments, the mean number of transitions observed for a sample comprising a target analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of a target analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of a target analyte at said discrete location on the solid support.

In some embodiments, dwell times of bound query probe (τₒₙ) and unbound query probe (τ_{off}) are used to identify the presence of a target analyte in a sample and/or to distinguish a sample comprising a target analyte from a sample comprising a non-target analyte and/or not comprising the target analyte. For example, the τₒₙ for a target analyte is greater than the τₒₙ for a non-target analyte; and, the τ_{off} for a target analyte is smaller than the τ_{off} for a non-target analyte. In some embodiments, measuring τₒₙ and τ_{off} for a negative control and for a sample indicates the presence or absence of the target analyte in the sample. In some embodiments, a plurality of τₒₙ and τ_{off} values is determined for each of a plurality of spots imaged on a solid support, e.g., for a control (e.g., positive and/or negative control) and a sample suspected of comprising a target analyte. In some embodiments, a mean τₒₙ and/or τ_{off} is determined for each of a plurality of spots imaged on a solid support, e.g., for a control (e.g., positive and/or negative control) and a sample suspected of comprising a target analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of the target analyte in the sample.

As described herein, the technology detects analytes by a kinetic detection technology. Accordingly, particular embodiments of the technology are related to detecting an analyte by analyzing the kinetics of the interaction of a query probe with the analyte to be detected. For the interaction of a query probe Q (e.g., at an equilibrium concentration [Q]) with a target analyte T (e.g., at an equilibrium concentration [T]), the kinetic rate constant kₒₙ describes the time-dependent formation of the complex QT comprising the probe Q hybridized to the analyte T. In particular embodiments, while the formation of the QT complex is associated with a second order rate constant that is dependent on the concentration of query probe and has units of M⁻¹ min⁻¹ (or the like), the formation of the QT complex is sufficiently described by a kₒₙ that is a pseudo-first order rate constant associated with the formation of the QT complex. Thus, as used herein, kₒₙ is an apparent (“pseudo”) first-order rate constant.

Likewise, the kinetic rate constant k_{off} describes the time-dependent dissociation of the complex QT into the probe Q and the analyte T. Kinetic rates are typically provided herein in units of min⁻¹ or s⁻¹. The “dwell time” of the query probe Q in the bound state (τₒₙ) is the time interval (e.g., length of time) that the probe Q is hybridized to the analyte T during each instance of query probe Q binding to the analyte T to form the QT complex. The “dwell time” of the query probe Q in the unbound state (τ_{off}) is the time interval (e.g., length of time) that the probe Q is not hybridized to the analyte T between each instance of query probe Q binding to the analyte to form the QT complex (e.g., the time the query probe Q is dissociated from the target analyte T between successive binding events of the query probe Q to the target analyte T). Dwell times may be provided as averages or weighted averages integrating over numerous binding and non-binding events.

Further, in some embodiments, the repeated, stochastic binding of probes (e.g., detectably labeled query probes (e.g., fluorescent probes) to target analytes is modeled as a Poisson process occurring with constant probability per unit time and in which the standard deviation in the number of binding and dissociation events per unit time (N_{b+d}) increases as (N_{b+d})^{1/2}. Thus, the statistical noise becomes a smaller fraction of N_{b+d} as the observation time is increased. Accordingly, the observation is lengthened as needed in some embodiments to achieve discrimination between target and off-target binding. And, as the acquisition time is increased, the signal and background peaks in the N_{b+d} histogram become increasingly separated and the width of the signal distribution increases as the square root of N_{b+d}, consistent with kinetic Monte Carlo simulations.

Further, in some embodiments assay conditions are controlled to tune the kinetic behavior to improve discrimination of query probe binding events to the target analyte from background binding. For example, in some embodiments the technology comprises control of assay conditions such as, e.g., using a query probe that is designed to interact weakly with the target analyte (e.g., in the nanomolar affinity range); controlling the temperature such that the query probe interacts weakly with the target analyte; controlling the solution conditions, e.g., ionic strength, ionic composition, addition of chaotropic agents, and addition of competing probes.

Some embodiments provide a method of identifying an analyte by repetitive query probe binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non-covalent interaction between the solid support and analyte. In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the target analyte. In some embodiments, methods comprise detecting the repetitive (e.g., transient, low-affinity) binding of a query probe to the target analyte. In some embodiments, methods comprise generating a dataset comprising a signal produced from query probe binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in particular embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame subtraction process to generate differential intensity profiles showing query probe binding or dissociation events within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given target analyte.

For instance, some embodiments of methods for quantifying one or more surface-immobilized or diffusing target analytes comprise one or more steps including, e.g., measuring the signal of one or more transiently binding query probes to the immobilized target analyte(s) with single-molecule sensitivity. In some embodiments, methods comprise tracking (e.g., detecting and/or recording the position of) target analytes independently from query probe binding. In some embodiments, the methods further comprise calculating the time-dependent probe binding signal intensity changes at the surface as a function of position (e.g., x, y position). In some embodiments, calculating the time-dependent query probe binding signal intensity changes at the surface as a function of position (e.g., x, y position) produces a “differential intensity profile” for query probe binding to the analyte. In some embodiments, the methods comprise determining the position (e.g., x, y position) of each query probe binding and dissociation event (“event”) with sub-pixel accuracy from a differential intensity profile. In some embodiments, methods comprise grouping events into local clusters by position (e.g., x, y position) on the surface, e.g., to associate events for a single immobilized target analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the target analyte is a nucleic acid. In some embodiments, the target analyte is a small molecule.

In some embodiments, the interaction between the target analyte and the query probe is distinguishably influenced by a covalent modification of the target analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, the analyte is a nucleic acid comprising a covalent modification to a nucleobase, a ribose, or a deoxyribose moiety of the target analyte.

In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer.

In some embodiments, the query probe is a low-affinity antibody, antibody fragment, or nanobody.

In some embodiments, the query probe is a DNA-binding protein, RNA-binding protein, or a DNA-binding ribonucleoprotein complex.

In some embodiments, the position, e.g., the (x,y) position, of each binding or dissociation event is determined by subjecting the differential intensity profile to centroid determination, least-squares fitting to a Gaussian function, least-square fitting to an airy disk function, least-squares fitting to a polynomial function (e.g., a parabola), or maximum likelihood estimation.

In some embodiments, the capture probe is a high-affinity antibody, antibody fragment, or nanobody. In some embodiments, the capture probe is a nucleic acid. In some embodiments, capture is mediated by a covalent bond cross-linking the target analyte to the surface. In some embodiments, the target analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### ATPS Methods and Compositions

In some embodiments, the technology provides a method for analysis of an analyte (e.g., a biomolecular analyte), e.g., from a biologically derived mixture (e.g., a biofluid or other sample). For instance, in some embodiments, the technology provides a method comprising adding an analyte-containing composition (e.g., a biofluid or sample) to an aqueous two-phase system (ATPS) (e.g., a mixture of materials that forms an aqueous two-phase system upon mixing). In some embodiments, the ATPS is a solid-phase ATPS. In some embodiments, the ATPS is neat. In some embodiments, the analyte is concentrated into one of the two phases of the ATPS. For example, in some embodiments the analyte is concentrated at least 10-fold (e.g., at least 10, 15, 20, 25, 30, 35, 40, 45, 50, 55, 60, 65, 70, 75, 80, 85, 90, 90, 100, 150, 200, 250, 300, 350, 400, 450, 500, 550, 600, 650, 700, 750, 800, 850, 900, 950, or 100-fold or more) relative to its concentration prior to formation of the ATPS. In some embodiments, the two phases have different volumes, e.g., in some embodiments one phase has a volume that is 2, 3, 4, 5, 6, 7, 8, 9, 10, 11, 12, 13, 14, 15, 16, 17, 18, 19, 20, 30, 40, 50, or 100, or more times the volume of the second phase of the system.

In some embodiments, the analyte preferentially partitions into the phase having the smaller volume; thus, in some embodiments, the analyte is concentrated into the phase having the smaller volume. In some embodiments, methods comprise separating the two phases (e.g., by applying a force (e.g., gravity or centrifugation) to the ATPS). In some embodiments, centrifugation is carried out at approximately 1000×g for 10-120 minutes; in some embodiments, centrifugation is carried out at approximately 1000×g for approximately 1 minute. In some embodiments, centrifugation is carried out at approximately 1000×g (e.g., at 500, 600, 700, 800, 850, 900, 925, 950, 975, 1000, 1025, 1050, 1075, 1100, 1150, 1200, 1250, 1300, 1400, or 1500×g) for approximately 1 to 120 minutes (e.g., 0.5, 0.75, 1, 1.25, 1.5, 1.75, 2, 2.25, 2.5, 2.75, 3.0, 3.25, 3.5, 3.75, 4, 4.25, 4.5, 4.75, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 85, 90, 95, 100, 105, 110, 115, or 120 minutes).

In some embodiments, the methods comprise capturing the analyte from the analyte-rich phase by contacting the analyte-rich phase to a surface. For example, in some embodiments the technology comprises contacting the surface with the analyte-rich phase for greater than 1 minute (e.g., more than 1, 2, 3, 4, 5, 6, 7, 8, 9, 10, 20, 30, 40, 60, or 120 minutes). In some embodiments, contact is maintained between the analyte-rich phase and the surface by contacting the surface with the ATPS while a force is applied to the ATPS to separate the phases such that the analyte-rich phase is produced (e.g., and separated from the analyte-poor phase) and contacts the surface. That is, in some embodiments the surface contacts the ATPS (e.g., contacts the analyte-rich phase of the ATPS) during the centrifuging or separation of phases by gravity. In some embodiments, methods comprise detecting the analyte at the solid-phase substrate. In some embodiments, detecting the analyte comprises use of a single-molecule detection method. In some embodiments, detecting the analyte comprises analyzing the kinetics of binding and dissociation of a detectably labeled query probe to the analyte with single-molecule sensitivity (see, e.g., U.S. patent application Ser. No. 14/589,467; Int'l Pat. App. No. PCT/US2015/044650; Int'l Pat. App. No. PCT/US2017/016977; and U.S. Provisional App. Ser. No. 62/468,578; each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In some embodiments, the technology provides methods for the analysis of a nucleic acid. In some embodiments, the technology provides methods for the analysis of a nucleic acid comprising fewer than 200 base pairs or nucleotides (e.g., comprising fewer than 200, 190, 180, 170, 160, 150, 140, 130, 120, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, or fewer than 10 nt or bp). In some embodiments, the technology provides methods for analyzing nucleic acids in or from a sample (e.g., a biological sample (e.g., a biofluid)). In some embodiments, the technology provides methods for analyzing nucleic acids derived from a biofluid, cell lysate, lysate of a single cell, lysate of a single subcellular compartment. For example, in some embodiments methods comprise adding a sample (e.g., a biological sample (e.g., a biofluid)) comprising a nucleic acid to an aqueous two-phase system (ATPS) (e.g., a mixture of materials that forms an aqueous two-phase system upon mixing (e.g., a composition comprising polyethylene glycol (PEG), sodium citrate/citric acid, and a second salt (e.g., sodium chloride, sodium sulfate, lithium chloride, potassium chloride, or magnesium chloride))). In some embodiments, the second salt improves the partition coefficient of the nucleic acid into the citrate-rich phase relative to the partition coefficient of the nucleic acid into the citrate-rich phase without the second salt. Furthermore, embodiments comprise mixing the ATPS composition (e.g., in some embodiments, to dissolve a solid mixture completely) and forming two aqueous phases.

In some embodiments, methods comprise separating the two phases (e.g., by gravity, centrifugation, or by a similar force) to concentrate the nucleic acid in one of the two phases. In some embodiments, the nucleic acid is concentrated in the sodium citrate-rich phase. In some embodiments, the nucleic acid is concentrated at least 10-fold (e.g., at least 10, 15, 20, 25, 30, 35, 40, 45, 50, 55, 60, 65, 70, 75, 80, 85, 90, 90, 100, 150, 200, 250, 300, 350, 400, 450, 500, 550, 600, 650, 700, 750, 800, 850, 900, 950, or 1000-fold or more) in one of the phases of the ATPS. In some embodiments, the two phases have different volumes, e.g., in some embodiments one phase has a volume that is 2, 3, 4, 5, 6, 7, 8, 9, 10, 11, 12, 13, 14, 15, 16, 17, 18, 19, 20, 30, 40, 50, or 100, or more times the volume of the second phase of the system. In some embodiments, the nucleic acid preferentially partitions into the phase having the smaller volume; thus, in some embodiments, the nucleic acid is concentrated into the phase having the smaller volume. Furthermore, in some embodiments the methods comprise capturing the nucleic acid from a phase of the ATPS (e.g., the citrate-rich phase, the phase having the smaller volume) at the surface of a solid-phase substrate. In some embodiments, methods comprise separating the two phases (e.g., by applying a force (e.g., gravity or centrifugation) to the ATPS). In some embodiments, the methods comprise capturing the nucleic acid from the nucleic acid-rich phase (e.g., the citrate-rich phase) by contacting the nucleic acid-rich phase (e.g., the citrate-rich phase) to a surface. For example, in some embodiments the technology comprises contacting the surface with the nucleic acid-rich phase (e.g., the citrate-rich phase) for greater than 1 minute (e.g., more than 1, 2, 3, 4, 5, 6, 7, 8, 9, 10, 20, 30, 60, or 120 minutes). In some embodiments, contact is maintained between the nucleic acid-rich phase (e.g., the citrate-rich phase) and the surface by contacting the surface with the ATPS while a force is applied to the ATPS to separate the phases such that the nucleic acid-rich phase (e.g., the citrate-rich phase) is produced (e.g., and separated from the nucleic acid-poor phase) and contacts the surface. That is, in some embodiments the surface contacts the ATPS (e.g., contacts the nucleic acid-rich phase (e.g., the citrate-rich phase) of the ATPS) during the centrifuging or separation of phases by gravity. In some embodiments, methods comprise detecting the nucleic acid at the solid-phase substrate. In some embodiments, detecting the nucleic acid comprises use of a single-molecule detection method. In some embodiments, detecting the nucleic acid comprises analyzing the kinetics of binding and dissociation of a detectably labeled query probe to the nucleic acid with single-molecule sensitivity (see, e.g., U.S. patent application Ser. No. 14/589,467; Int'l Pat. App. No. PCT/US2015/044650; Int'l Pat. App. No. PCT/US2017/016977; and U.S. Provisional App. Ser. No. 62/468,578; each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In some embodiments, the technology comprises a denaturing step. For example, in some embodiments of the methods, a sample is heated to a temperature that denatures a biomolecule analyte in the sample. In some embodiments, the denaturing step occurs prior to the step of mixing the sample with the ATPS. In some embodiments, the denaturing step occurs after the step of mixing the sample with the ATPS. In some embodiments, the denaturing step comprises heating the sample (e.g., heating to a temperature of at least 40, 45, 50, 55, 60, 65, 70, 75, 80, 85, 90, 95, 100, 105, or 110° C.). In some embodiments, the sample is treated with a chemical denaturant.

In some embodiments, denaturing (e.g., thermal denaturing) occurs in the presence of a carrier oligonucleotide or one or more oligonucleotides that prevent(s) formation of secondary structure or other hybridization of the target to other nucleic acids in the mixture. Accordingly, some embodiments comprise adding a carrier oligonucleotide or one or more oligonucleotides, e.g., to prevent(s) formation of secondary structure or other hybridization of the target to other nucleic acids in the mixture. In some embodiments, the carrier oligonucleotide or one or more oligonucleotides that prevent(s) formation of secondary structure or other hybridization of the target to other nucleic acids in the mixture is added to the sample; in some embodiments, the carrier oligonucleotide or one or more oligonucleotides that prevent(s) formation of secondary structure or other hybridization of the target to other nucleic acids in the mixture is added to the ATPS; in some embodiments, the carrier oligonucleotide or one or more oligonucleotides that prevent(s) formation of secondary structure or other hybridization of the target to other nucleic acids in the mixture is added to a composition comprising the sample and ATPS.

In some embodiments, an enzyme (e.g., a protease (e.g., proteinase K, peptide hydrolase), a lipase) is added prior to denaturing (e.g., prior to thermal denaturing). In some embodiments, the enzyme is added to the sample; in some embodiments, the enzyme is added to the ATPS; in some embodiments, the enzyme is added to a composition comprising the sample and ATPS.

In some embodiments, a detergent is added prior to denaturing (e.g., a detergent including but not limited to SDS (e.g., 0-5%), Triton X-100, Triton X-114, deoxycholate, Brij-35, Brij-58, Tween 20, Tween 80, octyl glucoside, octyl thioglucoside, CHAPS, CHAPSO, etc.) In some embodiments, the detergent is added to the sample; in some embodiments, the detergent is added to the ATPS; in some embodiments, the detergent is added to a composition comprising the sample and ATPS.

In some embodiments, the sample comprises cells (e.g., isolated single cells) or subcellular components (e.g., isolated subcellular components), e.g., mitochondria, nuclei, exosomes, extracellular vesicles, etc. In some embodiments, cells or subcellular components are isolated by a microfluidic technique, a liquid-in-liquid emulsion technique, a micropipet manipulation technique, or other technology known in the art.

In some embodiments, the technology relates to a composition comprising two or more aqueous solutions that form an ATPS, e.g., when the two or more aqueous solutions are mixed. In some embodiments, the technology relates to a composition comprising PEG and a citrate buffer (e.g., citric acid and a citrate salt). In some embodiments, the technology relates to a composition comprising PEG, a citrate buffer (e.g., citric acid and a citrate salt), and a second salt (e.g., sodium chloride). In some embodiments, the technology provides a composition comprising PEG having a mass fraction in the composition that is greater than 30% (e.g., greater than 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 70, 71, 72, 73, 74, or 75%). In some embodiments, the technology provides a composition comprising citrate having a mass fraction in the composition of less than 2.5% (e.g., less than 1.00, 1.25, 1.50, 1.75, 2.00, 2.25, or 2.50%). In some embodiments, the technology provides a composition comprising sodium chloride at a concentration of more than 0.25 M (e.g., more than 0.25, 0.3, 0.35, 0.4, 0.45, 0.5, 0.55, 0.6, 0.65, 0.7, 0.75, 0.8, 0.85, 0.9, 1.0, 1.1, 1.2, 1.3, 1.4, or more than 1.5 M). In some embodiments, the PEG has mean molecular weight in the composition of between 600 and 8000 Da (e.g., 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 7000, or 8000 Da). In some embodiments, the ratio of citric acid to sodium citrate in the composition provides a pH between 5.5 and 8.5 (e.g., a pH of 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5).

In some embodiments, the technology comprises use of an ATPS in a method as described in U.S. provisional patent application Ser. No. 62/598,802, filed Dec. 14, 2017, which is incorporated herein by reference in its entirety.

### Electrophoretic Cells

In some embodiments, the technology relates to methods, devices, and systems for concentrating analytes (e.g., dilute analytes) comprising a net positive or negative electronic charge (including, but not limited to, charged biomolecules such as nucleic acids, proteins, metabolites, and lipids) over a small surface area, e.g., for subsequent immobilization and analysis by a suitable technique (e.g., single molecule recognition by equilibrium Poisson sampling, SiMREPS, as described herein). This method finds use, for example, in applications for sensitive detection of very low concentrations of analyte, but where only a limited region of an imaging surface may be analyzed at a time.

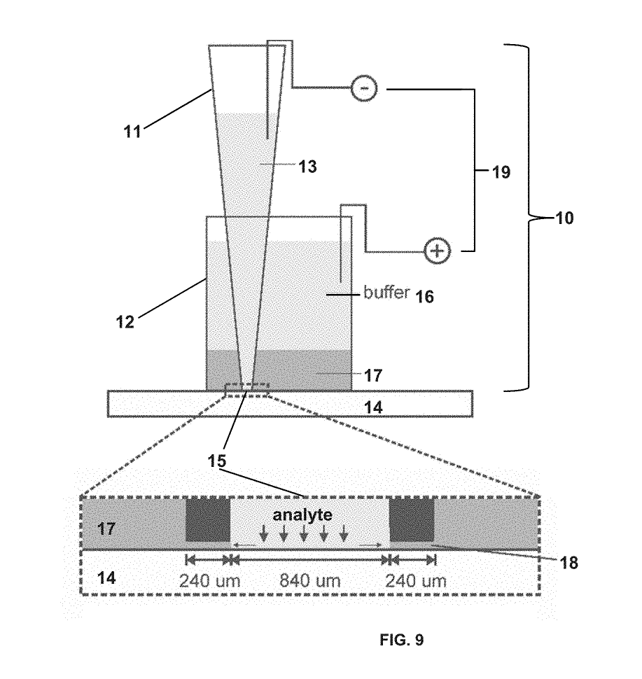

In some embodiments, an analyte is delivered to a surface, contacted to a surface, concentrated at a surface, and/or immobilized at a surface using an electrophoretic cell. In some embodiments, the electrophoretic cell (10) comprises an inner chamber (11) and an outer chamber (12) (see, e.g., FIG. 9). The inner chamber is capable of holding a solution comprising an analyte (13). The outer chamber is capable of holding a buffer (16) (e.g., a solution comprising an electrolyte (e.g., ions (e.g., cations, anions))). The inner chamber (11) comprises a wall surrounding an internal volume. The wall physically and electrically separates the internal volume from the buffer in the outer chamber (12). Accordingly, in some embodiments the wall is an insulator (e.g., a non-conductor). In some embodiments, the inner chamber (11) comprises an opening (15) that places the solution comprising the analyte in contact with a surface (14). In some embodiments, the opening (15) of the inner chamber (11) has a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm). Accordingly, in some embodiments the composition comprising the analyte (13) contacts the surface (14) over an area having a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm; e.g., over an area of less than approximately 100 mm², e.g., less than 100, 95, 90, 85, 80, 75, 70, 65, 60, 55, 50, 45, 40, 35, 30, 25, 20, 18, 16, 14, 12, 10, 9, 8, 7, 6, 5, 4, 3, 2, 1, 0.8, 0.7, 0.6, 0.5, 0.4, 0.3, 0.2, 0.1, 0.08, 0.06, 0.05, 0.02, or 0.01 mm²).

In some embodiments, the outer chamber (12) comprises a base (17). In some embodiments, the base (17) comprises a porous material (e.g., nanoporous, microporous, mesoporous) that allows passage of electrolytes between the inner chamber (11) and outer chamber (12) and minimizes and/or eliminates flow of analyte between the inner chamber (11) and outer chamber (12). In some embodiments, the base (17) comprises a porous material that is a polymer. In some embodiments, the base (17) comprises a porous material that is a polyacrylamide. Optionally, in some embodiments, the pores of the base (17) are blocked with a suitable material (e.g., a branched polymer (e.g., dextran sulfate)) to minimize or eliminate the migration of the analyte into the outer chamber base (17). The technology is not limited to any particular material for the base (17) provided it is a porous material comprising pore sizes that are small enough to exclude the analyte while being large and contiguous enough to permit flow of current (e.g., electrolytes) between the inner chamber (11) and the outer chamber (12).

In some embodiments, the wall of the inner chamber (11) does not contact the surface (14). Accordingly, in some embodiments, the electrophoretic cell (10) comprises a gap (18) between the wall of the inner chamber (11) and the surface (14). In some embodiments, the wall of the inner chamber (11) is separated from the surface (14) by a distance of approximately 10 to 100 μm (e.g., 10, 15, 20, 25, 30, 35, 40, 45, 50, 55, 60, 65, 70, 75, 80, 85, 90, 95, or 100 μm) to provide a gap (18) between the wall of the inner chamber (11) and the surface (14) (see, e.g., FIG. 9). In some embodiments, the gap (18) allows current to flow between the inner chamber (11) and the outer chamber (12) (see, e.g., FIG. 9). In some embodiments, the gap (18) comprises the same material as the base (17).

In some embodiments, the electrophoretic cell (10) comprises an anode and a cathode. In some embodiments, the anode and cathode provide a voltage (e.g., a potential) (19) across the inner chamber (11) and the outer chamber (12). In some embodiments, the anode is electrically connected to the inner chamber (11) and the cathode is electrically connected to the outer chamber (12) (e.g., to move a negatively charged analyte to the surface (14)). In some embodiments, the cathode is electrically connected to the inner chamber (11) and the anode is electrically connected to the outer chamber (12) (e.g., to move a positively charged analyte to the surface (14)).

Related embodiments provide methods for moving an analyte to a surface and/or concentrating an analyte to a surface. For example, methods comprise one or more steps as follows: providing an electrophoretic cell as described herein, providing a surface (e.g., upon which to immobilize and/or concentrate an analyte), providing a solution comprising an analyte (e.g., a sample), placing a solution comprising an analyte in the inner chamber of the electrophoretic cell, contacting the solution comprising the analyte to a surface, providing a buffer comprising an electrolyte, placing a buffer comprising an electrolyte in the outer chamber of the electrophoretic cell, and providing a voltage (e.g., a potential) across the inner and outer chamber. Some embodiments comprise providing a voltage using a direct current (DC) source. Some embodiments comprise providing a voltage using an alternating current (AC) source. In some embodiments, the voltage alternates between approximately 0 to 200 V (e.g., from 0 to 10, 20, 30, 40, 50, 60, 70, 80, 90, 100, 110, 120, 130, 140, 150, 160, 170, 180, 190, or 200 V). In some embodiments, the voltage is constant and is in a range of from 10-200 V (e.g., 10, 20, 30, 40, 50, 60, 70, 80, 90, 100, 110, 120, 130, 140, 150, 160, 170, 180, 190, or 200 V). In some embodiments, the voltage varies with a period of approximately 0.1 to 10 minutes (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.1, 3.2, 3.3, 3.4, 3.5, 3.6, 3.7, 3.8, 3.9, 4.0, 4.5, 5.0, 5.5, 6.0, 6.5, 7.0, 7.5, 8.0, 8.5, 9.0, 9.5, or 10.0 minutes). Further, some embodiments comprise imaging the analyte at the surface, quantifying the analyte at the surface (e.g., by a kinetic detection method as described herein), or determining a concentration of the analyte at the surface. In some embodiments, the analyte is immobilized at the surface as described herein (e.g., by capture with a capture probe as described herein). Some embodiments comprise producing the inner chamber and/or outer chamber, e.g., by extrusion molding, three-dimensional printing, thermoset molding, etc. Some embodiments comprise providing a base of the outer chamber, e.g., providing a polymer to provide a base of the outer chamber. Accordingly, some embodiments comprise polymerizing a composition, e.g., to provide a base of the outer chamber.

In some embodiments, the analyte is concentrated at the surface. at least 10-fold (e.g., at least 10, 15, 20, 25, 30, 35, 40, 45, 50, 55, 60, 65, 70, 75, 80, 85, 90, 90, 100, 150, 200, 250, 300, 350, 400, 450, 500, 550, 600, 650, 700, 750, 800, 850, 900, 950, or 100-fold or more) relative to its concentration prior to concentration by electrophoresis.

The electrophoretic cell technology finds use in the assay and detection technologies described herein. For example, the technology finds use for the concentration and/or surface-immobilization of nucleic acids (e.g., tumor-derived DNA or RNA) from a biological sample (e.g., plasma, urine, saliva, or other body fluids) for subsequent analysis by a sensitive and specific molecular analysis method. For example the technology finds use in the concentration and surface-immobilization of proteins (e.g., protein biomarkers) from a biological sample (e.g., plasma, urine, saliva, or other body fluids) for subsequent analysis by a sensitive and specific molecular analysis method. In particular, the technology finds use in concentrating analytes at a surface for analysis by single-molecule recognition through equilibrium Poisson sampling, SiMREPS, as described herein.

For example, in some embodiments, the methods comprise capturing the analyte from the inner chamber of an electrophoretic cell by contacting the electrophoretic cell to a surface and applying a voltage (e.g., 0-120 V (e.g., 5, 10, 15, 20, 30, 40, 50, 60, 70, 80, 90, 100, 110, or 120 V)) across the inner and outer chambers of the electrophoretic cell. For example, in some embodiments the technology comprises contacting the surface with the electrophoretic cell and applying a voltage of 0-120 V (e.g., 5, 10, 15, 20, 30, 40, 50, 60, 70, 80, 90, 100, 110, or 120 V) AC with a period of approximately 1 minute for a period of more than 1 minute (e.g., more than 1, 2, 3, 4, 5, 6, 7, 8, 9, 10, 20, 30, 40, 60, or 120 minutes). In some embodiments, methods comprise detecting the analyte at the solid-phase substrate. In some embodiments, detecting the analyte comprises use of a single-molecule detection method. In some embodiments, detecting the analyte comprises analyzing the kinetics of binding and dissociation of a detectably labeled query probe to the analyte with single-molecule sensitivity (see, e.g., U.S. patent application Ser. No. 14/589,467; Int'l Pat. App. No. PCT/US2015/044650; Int'l Pat. App. No. PCT/US2017/016977; and U.S. Provisional App. Ser. No. 62/468,578; each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In some embodiments, the technology provides methods for the analysis of a nucleic acid. In some embodiments, the technology provides methods for the analysis of a nucleic acid comprising fewer than 200 base pairs or nucleotides (e.g., comprising fewer than 200, 190, 180, 170, 160, 150, 140, 130, 120, 110, 100, 95, 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, or fewer than 10 nt or bp). In some embodiments, the technology provides methods for analyzing nucleic acids in or from a sample (e.g., a biological sample (e.g., a biofluid)). In some embodiments, the technology provides methods for analyzing nucleic acids derived from a biofluid, cell lysate, lysate of a single cell, lysate of a single subcellular compartment. For example, in some embodiments methods comprise adding a sample (e.g., a biological sample (e.g., a biofluid)) comprising a nucleic acid to an electrophoretic cell (e.g., to the inner chamber of an electrophoretic cell) and applying a voltage across the inner chamber and outer chamber of the electrolytic cell as described herein. In some embodiments, methods comprise adding a buffer to the outer chamber and providing a porous base (e.g., a polymer) to the outer chamber. Furthermore, in some embodiments the methods comprise capturing the nucleic acid at the surface of a solid-phase substrate.

In some embodiments, the technology related to use of the electrophoretic cell comprises a denaturing step. For example, in some embodiments of the methods, a sample is heated to a temperature that denatures a biomolecule analyte in the sample. In some embodiments, the denaturing step occurs prior to applying a voltage between the inner and outer chambers. In some embodiments, the denaturing step occurs after applying a voltage across the inner and outer chambers. In some embodiments, the denaturing step comprises heating the sample (e.g., heating to a temperature of at least 40, 45, 50, 55, 60, 65, 70, 75, 80, 85, 90, 95, 100, 105, or 110° C.). In some embodiments, the sample is treated with a chemical denaturant.

In some embodiments, denaturing (e.g., thermal denaturing) occurs in the presence of a carrier oligonucleotide or one or more oligonucleotides that prevent(s) formation of secondary structure or other hybridization of the target to other nucleic acids in the mixture. Accordingly, some embodiments comprise adding a carrier oligonucleotide or one or more oligonucleotides, e.g., to prevent(s) formation of secondary structure or other hybridization of the target to other nucleic acids in the mixture. In some embodiments, the carrier oligonucleotide or one or more oligonucleotides that prevent(s) formation of secondary structure or other hybridization of the target to other nucleic acids in the mixture is added to the sample; in some embodiments, the carrier oligonucleotide or one or more oligonucleotides that prevent(s) formation of secondary structure or other hybridization of the target to other nucleic acids in the mixture is added to the electrophoretic cell.

In some embodiments, an enzyme (e.g., a protease (e.g., proteinase K, peptide hydrolase), a lipase) is added prior to denaturing (e.g., prior to thermal denaturing). In some embodiments, the enzyme is added to the sample; in some embodiments, the enzyme is added to the electrophoretic cell; in some embodiments, the enzyme is added to a composition comprising the sample. In some embodiments, the enzyme has a charge that is opposite the charge of the analyte.

In some embodiments, a detergent is added prior to denaturing (e.g., a detergent including but not limited to SDS (e.g., 0-5%), Triton X-100, Triton X-114, deoxycholate, Brij-35, Brij-58, Tween 20, Tween 80, octyl glucoside, octyl thioglucoside, CHAPS, CHAPSO, etc.) In some embodiments, the detergent is added to the sample; in some embodiments, the detergent is added to the electrophoretic cell; in some embodiments, the detergent is added to a composition comprising the sample. In some embodiments, the detergent is nonionic or not charged (e.g., a chargeless detergent). In some embodiments, the detergent has a charge that is opposite the charge of the analyte.

In some embodiments, the sample comprises cells (e.g., isolated single cells) or subcellular components (e.g., isolated subcellular components), e.g., mitochondria, nuclei, exosomes, extracellular vesicles, etc. In some embodiments, cells or subcellular components are isolated by a microfluidic technique, a liquid-in-liquid emulsion technique, a micropipet manipulation technique, or other technology known in the art.

### Systems

Embodiments of the technology relate to systems for detecting analytes. For example, in some embodiments, the technology provides a system for quantifying one or more target analytes, wherein the system comprises a surface-bound capture probe or a surface-bound moiety that stably binds the target analyte. In some embodiments, the surface-bound capture probe or the surface-bound moiety stably binds the analyte via a binding site, a epitope, or a recognition site (e.g., a first binding site, a first epitope, or a first recognition site). In some embodiments, systems further comprise a query probe that binds the target analyte with a low affinity at a second binding site, a second epitope, or a second recognition site. In some embodiments, the query probe is freely diffusible in the bulk solution contacting the surface of the system. Furthermore, some system embodiments comprise a detection component that records a signal from the interaction of the query probe with the target analyte. For example, in some embodiments the detection component records the change in the signal as a function of time produced from the interaction of the query probe with the target analyte. In some embodiments, the detection component records the spatial position (e.g., as an x, y coordinate pair) and intensity of binding and dissociation events of the query probe to and from said target analyte. In some embodiments, the detection component records the spatial position (e.g., as an x, y coordinate pair) and the beginning and/or ending time of binding and dissociation events of the query probe to and from said target analyte. In some embodiments, the detection component records the spatial position (e.g., as an x, y coordinate pair) and the length of time of binding and dissociation events of the query probe to and from said target analyte.

Embodiments of systems further comprise an ATPS or electrophoretic cell as described herein, e.g., to concentrate an analyte for delivery to the surface of the system (e.g., a surface comprising a surface-bound capture probe or a surface-bound moiety that stably binds the target analyte).

For example, in some embodiments, system embodiments comprise a composition comprising two or more aqueous solutions that form an ATPS, e.g., when the two or more aqueous solutions are mixed. In some embodiments, systems comprise PEG and a citrate buffer (e.g., citric acid and a citrate salt). In some embodiments, systems comprise PEG, a citrate buffer (e.g., citric acid and a citrate salt), and a second salt (e.g., sodium chloride). In some embodiments, systems comprise a composition comprising PEG having a mass fraction in the composition that is greater than 30% (e.g., greater than 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 70, 71, 72, 73, 74, or 75%). In some embodiments, systems comprise a composition comprising citrate having a mass fraction in the composition of less than 2.5% (e.g., less than 1.00, 1.25, 1.50, 1.75, 2.00, 2.25, or 2.50%). In some embodiments, systems comprise a composition comprising sodium chloride at a concentration of more than 0.25 M (e.g., more than 0.25, 0.3, 0.35, 0.4, 0.45, 0.5, 0.55, 0.6, 0.65, 0.7, 0.75, 0.8, 0.85, 0.9, 1.0, 1.1, 1.2, 1.3, 1.4, or more than 1.5 M). In some embodiments, systems comprise PEG having mean molecular weight in the composition of between 1000 and 6000 Da (e.g., 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 Da). In some embodiments, systems comprise citric acid and sodium citrate in a composition, wherein the ratio of citric acid to sodium citrate in the composition provides a pH between 5.5 and 8.5 (e.g., a pH of 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5).

Some system embodiments comprise an electrophoretic cell as described herein. For example, in some embodiments a system comprises an electrophoretic cell (10) comprising an inner chamber (11) and an outer chamber (12) (see, e.g., FIG. 9). The inner chamber is capable of holding a solution comprising an analyte (13). The outer chamber is capable of holding a buffer (16) (e.g., a solution comprising an electrolyte (e.g., ions (e.g., cations, anions))). The inner chamber (11) comprises a wall surrounding an internal volume. The wall physically and electrically separates the internal volume from the buffer in the outer chamber (12). Accordingly, in some system embodiments the wall of the electrophoretic cell is an insulator (e.g., a non-conductor). In some embodiments, the inner chamber (11) comprises an opening (15) that places the solution comprising the analyte in contact with a surface (14) of the system. In some embodiments, the opening (15) of the inner chamber (11) has a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm). Accordingly, in some embodiments the composition comprising the analyte (13) contacts the surface (14) of the system over an area having a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm; e.g., over an area of less than approximately 100 mm², e.g., less than 100, 95, 90, 85, 80, 75, 70, 65, 60, 55, 50, 45, 40, 35, 30, 25, 20, 18, 16, 14, 12, 10, 9, 8, 7, 6, 5, 4, 3, 2, 1, 0.8, 0.7, 0.6, 0.5, 0.4, 0.3, 0.2, 0.1, 0.08, 0.06, 0.05, 0.02, or 0.01 mm²).

In some embodiments, the outer chamber (12) comprises a base (17). In some embodiments, the base (17) comprises a porous material (e.g., nanoporous, microporous, mesoporous) that allows passage of electrolytes between the inner chamber (11) and outer chamber (12) and minimizes and/or eliminates flow of analyte between the inner chamber (11) and outer chamber (12). In some embodiments, the base (17) comprises a porous material that is a polymer. In some embodiments, the base (17) comprises a porous material that is a polyacrylamide. Optionally, in some embodiments, the pores of the base (17) are blocked with a suitable material (e.g., a branched polymer (e.g., dextran sulfate)) to minimize or eliminate the migration of the analyte into the outer chamber base (17). The technology is not limited to any particular material for the base (17) provided it is a porous material comprising pore sizes that are small enough to exclude the analyte while being large and contiguous enough to permit flow of current (e.g., electrolytes) between the inner chamber (11) and the outer chamber (12). Accordingly, some system embodiments comprise a porous material that finds use in providing a base for the outer chamber. In some embodiments, systems comprise a composition (e.g., a composition comprising monomers) that is used to produce a porous material (e.g., a polymer) to provide a base for the outer chamber.

In some embodiments, the wall of the inner chamber (11) does not contact the surface (14) of the system. Accordingly, in some embodiments, the electrophoretic cell (10) comprises a gap (18) between the wall of the inner chamber (11) and the surface (14). In some embodiments, the wall of the inner chamber (11) is separated from the surface (14) by a distance of approximately 10 to 100 μm (e.g., 10, 15, 20, 25, 30, 35, 40, 45, 50, 55, 60, 65, 70, 75, 80, 85, 90, 95, or 100 μm) to provide a gap (18) between the wall of the inner chamber (11) and the surface (14) (see, e.g., FIG. 9). In some embodiments, the gap (18) allows current to flow between the inner chamber (11) and the outer chamber (12) (see, e.g., FIG. 9). In some embodiments, the gap (18) comprises the same material as the base (17).

In some embodiments, the electrophoretic cell (10) comprises an anode and a cathode. In some embodiments, the anode and cathode provide a voltage (e.g., a potential) (19) across the inner chamber (11) and the outer chamber (12). In some embodiments, the anode is electrically connected to the inner chamber (11) and the cathode is electrically connected to the outer chamber (12) (e.g., to move a negatively charged analyte to the surface (14)). In some embodiments, the cathode is electrically connected to the inner chamber (11) and the anode is electrically connected to the outer chamber (12) (e.g., to move a positively charged analyte to the surface (14)).

Some system embodiments further comprise a voltage supply. In some embodiments, the voltage supply is a source of direct current and in some embodiments the voltage supply is a source of alternating current. In some embodiments, the voltage supply provides a voltage of between 0-120 V (e.g., 0, 5, 10, 20, 25, 30, 35, 40, 45, 50, 55, 60, 65, 70, 75, 80, 85, 90, 95, 100, 105, 110, 115, 120 V). In some embodiments, the voltage supply provides a voltage that varies with a period of approximately 1 minute.

System embodiments comprise analytical processes (e.g., embodied in a set of instructions, e.g., encoded in software, that direct a microprocessor to perform the analytical processes) to identify an individual molecule of the target analyte. In some embodiments, analytical processes use the spatial position data and timing (e.g., start, end, or length of time) of repeated binding and dissociation events to said target analyte as input data.

Embodiments of systems are not limited in the analyte that is detected. For example, in some embodiments the analyte is polypeptide, e.g., a protein or a peptide. In some embodiments, the target analyte is a nucleic acid. In some embodiments, the target analyte is a small molecule.

In some embodiments, the interaction between the target analyte and the query probe is distinguishably influenced by a covalent modification of the target analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer.

In some embodiments, the query probe is a low-affinity antibody, antibody fragment, or nanobody.

In some embodiments, the query probe is a DNA-binding protein, RNA-binding protein, or a DNA-binding ribonucleoprotein complex.

In some embodiments, the analyte is a nucleic acid comprising a covalent modification to a nucleobase, a ribose, or a deoxyribose moiety of the target analyte.

In some embodiments, the capture probe is a high-affinity antibody, antibody fragment, or nanobody. In some embodiments, the capture probe is a nucleic acid. In some embodiments, capture is mediated by a covalent bond cross-linking the target analyte to the surface. In some embodiments, the target analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

Some system embodiments of the technology comprise components for the detection and quantification of a target analyte. Systems according to the technology comprise, e.g., a solid support (e.g., a microscope slide, a coverslip, an avidin (e.g., streptavidin)-conjugated microscope slide or coverslip, a solid support comprising a zero mode waveguide array, or the like), and a query probe as described herein.

Some system embodiments comprise a detection component that is a fluorescence microscope comprising an illumination configuration to excite bound query probes (e.g., a prism-type total internal reflection fluorescence (TIRF) microscope, an objective-type TIRF microscope, a near-TIRF or HiLo microscope, a confocal laser scanning microscope, a zero-mode waveguide, and/or an illumination configuration capable of parallel monitoring of a large area of the slide or coverslip (>100 μm²) while restricting illumination to a small region of space near the surface). Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some particular embodiments comprise a component configured for lens-free imaging, e.g., a lens-free microscope, e.g., a detection and/or imaging component for directly imaging on a detector (e.g., a CMOS) without using a lens.

Some embodiments comprise a computer and software encoding instructions for the computer to perform, e.g., to control data acquisition and/or analytical processes for processing data.

Some embodiments comprise optics, such as lenses, mirrors, dichroic mirrors, optical filters, etc., e.g., to detect fluorescence selectively within a specific range of wavelengths or multiple ranges of wavelengths.

For example, in some embodiments, computer-based analysis software is used to translate the raw data generated by the detection assay (e.g., the presence, absence, or amount of one or more analytes, e.g., as a function time and/or position (e.g., x, y coordinates) on the surface) into data of predictive value for a clinician. The clinician can access the predictive data using any suitable means.

Some system embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

The term “computer-readable medium” as used herein refers to any media that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., presence, absence, concentration of an analyte). For example, some embodiments contemplate a system that comprises a processor, a memory, and/or a database for, e.g., storing and executing instructions, analyzing fluorescence, image data, performing calculations using the data, transforming the data, and storing the data. It some embodiments, an algorithm applies a statistical model (e.g., a Poisson model or hidden Markov model) to the data.

Many diagnostics involve determining the presence of, or a nucleotide sequence of, one or more nucleic acids.

In some embodiments, an equation comprising variables representing the presence, absence, concentration, amount, or sequence properties of one or more analytes produces a value that finds use in making a diagnosis or assessing the presence or qualities of an analyte. As such, in some embodiments this value is presented by a device, e.g., by an indicator related to the result (e.g., an LED, an icon on a display, a sound, or the like). In some embodiments, a device stores the value, transmits the value, or uses the value for additional calculations. In some embodiments, an equation comprises variables representing the presence, absence, concentration, amount, or properties of one or more analytes.

Thus, in some embodiments, the present technology provides the further benefit that a clinician, who is not likely to be trained in analytical assays, need not understand the raw data. The data are presented directly to the clinician in its most useful form. The clinician is then able to utilize the information to optimize the care of a subject. The present technology contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed and a profile is produced that is specific for the diagnostic or prognostic information desired for the subject. The profile data are then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may chose further intervention or counseling based on the results. In some embodiments, the data are used for research use. For example, the data may be used to further optimize the inclusion or elimination of markers as useful indicators of a particular condition associated with the disease.

The technology provides a super-resolution identification, detection, quantification, and/or characterization of analytes based on transformation of signals produced by transient binding of query probes to analytes into data providing information relating to the identification, detection, quantification, and/or characterization of analytes. Some embodiments comprise one or more steps as described herein, e.g., one or more ordered steps as described herein. In some embodiments, one or more steps depend on and follow one or more other steps; however, some embodiments comprise one or more of the described steps without respect to any particular order.

As used herein, the term “dataset” or “movie” relates to data comprising a time series of sensor array data (e.g., from a CCD, intensified CCD, electron multiplying CCD, CMOS sensor, or the like), wherein each time point of the time series of sensor array data (or “frame”) comprises a set of signal intensity values as a function of position (e.g., x, y coordinates) within the sensor array. In some embodiments, the (x, y) position refers to the coordinates of sensor elements (e.g., pixels) in the horizontal (x) and vertical (y) directions within the two-dimensional sensor array, e.g., in a frame of a movie dataset.

In some embodiments, a dataset as described is collected from a sample comprising query probes (e.g., some embodiments comprise collecting a dataset from a sample comprising query probes). In some embodiments, the sample comprises analyte. In some embodiments, the sample is believed to comprise analyte. In some embodiments, it is not known if the sample comprises analyte.

Some embodiments comprise generating a differential intensity map movie. For example, some embodiments comprise subtracting the intensity value of each pixel P in each frame N of the movie from the corresponding intensity value of the same pixel P in the next frame N+1 of the movie. The result of these subtractions is a time series of differential intensity maps comprising one fewer frame than the original movie. Some embodiments comprise subtracting the intensity value of each pixel P in each frame N of the movie from the corresponding intensity value of the same pixel P in frame N+2, N+3, N+4, N+n of the movie to produce a time series of differential intensity maps comprising 2, 3, 4, or n fewer frames than the original movie.

Some embodiments comprise recording one or more of the position, intensity, and/or frame number of each intensity maximum (e.g., corresponding to query probe binding events) within each frame of the differential intensity map. In some embodiments, the position is determined by a transformation of the data comprising, e.g., a two-dimensional Gaussian fitting, a centroid fitting, or other methods that are used to determine the position of a particle, e.g., in some embodiments with an error of 1 pixel or less.

Some embodiments comprise recording one or more of the position, intensity (e.g., absolute value of the intensity), and/or frame number of each intensity minimum (e.g., corresponding to query probe dissociation events) within each frame of the differential intensity map. In some embodiments, the position is determined by a transformation of the data comprising, e.g., a two-dimensional Gaussian fitting, a centroid fitting, or other methods that are used to determine the position of a particle, e.g., in some embodiments with an error of 1 pixel or less.

Some embodiments comprise combining (x, y) positions of intensity maxima and/or intensity minima Some embodiments further comprise performing clustering analysis (e.g., hierarchical clustering) on the (x, y) positions of intensity maxima and/or intensity minima to identify regions of high density of query probe binding and dissociation events. In some embodiments, the clustering analysis produces clusters wherein each cluster contains 1 or more binding and/or dissociation event(s) that are detected within a limited region of the sensor.

Some embodiments comprise calculating one or more statistical measures for the events within each cluster, including but not limited to, the number of query probe binding and/or dissociation events; one or more of the mean, median, maximum, minimum, range, and standard deviation of the number of frames between a given binding event and the next dissociation event; one or more of the mean, median, maximum, minimum, range, and standard deviation of the number of frames between a given dissociation event and the next binding event; one or more of the mean, median, maximum, minimum, range, and standard deviation of the (x, y) position of query probe binding and dissociation events; and/or one or more of the mean, median, maximum, minimum, range, and standard deviation of the signal intensity change associated with query probe binding and dissociation events.

Some embodiments comprise comparing the statistics measured as described above for each cluster of query probe binding events to statistics measured using a standard reference material (e.g., a positive control). Some embodiments comprise comparing the statistics measured as described above for each cluster of query probe binding events to statistics measured using a negative control (e.g., a comprising no analyte, a substance closely related to the analyte, an analyte comprising a modification and/or not comprising a modification, etc.). In some embodiments, comparing the statistics measured as described above for each cluster of query probe binding events to statistics measured using a standard reference material and/or a negative control is used to determine whether the cluster of query probe binding events is probable to have originated from query probe binding to a single molecule of the target analyte.

Some embodiments comprise calculating the number of clusters in the dataset that represent query probe binding to the target analyte. In some embodiments, calculating the number of clusters in the dataset that represent query probe binding to the target analyte comprises using one or more of the statistical tests described above. In some embodiments, calculating the number of clusters in the dataset that represent query probe binding to the target analyte provides a measure of the number of analytes (e.g., the apparent number of analytes) present in the region of the imaging surface that was assayed by the method. In some embodiments, calculating the number of clusters in the dataset that represent query probe binding to the target analyte provides a measure of the concentration of analyte, provides an indication that the analyte is present or absent in the sample, and/or provides an indication of the state (e.g., modified, not modified) of the analyte in the sample.

Some embodiments optionally comprise comparing the apparent number, concentration, state, presence, or absence of analyte as described above to a previously determined value of apparent number, concentration, state, presence, or absence of analyte for a known analyte concentration. Some embodiments comprise use of a standard curve (e.g., generated with one or more compositions comprising a standard reference material of the target analyte having known concentrations) to determine the concentration of the target analyte in the sample.

In some embodiments, steps of the described methods are implemented in software code, e.g., a series of procedural steps instructing a computer and/or a microprocessor to produce and/or transform data as described above. In some embodiments, software instructions are encoded in a programming language such as, e.g., BASIC, C, C++, Java, MATLAB, Mathematica, Perl, Python, or R.

In some embodiments, one or more steps or components of the super-resolution identification, detection, quantification, and/or characterization of analytes are provided in individual software objects connected in a modular system. In some embodiments, the software objects are extensible and portable. In some embodiments, the objects comprise data structures and operations that transform the object data. In some embodiments, the objects are used by manipulating their data and invoking their methods. Accordingly, embodiments provide software objects that imitate, model, or provide concrete entities, e.g., for numbers, shapes, data structures, that are manipulable. In some embodiments, software objects are operational in a computer or in a microprocessor. In some embodiments, software objects are stored on a computer readable medium.

In some embodiments, a step of a method described herein is provided as an object method. In some embodiments, data and/or a data structure described herein is provided as an object data structure.

### Analytes

The technology is not limited in the analyte that is detected, quantified, identified, or otherwise characterized (e.g., presence, absence, amount, concentration, state). The term “analyte” as used herein is a broad term and is used in its ordinary sense, including, without limitation, to refer to a substance or chemical constituent in a sample such as a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte comprises a salt, sugars, protein, fat, vitamin, or hormone. In some embodiments, the analyte is naturally present in a biological sample (e.g., is “endogenous”); for example, in some embodiments, the analyte is a metabolic product, a hormone, an antigen, an antibody, and the like. Alternatively, in some embodiments, the analyte is introduced into a biological organism (e.g., is “exogenous), for example, a drug, drug metabolite, a drug precursor (e.g., prodrug), a contrast agent for imaging, a radioisotope, a chemical agent, etc. The metabolic products of drugs and pharmaceutical compositions are also contemplated analytes.

In some embodiments (e.g., embodiments related to an electrophoretic cell), the analyte is charged. For example, in some embodiments the analyte carries a negative charge (e.g., a net negative charge). In some embodiments, the analyte carries a positive charge (e.g., a net positive charge). In some embodiments, the analyte carries a partial charge (e.g., the analyte carries a partial negative or partial positive charge). In some embodiments, the charge state of the analyte is unknown. In some embodiments, the analyte responds to an electric potential (e.g., a voltage) by moving toward an anode or a cathode. In some embodiments, the analyte is derivatized (e.g., covalently, non-covalently) with a moiety that produces a charged molecule (e.g., a negatively charged molecule, a positively charged molecule, a partially negatively charged molecule, a partially positively charged molecule). In some embodiments, the analyte binds to a moiety that produces a charged complex (e.g., a negatively charged complex, a positively charged complex, a partially negatively charged complex, a partially positively charged complex). Thus, in some embodiments a molecule (e.g., an analyte derivatized with a moiety) responds to an electric potential (e.g., a voltage) by moving toward an anode or a cathode. In some embodiments a complex (e.g., an analyte bound to a moiety) responds to an electric potential (e.g., a voltage) by moving toward an anode or a cathode.

In some embodiments, the analyte is a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a combination of one or more of a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc.

In some embodiments, the analyte is part of a multimolecular complex, e.g., a multiprotein complex, a nucleic acid/protein complex, a molecular machine, an organelle (e.g., a cell-free mitochondrion, e.g., in plasma; a plastid; golgi, endoplasmic reticulum, vacuole, peroxisome, lysosome, and/or nucleus), cell, virus particle, tissue, organism, or any macromolecular complex or structure or other entity that can be captured and is amenable to analysis by the technology described herein (e.g., a ribosome, spliceosome, vault, proteasome, DNA polymerase III holoenzyme, RNA polymerase II holoenzyme, symmetric viral capsids, GroEL/GroES; membrane protein complexes: photosystem I, ATP synthase, nucleosome, centriole and microtubule-organizing center (MTOC), cytoskeleton, flagellum, nucleolus, stress granule, germ cell granule, or neuronal transport granule). For example, in some embodiments a multimolecular complex is isolated and the technology finds use in characterizing, identifying, quantifying, and/or detecting one or more molecules (analytes) associated with (e.g., that is a component of) the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

In some embodiments, the analyte is chemically modified to provide a site for query probe binding. For instance, in some embodiments, beta-elimination of phosphoserine and phosphothreonine under strongly basic conditions is used to introduce an alkene, followed by Michael addition of a nucleophile such as a dithiol to the alkene. The remaining free thiol is then used for conjugation to a maleimide-containing oligonucleotide with a sequence complementary to an oligonucleotide query probe. The post-translational modifications phosphoserine and phosphothreonine may then be probed using the query probe and analyzed as described herein.

As used herein “detect an analyte” or “detect a substance” will be understood to encompass direct detection of the analyte itself or indirect detection of the analyte by detecting its by-product(s).

### Samples

In some embodiments, analytes are isolated from a biological sample. Analytes can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof. Analytes can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), breath condensate, and/or other specimens.

Any tissue or body fluid specimen may be used as a source of analytes for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Analytes can also be isolated from cultured cells, such as a primary cell culture or a cell line. The cells or tissues from which analytes are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Analytes (e.g., nucleic acid molecules, polypeptides, lipids) can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281).

In some embodiments, the technology provides for the size selection of analytes, e.g., to provide a defined size range of molecules including the target analytes.

### Uses

Various embodiments relate to the detection of a wide range of analytes. For example, in some embodiments the technology finds use in detecting a nucleic acid (e.g., a DNA or RNA). In some embodiments, the technology finds use in detecting a nucleic acid comprising a particular target sequence. In some embodiments, the technology finds use in detecting a nucleic acid comprising a particular mutation (e.g., a single nucleotide polymorphism, an insertion, a deletion, a missense mutation, a nonsense mutation, a genetic rearrangement, a gene fusion, etc.). In some embodiments, the technology finds use in detection a polypeptide (e.g., a protein, a peptide). In some embodiments, the technology finds use in detecting a polypeptide encoded by a nucleic acid comprising a mutation (e.g., a polypeptide comprising a substitution, a truncated polypeptide, a mutant or variant polypeptide).

In some embodiments, the technology finds use in detecting post-translational modifications to polypeptides (e.g., phosphorylation, methylation, acetylation, glycosylation (e.g., O-linked glycosylation, N-linked glycosylation, ubiquitination, attachment of a functional group (e.g., myristoylation, palmitoylation, isoprenylation, prenylation, farnesylation, geranylation, geranylgeranylation, glypiation, glycosylphosphatidylinositol (GPI) anchor formation), hydroxylation, biotinylation, pegylation, oxidation, SUMOylation, disulfide bridge formation, disulfide bridge cleavage, proteolytic cleavage, amidation, sulfation, pyrrolidone carboxylic acid formation. In some embodiments, the technology finds use in the detection of the loss of these features, e.g., dephosporylation, demethylation, de acetylation, de glycosylation, deamidation, dehydroxylation, deubiquitination, etc. In some embodiments, the technology finds use in detecting epigenetic modifications to DNA or RNA (e.g., methylation (e.g., methylation of CpG sites), hydroxymethylation). In some embodiments, the technology finds use in detecting the loss of these features, e.g., demethylation of DNA or RNA, etc. In some embodiments, the technology finds use in detecting alterations in chromatin structure, nucleosome structure, histone modification, etc., and in detecting damage to nucleic acids.

In some embodiments, the technology finds use as a molecular diagnostic assay, e.g., to assay samples having small specimen volumes (e.g., a droplet of blood, e.g., for mail-in service). In some embodiments, the technology provides for the early detection of cancer or infectious disease using sensitive detection of very low-abundance analyte biomarkers. In some embodiments, the technology finds use in molecular diagnostics to assay epigenetic modifications of protein biomarkers (e.g., post-translational modifications).

In some embodiments, the technology finds use in characterizing multimolecular complexes (e.g., characterizing one or more components of a multimolecular complex), e.g., a multiprotein complex, a nucleic acid/protein complex, a molecular machine, an organelle (e.g., a cell-free mitochondrion, e.g., in plasma), cell, virus particle, organism, tissue, or any macromolecular structure or entity that can be captured and is amenable to analysis by the technology described herein. For example, in some embodiments a multimolecular complex is isolated and the technology finds use in characterizing, identifying, quantifying, and/or detecting one or more molecules (analytes) associated with the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

In some embodiments, single cells (e.g., human, mammalian, vertebrate, eukaryotic, or prokaryotic cells), single organelles (e.g., mitochondria, nuclei, Golgi bodies), subcellular vesicles (e.g., exosomes), and/or subcellular cohesive particles (e.g., membraneless organelles (e.g., intracellular exosome, protein aggregates)) are isolated (e.g., in separate wells, microwells, microfluidic channels, sample chambers, test tubes, microcentrifuge tubes, or other vessel or compartment, or in separate regions of the same compartment). In some embodiments, single cells, organelles, subcellular vesicles, and/or subcellular cohesive particles are isolated by physical extraction (e.g., using suction, fluidic force, or optical trapping) from living or chemically fixed cells, tissues, colonies, or biofluids. In some embodiments, the single cells, organelles, subcellular vesicles, and/or subcellular cohesive particles are isolated from formalin-fixed, paraffin-embedded (FFPE) tissue. In some embodiments, single cells, organelles, subcellular vesicles, and/or subcellular cohesive particles are isolated from suspension by fluidic (e.g., by fluorescence-activated cell sorting, FACS) or microfluidic manipulation, or by segregation into separate wells or regions of a culture dish, microscope slide, or coverslip, or in different regions of a 3D cell culture medium or matrix. In some embodiments, an electric and/or magnetic field is used to effect the separation of a single cell, organelle, subcellular vesicle, and/or subcellular cohesive particle from other biological components. That is, in some embodiments, an electric and/or magnetic field is used to isolate single cells, organelles, subcellular vesicles, and/or subcellular cohesive particles.

In some embodiments, single cells (e.g., human, mammalian, vertebrate, eukaryotic, or prokaryotic cells), single organelles (e.g., mitochondria, nuclei, Golgi bodies), subcellular vesicles (e.g., exosomes), and/or subcellular cohesive particles (e.g., membraneless organelles (e.g., intracellular exosome, protein aggregates)) are treated (e.g., with cell lysis buffers; detergents such as SDS, Triton X-100, or deoxycholate; and/or enzymes such as proteases, lipases, or nucleases) to release an analyte from each single cell, organelle, subcellular vesicle, and/or subcellular cohesive particle. In some embodiments, single cells, organelles, subcellular vesicles, and/or subcellular cohesive particles are treated in situ on or near a solid support to liberate the analyte for capture on a nearby region of the solid support.

In some embodiments, the analyte is captured at the surface of a solid support. In some embodiments, the analyte is concentrated by mixing the contents of the single cell, organelle, subcellular vesicle, and/or subcellular cohesive particle with another mixture to form an aqueous two-phase system prior to capture on the solid support.

In some embodiments, the analyte is detected by a single molecule detection method (e.g., SiMREPS).

Although the disclosure herein refers to certain illustrated embodiments, it is to be understood that these embodiments are presented by way of example and not by way of limitation.

### EXAMPLES

### Methods

All unmodified DNA oligonucleotides used in this study were purchased from Integrated DNA Technologies (IDT) with standard desalting purification. Fluorescent probe oligonucleotides were purchased from IDT with either a 5′ or 3′ fluorophore modification and high-performance liquid chromatography (HPLC) purification. Biotinylated LNA capture probes were purchased from Exiqon with HPLC purification. Double-stranded DNA substrates were prepared by combining complementary oligonucleotides (20 μM each strand) in annealing buffer (10 mM Tris-HCl [pH 8.0 at 25° C.], 50 mM NaCl, 1 mM EDTA), heating at 95° C. for 5 min, followed by slow cooling at room temperature for 15 min.

SiMREPS experiments were performed on an Olympus IX-81 objective-type TIRF microscope equipped with a 60× oil-immersion objective (APON 60XOTIRF, 1.49 NA) with both Cell̂TIRF and z-drift control modules, and an EMCCD camera (IXon 897, Andor, EM gain 150). In some experiments, sample cells were constructed as previously described (see, e.g., Johnson-Buck et al. (2015) “Kinetic fingerprinting to identify and count single nucleic acids” Nat. Biotechnol. 33: 730-32, incorporated herein by reference). Briefly, glass coverslips were passivated with a 1:10 mixture of biotin-PEG-5000 and mPEG-5000 (Laysan Bio, Inc.), and stored for up to two weeks in the dark under nitrogen. Directly prior to single molecule experiments, 20 μL pipette tips (Eppendorf, low adhesion) were cut to a 1 cm total length, and the non-cut base was adhered to the coated glass coverslip via epoxy (Double Bubble, Hardman Adhesives) to form the completed water-tight sample cell. Where indicated, a sample well comprising a restricted opening and an ATPS concentration were used to deposit analyte onto sample cells.

The SiMREPS protocol used in this study was based on the previously described method (see, e.g., Johnson-Buck et al. (2015) “Kinetic fingerprinting to identify and count single nucleic acids” Nat. Biotechnol. 33: 730-32, incorporated herein by reference), with three main modifications: (1) the LNA concentration was increased from 20 nM to 100 nM during surface coating, (2) a thermal denaturing step, efficacious for the low- and sub-nanomolar DNA concentrations used here, was added directly before analyte capture to increase access to the target DNA strand, and (3) two additional oligonucleotides including an LNA blocker and wild-type competitor (WTC) were added to the imaging solution when probing T790M samples. All DNA handling was performed in GeneMate low-adhesion 1.7-mL micro centrifuge tubes, and all dilutions and denaturing steps were performed in the presence of 2 μM poly-T oligodeoxyribonucleotide (dT10). The updated protocol was performed as follows. First, the sample cell was washed with T50 buffer (10 mM Tris-HCl [pH 8.0 at 25° C.], 1 mM EDTA), and then incubated with 40 μL of 1 mg/mL streptavidin for 10 min. The sample cell was then washed with T50 buffer to remove excess streptavidin, incubated with 40 μL of 100 nM biotinlyated LNA for 10 min, and washed with 1×PBS. Target DNA oligonucleotide was then denatured at 95° C. for 3 min (unless otherwise specified) in a thermocycler, cooled in room temperature water for 5 min (via partial submersion), and then immediately added to the LNA coated surface. DNA capture was performed for 1 hour at room temperature in a humidified chamber. After capture, excess DNA was removed with 4×PBS. Directly before imaging, imaging buffer containing 25 nM fluorescent probe, 4×PBS, 5 mM 3,4-dihydroxybenzoate, 50 nM protocatechuate dioxygenase, and 1 mM Trolox was added to the sample cell. When probing T790M specifically, 25 nM LNA blocker and 1 μM WTC were also added to the imaging solution). Transient binding of the fluorescent probe was monitored for 10 min under TIRF illumination by 640 nM laser light with a 500 ms exposure time (acquiring 1200 total frames).

Two different analytical pipelines were used to investigate kinetic data in this study. The first analytic pipeline termed “diffraction limited” employs custom Matlab code to identify sites of fluorescent probe binding and dissociation and calculate intensity-versus-time trajectories (see, e.g., Johnson-Buck et al. (2015) “Kinetic fingerprinting to identify and count single nucleic acids” Nat. Biotechnol. 33: 730-32, incorporated herein by reference). Hidden Markov modeling (HMM) is then applied using the QuB software suite (State University of New York at Buffalo) to determine the number of binding and dissociation events (N_{b+d}) and the median fluorescent probe bound (τ_{bound}) and unbound (τ_{unbound}) time for each candidate molecule (Blanco & Walter (2010) “Analysis of complex single-molecule FRET time trajectories” Methods Enzymol. 472: 153-78, incorporated herein by reference). Based on (i) no DNA, (ii) wild-type DNA only, and (iii) mutant DNA only control experiments, kinetic thresholds were defined to achieve nominal false positive frequency while retaining high sensitivity for true mutant candidates. Thresholds applied for all DNA targets include a signal-to-noise ratio >3, mean bound state intensity >500 counts above the mean background intensity, maximum τ_{bound} median ≤20 s, maximum τ_{unbound} median ≤30 s, and N_{b+d}≥20. For Exon 19 deletion quantification, the minimum τ_{bound} and τ_{unbound} median threshold was set to ≥2.5 s, while for T790M quantification the minimum Mound and τ_{unbound} median threshold was set to ≥3 s.

Double-stranded DNA substrates were prepared by annealing chemically synthesized oligonucleotides (“Synthetic” substrates, see above), or by restriction enzyme digestion of plasmid DNA (“Cloned” substrates). The cloning strategy used was such that the resulting Cloned DNA substrates were identical in sequence to the Synthetic substrates. All of the plasmids used in this study are available through Addgene (www.addgene.org).

Plasmids containing 4 copies of the desired wild-type or mutant fragment were prepared using standard molecular biology techniques using enzymes purchased from New England Biolabs. Briefly, partially complementary forward and reverse oligonucleotides were purchased from IDT and phosphorylated using T4 polynucleotide kinase (#M0201S) according to the manufacturer's recommendations. Phosphorylated oligonucleotides were annealed by combining complementary strands at 1:1 stoichiometry and heating and slow cooling using a thermocycler. Annealed oligonucleotides were then filled in using either T4 DNA polymerase (#M0203S) or E. coli DNA Polymerase I (large Klenow fragment, #M0210S) to create fully-complementary, blunt-ended fragments, then ligated into pUC19 vector linearized with SmaI, and transformed into the JM109 strain of E. coli (Promega, P9751). Colonies were screened by PCR and clones containing ≥4 copies of the insert were selected. The plasmid sequences for all clones were confirmed by Sanger sequencing. Wild-type T790 and mutant T790M Cloned substrates were prepared from their respective multi-insert plasmids (pUC19_4 xT790_28 bp and pUC19_4 xT790M_28 bp) by digestion with MlyI and ZraI (#R0610S and #R0659S). The concentration of restriction digested fragments was determined by comparison with a standard curve after gel electrophoresis and SYBR gold staining on a 20% native polyacrylamide gel.

Statistical significance of the differences between mean counts per field of view was determined using a using 2-tailed, unpaired t test (GraphPad QuickCalcs, www.graphpad.com). Curve fitting was performed using OriginPro 8 software, and values were weighted using instrumental weighting by the respective error value during linear regression.

### Example 1

During the development of embodiments of the technology provided herein, experiments were conducted to concentrate a small nucleic acid at a surface for analysis. The experiment was conducted according to the method described herein and depicted in FIG. 1a. In brief, a DNA analyte is concentrated in the minority phase of an aqueous two-phase system and the analyte is subsequently captured from the minority phase at a surface for analysis (e.g., by single-molecule analysis (e.g., by SiMREPS as described herein)). In this method, centrifugation is typically carried out at approximately 1000×g for 10-120 minutes to provide high capture efficiency of the analyte at the coverslip surface. However, experiments conducted during the development of the technology provided herein indicated that centrifuging for approximately 1 minute at 1000×g is sufficient to provide phase separation.

Furthermore, additional data collected during these experiments indicated that an ATPS comprising a mixture of PEG 3350, sodium citrate, and sodium chloride effectively partitions and concentrates short single-stranded or double-stranded DNA (e.g., 10-100 nt or bp (e.g., 15-50 nt or bp (e.g., 20-30 nt or bp))). In a particular embodiment, the concentration of nucleic acid by the ATPS technology described herein increased the sensitivity of an assay for the epidermal growth factor receptor (EGFR) variant T790M by a factor of approximately 80 (see, e.g., FIGS. 1b and 1c), which resulted in a limit of detection of approximately 100 molecular copies. The technology thus has a sensitivity similar to the sensitivity of PCR analysis.

### Example 2

During the development of embodiments of the technology provided herein, custom sample wells were produced and tested that are designed for contacting the analyte-rich phase of the ATPS to the capture substrate. In particular, a custom sample well was produced that comprises a restricted opening (e.g., comprising an area of approximately 0.01-1 mm²) at the bottom of the well (see, e.g., FIG. 2). The sample well comprising the restricted opening provides an area of contact between the analyte-rich phase of the ATPS and the underlying substrate of approximately 0.01-1 mm². Accordingly, the sample well comprising the restricted opening focuses the analyte-rich phase over a small region (e.g., approximately 0.01-1 mm²) for efficient surface capture and, in some embodiments, detection of the analyte.

### Example 3

During the development of embodiment of the technology provided herein, experiments were conducted to detect analytes with high specificity and single-molecule sensitivity using single-molecule recognition through equilibrium Poisson sampling (SiMREPS), or single-molecule kinetic fingerprinting (FIG. 3a to FIG. 3e)

The detection method comprised heating the sample to denature double-stranded (duplex) DNA and convert it to single-stranded DNA. The brief heat-denaturation step was performed in the presence of high concentrations of single-stranded dT₁₀ carrier to inhibit re-annealing of complementary analyte strands. Next, single-stranded target DNA was captured by target gene-specific LNA capture probes immobilized at the slide surface. Remaining unbound DNA was washed away prior to analysis using a kinetic fingerprinting method as described herein. In these experiments, a mutant-specific fluorescent probe was used that is optimized to have fast binding and dissociation kinetics.

During the development of embodiments of the technology, experiments were conducted to test the impact of DNA strandedness and heat denaturation on the molecules detected (e.g., quantified) for two mutant target DNA alleles (FIGS. 3b to 3e). As shown in FIG. 3b, denaturing double-stranded DNA increased the detection of target DNA to a level similar to the detection of single-stranded DNA. Denaturing double-stranded DNA greatly increased the detection of target DNA relative to detection of non-denatured double-stranded DNA target in samples.

Also, during the development of embodiments of the technology described herein, experiments were conducted to record kinetic traces (e.g., using SiMREPS) using a mutant-specific fluorescent probe with mutant DNA (MUT DNA), with wild-type DNA (WT DNA), or without DNA (no DNA control) (FIGS. 3c and 3d). Probes for detecting EGFR Exon 19/deletion (c.2236_2250 del15) and EGFR Exon 20 T790/M (c.2369C>T) were used (FIGS. 3c and 3d, respectively). Kinetic trace data were used to construct standard curves from SiMREPS assays for EGFR Exon 19 deletion and EGFR Exon 20 T790M (FIG. 3e).

### Example 4

During the development of embodiments of the technology described herein, experiments were conducted to determine concentrations of PEG, citrate, and NaCl that produce a single aqueous phase or two aqueous phases. Cloud-point turbidity assay data were collected to produce a binodal curve (FIG. 4). The data indicate the combinations of PEG-3350 and sodium citrate dehydrate that produce a single aqueous phase or two aqueous phases in the presence of 2.8% w/w NaCl.

### Example 5

During the development of embodiments of the technology provided herein, experiments were conducted to test the effect of NaCl on the partitioning of analyte into the lower phase of a ATPS. Data were collected from experiments in which the partitioning of a Cy5-labeled 34-nucleotide single-stranded DNA molecule was measured in different aqueous systems. For each experiment, 100 μL of a 1-μM solution of the Cy5-oligonucleotide was tested in the following systems:
(1) water control
(2) 300 μL 60% PEG 3350+25 μL 33% sodium citrate
(3) 300 μL 60% PEG 3350+25 μL 33% sodium citrate+25 μL 5 M NaCl
(4) 300 μL 60% PEG 3350+25 μL 33% sodium citrate+50 μL 5 M NaCl
(5) 300 μL 60% PEG 3350+15 μL 33% sodium citrate+50 μL 5 M NaCl
(6) 300 μL 60% PEG 3350+25 μL 33% sodium citrate+100 μL 5 M NaCl.
The data collected indicated that mixtures (2) and (5) exhibited similar volumes of the lower citrate- and DNA-rich phase, but mixture (5) comprised a higher concentration of the DNA as indicated by a more intense blue color observed for mixture (5). Accordingly, the data indicated that addition of NaCl improves partitioning of the DNA oligonucleotide into the lower phase.

Furthermore, experiments were conducted in which a 1-μM solution of the Cy5-labeled 34-nucleotide DNA was added to a lyophilized mixture of sodium citrate, NaCl, and PEG 3350 to test the concentration of the DNA in the ATPS mixtures. Final concentrations of the components are indicated in Table 1 below:

TABLE 1

| | | | | Concentration |
|---|---|---|---|---|
| | Sodium | | | of DNA in |
| | Citrate | NaCl | PEG 3350 | lower phase |
| Mixture | (w/w) | (w/w) | (w/w) | (μM) |
| 1 | 1.20% | 2.80% | 39% | 61.0 +/− 4.8 |
| 2 | 1.50% | 2.80% | 39% | 23.9 +/− 1.3 |
| 3 | 2.00% | 2.80% | 39% | 13.1 +/− 0.4 |

The concentration of DNA in the lower phase was measured by UV-visible spectrophotometry. The data indicate that the ATPS provides an increasing concentration of DNA in the lower phase by decreasing the mass fraction of sodium citrate in the system while holding PEG 3350 and NaCl concentrations constant.

### Example 6

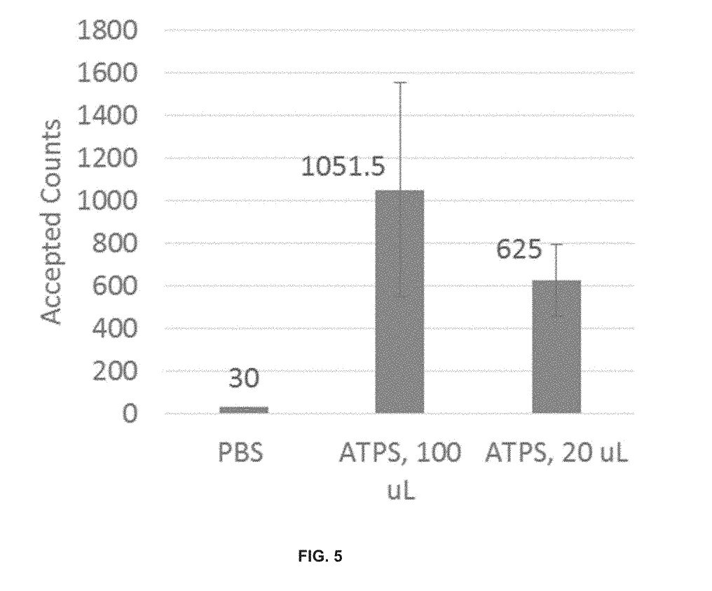

During the development of embodiments of the technology provided herein, experiments were conducted to test the detection of DNA by SiMREPS kinetic fingerprinting per field of view after a 1-hour incubation of the DNA in three different matrices: PBS (100 μL sample volume), PEG/sodium citrate/NaCl ATPS (100 μL sample volume); and PEG/sodium citrate/NaCl ATPS (20 μL sample volume). The indicated sample volumes were added to PBS or ATPS and the lower phases contacted to surfaces for immobilization and assay by SiMREPS. The data collected indicated that the technology provided a 5-fold reduction in the input volume of DNA while maintaining more than half the sensitivity improvement of the ATPS (FIG. 5).

### Example 7

During the development of embodiments of the technology provided herein, experiments were conducted to test PCR-free detection by SiMREPS with the aid of ATPS. In particular, data were collected from experiments in which endogenous low-molecular weight DNA fragments from the EGFR gene were isolated from a human urine sample and then analyzed by SiMREPS kinetic fingerprinting with the aid of an aqueous two-phase system comprising PEG 3350 (39% w/w), sodium citrate (1.2% w/w), and NaCl (2.8% w/w). DNA was denatured at 95° C. for 3 minutes in the presence of a carrier oligo(dT)₁₀ strand, then cooled to room temperature before mixing with a lyophilized solid mixture of PEG 3350, sodium citrate, and NaCl. While SiMREPS has a limit of detection of about 1 fM without ATPS, the use of ATPS here permits the detection of sub-femtomolar concentrations of DNA. The aqueous two-phase system yields sufficient sensitivity to detect wild-type DNA, but mutant DNA is not detected at levels above background (FIG. 6).

### Example 8

During the development of embodiments of the technology provided herein, experiments were conducted to test use a complementary oligonucleotide to prevent re-hybridization of double-stranded DNA following thermal denaturation and concentration in an aqueous two-phase system. In this set of experiments, a 28-base-pair double-stranded DNA fragment (corresponding to a region of the EGFR gene) was heat-denatured at 95° C. in 1×PBS in the presence or absence of a 14-base oligonucleotide (“Tile”) that is partly complementary to the target strand of the 28-base-pair duplex, but which does not obscure the remaining 14 base pairs (which would interfere with surface capture). The mixture was cooled following heat denaturation, then mixed thoroughly with lyophilized components to form an aqueous two-phase system (1.2% w/w sodium citrate, 2.8% w/w NaCl, 39% w/w PEG 3350). After concentration the DNA in the lower phase, the lower phase of the ATPS comprising concentrated DNA was contacted to a coverslip coated with an LNA-containing capture probe that is complementary to a portion of the target strand of the 28-base-pair duplex.

The data collected indicated that including the “Tile” oligonucleotide increased sensitivity in a SiMREPS kinetic fingerprinting assay by a factor of approximately 6 (as judged by the Accepted Counts, FIG. 7). The target DNA was present at a nominal concentration of 10 fM (10⁻¹⁴ M), and prepared at a volume of 10 μL prior to addition of ATPS-forming components. These concentrations corresponded to a total copy number of approximately 60,000 analyte molecules in the mixture. The imaging efficiency is thus approximately 6.7% per field of view (4000 counts/60,000 analyte molecules), and the overall capture efficiency of the analyte is estimated at 33% (since a single well is about 300 μm in diameter, comprising an area equivalent to about 5 fields of view, resulting in an estimated 20,000 molecules being immobilized over the entire imageable area of the coverslip).

Furthermore, longer double-stranded DNA targets are captured with higher efficiency when complementary short oligonucleotides (e.g., 14 base pairs in length), or “tiles,” are included to prevent or slow re-hybridization of the two complementary strands of the target. Data were collected from experiments in which tiles were used to improve capture of a 60 bp and 160 bp fragment of the EGFR gene containing the T790M mutation. Capture efficiency is increased approximately 8- to 9-fold by inclusion of tile oligos (FIG. 8).

### Example 9

During the development of embodiments of the technology described herein, experiments were conducted to test if ATPS can be used to concentrate RNA analytes. Data collected during these experiments indicated that RNA analytes are concentrated in the smaller-volume phase according to the technology described herein. In these experiments, two fluorescently labeled microRNAs approximately 22 nucleotides in length were used (e.g., miR-21 and miR-200a). The data collected indicated that the fluorophore-labeled miR-21 and miR-200a were sequestered in the bottom, citrate-rich phase of an aqueous two-phase system comprising PEG 3350 (16% w/w), sodium citrate (6% w/w), and NaCl (2.8% w/w).

### Example 10

During the development of embodiments of the technology provided herein, various concentration strategies were tested to increase the sensitivity of a TIRF-based SiMREPS assay for DNA. Several strategies involving pre-concentration, extended incubation, applied external voltage, or reduced capture area were attempted (Table 2). However, the only strategy yielding greater than a 10-fold higher sensitivity was an aqueous two-phase system containing PEG 3350, sodium citrate, and NaCl. In combination with this ATPS, restriction of the capture region using custom sample well geometries (see FIG. 2) provided a reproducible improvement in sensitivity greater than 50-fold.

TABLE 2

| | Relative |
|---|---|
| | sensitivity |
| Strategy | (counts/FOV) |
| Incubation in PBS buffer, 1 hour | 1 |
| Incubate in PBS buffer, 18 h | ~2 |
| Incubate in 2X SSC, pH 6.4, 10% dextran sulfate, 0.1% | ~1 |
| Tween-20, 20% formamide, 18 h | |
| Incubate in PBS + 10% dextran sulfate, 18 h | 3 |
| Restrict capture region to <1 mm² | ~3 |
| Parallel plate capacitor (applied external voltage) | ≤13 |
| Repeated mixing w/syringe pump, 1 h | 1 |
| Dynabead (~1 um) capture and magnetic deposition | <1 |
| Capture with streptavidin-coated silica beads (~100 nm) | <1 |
| and deposit by centrifugation | |
| Concentration through evaporation of solvent | ≤5 |
| Ethanol precipitation + resuspension in smaller volume | ~1 |
| ATPS with PEG/dextran | ~1 |
| ATPS with PEG 3350, sodium citrate, NaCl | >10 |
| ATPS with PEG 3350, sodium citrate, NaCl + restrict | >60 |
| capture region to <1 mm² | |

### Example 11

During the development of embodiments of the technology provided herein, experiments were conducted to test the effect of sample pH and citrate pH in the ATPS on the sensitivity of a single-molecule assay. The pH of a PBS buffer comprising a DNA analyte was varied and the effect was analyzed in a SiMREPS experiment. Additionally, the pH of citrate in a PEG/citrate/NaCl ATPS was varied and the effect measured.

When the pH of the sample was varied (pH of 8.74, 8.54, and 7.92), the accepted counts decreased very slightly as pH decreased from a standard pH of 8.04 PBS. The citrate of the ATPS dominates buffering capacity of the ATPS, thus the pH variance of PBS is negligible on the concentration of the analyte by ATPS as described herein.

To vary the pH of the ATPS components, the citrate/citric acid ratio or addition of HCl was used to lower the pH of the ATPS (pH of 7.58, 6.37, and 5.91 were tested). The data indicated that decreasing the ATPS citrate buffer pH decreased the accepted counts.

Thus, the data indicated that the pH of the DNA sample added to the ATPS does not appear to affect the SiMREPS counts significantly. The data indicated that lowering the pH of the ATPS system appears to decrease the counts.

### Example 12

During the development of embodiments of the technology provided herein, experiments were conducted to design and fabricate a sample cell for electrophoretic delivery and concentration of analytes at a surface 14 (see, e.g., FIG. 9). The sample cell 10 comprises an inner chamber 11 and an outer chamber 12. The inner chamber 11 is designed to hold a composition comprising an analyte (e.g., sample comprising analyte) 13, to provide contact of a composition comprising an analyte 13 with a surface, and to deposit an analyte on the surface. Accordingly, the inner chamber 11 comprises an opening 15 that contacts the surface 14 through which the composition comprising the analyte contacts the surface 14. In some embodiments, the inner chamber opening 15 has a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm). Accordingly, in some embodiments the composition comprising the analyte 13 contacts the surface 14 over an area having a width of less than approximately 10, 9, 8, 7, 6, 5, 4, 3, 2, or 1 mm (e.g., less than 10000, 9500, 9000, 8500, 8000, 7500, 7000, 6500, 6000, 5500, 5000, 4500, 4000, 3500, 3000, 2500, 2000, 1500, 1000, 950, 900, 850, 800, 750, 700, 650, 600, 550, 500, 450, 400, 350, 300, 250, 200, 150, or 100 μm; e.g., over an area of less than approximately 100 mm², e.g., less than 100, 95, 90, 85, 80, 75, 70, 65, 60, 55, 50, 45, 40, 35, 30, 25, 20, 18, 16, 14, 12, 10, 9, 8, 7, 6, 5, 4, 3, 2, 1, 0.8, 0.7, 0.6, 0.5, 0.4, 0.3, 0.2, 0.1, 0.08, 0.06, 0.05, 0.02, or 0.01 mm²).

The outer chamber 12 holds a buffer 16 (e.g., comprising small electrolytes (e.g., ions (e.g., cations, anions))) and surrounds the inner chamber 11. The outer chamber 12 comprises a base 17 comprising a nanoporous, mesoporous, or microporous material that permits the free flow of small electrolytes (e.g., buffer components (e.g., ions (e.g., cations, anions))) while preventing the migration of the analyte out of the inner chamber 11 (see, e.g., FIG. 9). In some embodiments, the nanoporous, mesoporous, or microporous material is a polymer (e.g., agarose, polyacrylamide, etc.)

In some embodiments, the inner chamber wall does not contact the surface 14. In some embodiments, the inner chamber wall is separated from the surface by a distance of approximately 10 to 100 μm (e.g., 10, 15, 20, 25, 30, 35, 40, 45, 50, 55, 60, 65, 70, 75, 80, 85, 90, 95, or 100 μm) to provide a gap 18 between the inner chamber 11 and the surface 14 (see, e.g., FIG. 9). In some embodiments, the gap 18 allows current to flow between the inner chamber 11 and the outer chamber 12 (see, e.g., FIG. 9).

In some embodiments, the surface 14 is a glass microscope coverslip.

In some embodiments, a voltage 19 is applied across the inner chamber 11 and outer chamber 12 to cause charged analytes (e.g., negatively charged DNA) to migrate towards the surface 14. While FIG. 9 shows a positive terminal placed at the outer chamber and a negative terminal placed at the inner chamber, the technology is not limited in this arrangement and includes embodiments in which a positive terminal placed at the inner chamber and a negative terminal placed at the outer chamber. In some embodiments, the outer chamber base 17 (e.g., comprising an agarose or polyacrylamide matrix) prevents flow of aqueous buffer solution between the inner chamber 11 and outer chamber 12 and minimizes or eliminates migration of the analyte out of the base of the inner chamber well. Optionally, in some embodiments the pores of base 17 (e.g., the agarose or polyacrylamide) are blocked with a suitable material (e.g., a branched polymer (e.g., dextran sulfate)) to minimize or eliminate the migration of the analyte into the outer chamber base 17. During the development of embodiments of the technology, fluorescently labeled DNA was used to confirm that dextran sulfate prevented migration of the DNA out of the inner chamber.

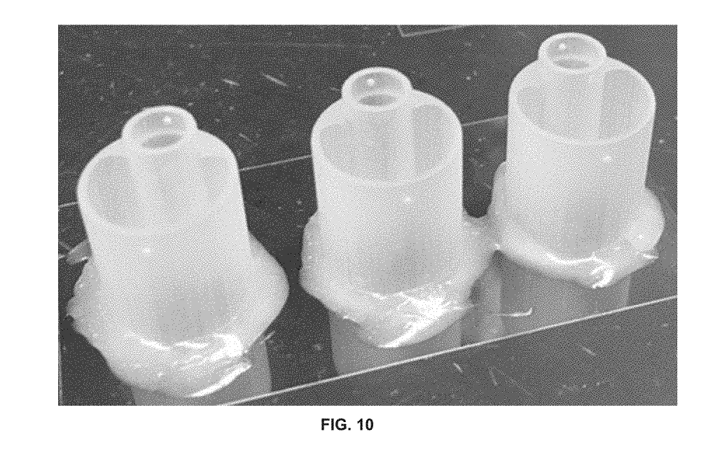

During the development of embodiments of the technology, a sample cell for electrophoretic delivery and concentration of analytes at a surface was produced using three-dimensional printing technology (see, e.g., FIG. 10).

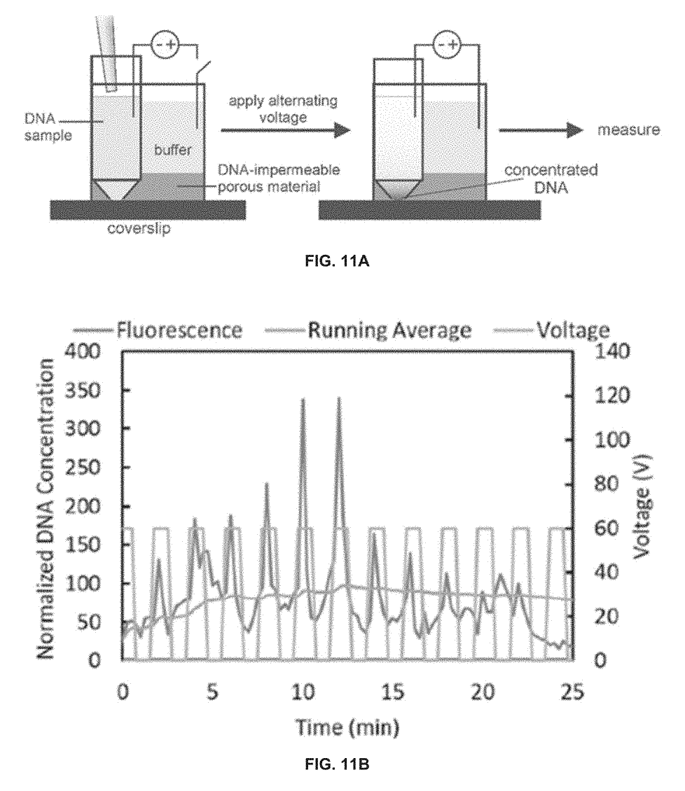

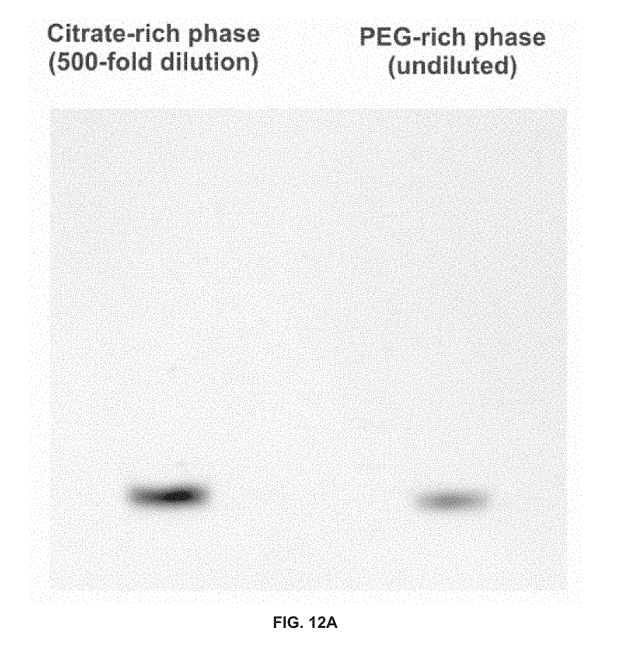

During the development of embodiments of the technology, a sample cell as described herein was constructed and used to concentrate DNA at a surface. Experiments were conducted to demonstrate the use of electrophoresis for concentrating fluorescently labeled DNA near an imaging surface. A solution of fluorescently labeled DNA was added to the inner chamber of an electrophoretic cell and an alternating voltage (between approximately 0 and 60-200 V) was applied with an oscillation period of approximately 1 minute to effect the migration of DNA toward the surface and thus to concentrate the DNA at the surface (see, e.g., FIG. 11A). A DNA-impermeable porous material (e.g., polyacrylamide that had been pre-blocked with dextran sulfate) prevents migration of DNA out of the well.

Fluorescence microscopy (total internal reflection fluorescence, TIRF) data were collected to quantify the DNA concentration at the surface under an alternating voltage (FIG. 11B). As indicated by the data collected, the time-averaged concentration of DNA was 50-fold to 100-fold higher at the surface than in the bulk solution in the presence of the electrophoresis voltage than in the absence of the electrophoresis voltage.

Furthermore, data collected during the experiments indicated that a 32% polyacrylamide matrix that was pre-blocked with 5-50 mg/mL dextran sulfate was sufficient to prevent migration of short DNA fragments (e.g., approximately 30 base pairs) out of the inner chamber.

In particular, polyacrylamide porous bases were prepared with and without pre-blocking with 5 mg/mL dextran sulfate. A visibly stained 34-nucleotide oligonucleotide was used for the experiments to assess electrophoresis of nucleic acid through the porous base. Photographs were taken of the porous bases comprising dextran sulfate and without dextran sulfate after electrophoresis. The photographs indicated that the 34-nucleotide oligonucleotide migrated out of the inner chamber and into the porous base (32% polyacrylamide) after electrophoresis, but that migration into the porous base was inhibited by polyacrylamide that was blocked by 5 mg/mL dextran sulfate. In some embodiments, the dextran sulfate was electrophoresed into the polyacrylamide from the inner chamber prior to introducing the DNA into the inner chamber.

While experiments tested the use of polyacrylamide blocked with dextran sulfate, the technology is not limited to this particular material. For instance, porous silica, regenerated cellulose, and other matrices may also be used that have a pore sizes that is small enough to exclude the analyte while being large and contiguous enough to permit flow of current. Further, embodiments include pre-blocking with a concentration of, e.g., 1, 2, 3, 4, 5, 6, 7, 8, 9, 10, 12, 15, 20, 22, 25, 30, 35, 40, 45, or 50 mg/mL the blocking agent (e.g., dextran sulfate).

In some embodiments, the use of alternating voltage causes migration of the analyte towards the surface and periodic reductions in the voltage allow the analyte to freely diffuse over the surface for efficient and homogeneous capture at the surface.

The technology provides high concentration factors (e.g., at least 50-fold to 100-fold concentration at the surface relative to the original solution). Further, the technology is compatible with a wide variety of analytes, including proteins and nucleic acids, provided that they are capable of being charged at a pH that is compatible with their stability. The technology finds use in concentrating analytes from very large volumes of solution (e.g., several milliliters) over a very small capture region (<1 mm) without requiring subsequent pipetting or manipulation of solutions.

### Example 13—Determining an ATPS Partition Coefficient for a Nucleic Acid

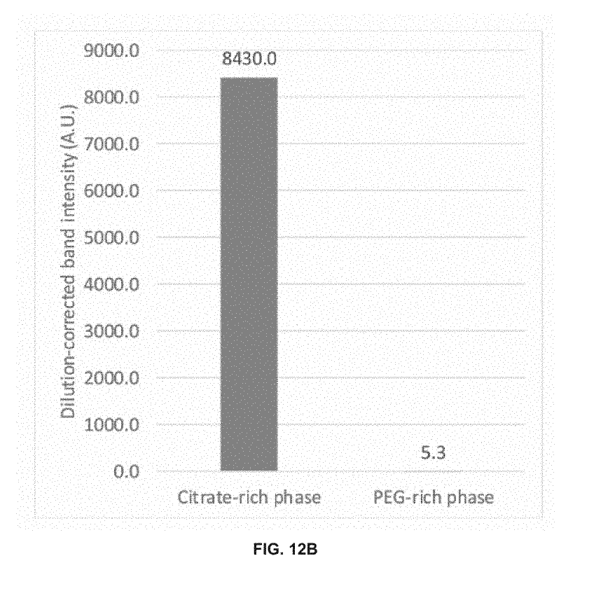

During the development of embodiments of the technology described herein, experiments were conducted to determine the partition coefficient for a 28-nucleotide single-stranded DNA molecule (e.g., GTCCAGGAGGCAGCTAATACTTGTTGCG; SEQ ID NO: 1) in a PEG/sodium citrate/sodium chloride aqueous two-phase system. An ATPS of PEG/sodium citrate/sodium chloride was produced from stock solutions and the 28-nucleotide single-stranded DNA molecule was added to the ATPS. Samples were taken from each phase after equilibration of the nucleic acid between the two phases. The samples were electrophoresed on a denaturing polyacrylamide gel to characterize partitioning of the DNA into the lower, citrate-rich phase and the upper, PEG-rich phase (FIG. 12A). The citrate-rich phase was diluted 500-fold relative to the PEG-rich phase prior to loading on the gel to permit simultaneous quantification of DNA in the two lanes on a single instrument without saturating the detector. Staining was performed with SYBR gold. The intensities of the bands on the gel were quantified by imaging the stained gel to determine the partition coefficient (FIG. 12B). The amount of DNA quantified in each phase was corrected for the 500-fold dilution factor of the citrate-rich phase. The apparent partition coefficient of this DNA oligonucleotide was determined to be 1590.

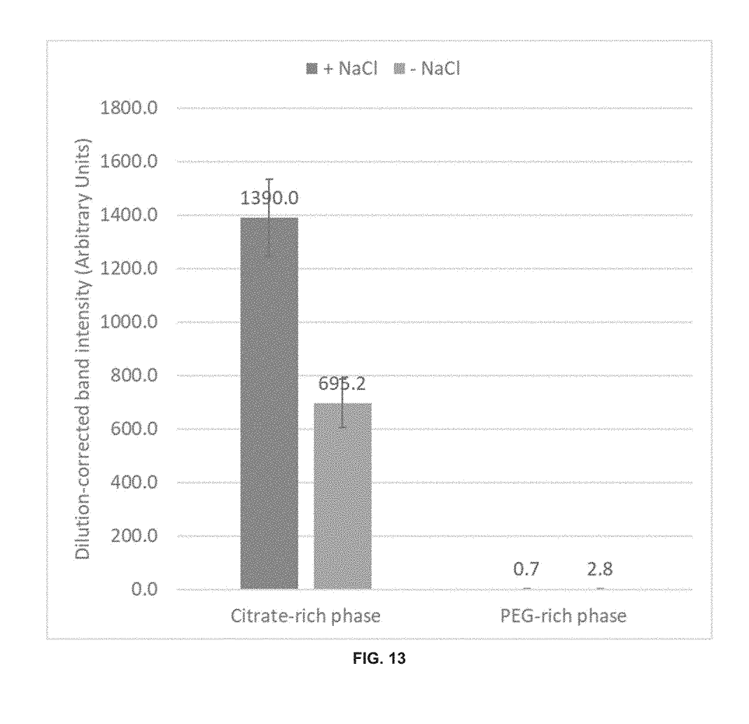

Next, experiments were conducted during the development of embodiments of the technology provided herein to measure the partitioning of a 28-nucleotide single-stranded DNA oligonucleotide (e.g., GTCCAGGAGGCAGCTAATACTTGTTGCG; SEQ ID NO: 1) in a PEG/sodium citrate ATPS in the presence or absence of 2.8% w/w NaCl. Partitioning between the two phases was quantified as in the experiment above, e.g., using by polyacrylamide gel electrophoresis followed by SYBR gold staining and densitometry to quantify bands on the gel. The citrate-rich phase was diluted 500-fold relative to the PEG-rich phase prior to loading on the gel to permit simultaneous quantification of DNA in the two lanes on a single instrument without saturating the detector. The calculated partition coefficients were 2030 and 249 in the presence and absence of NaCl, respectively (FIG. 13). Error bars represent +/−one standard deviation based on three independent replicates.

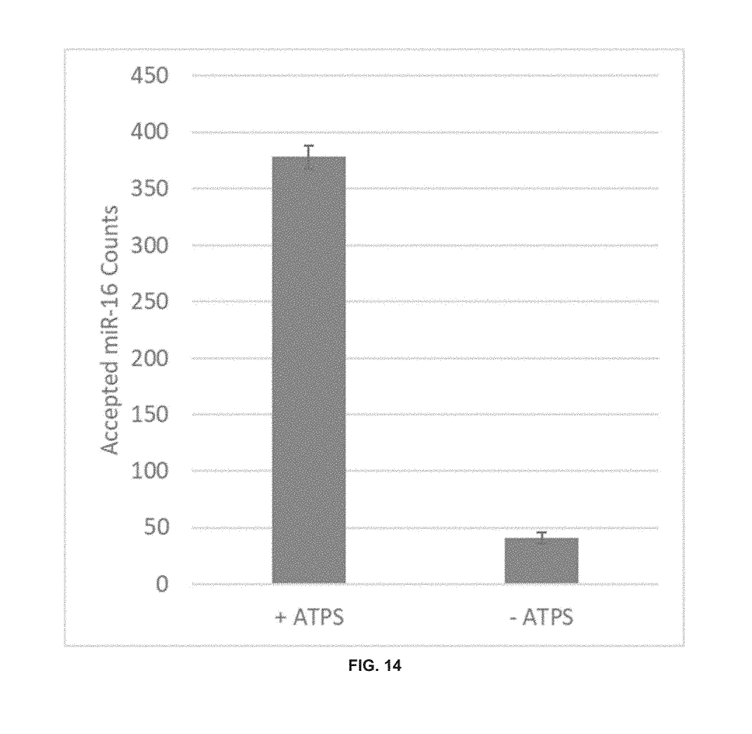

Finally, during the development of embodiments of the technology provided herein, experiments were conducted to test the use of ATPS enrichment of a nucleic acid to provide a sample for a SiMREPS assay. In particular, data were collected from a SiMREPS assay to detect the microRNA miR-16 with and without prior use of a PEG/sodium citrate/NaCl ATPS to enrich the sample for the microRNA. The apparent increase in sensitivity of SiMREPS with the use of this ATPS to enrich the nucleic acid was approximately 9-fold (FIG. 14). Error bars represent one standard deviation from two independent replicates.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method for analysis of an analyte on a substrate, the method comprising:
a) providing an aqueous two-phase system (ATPS) comprising an analyte;
b) concentrating the analyte into a first phase of the ATPS; and
c) contacting said first phase to a substrate.

2. The method of claim 1 further comprising separating said first phase from a second phase.

3. (canceled)

4. The method of claim 2 wherein the concentration of the analyte in the first phase is at least approximately 10 times the concentration of the analyte in the second phase.

5. The method of claim 2 wherein the volume of the second phase is at least approximately 10 times the volume of the first phase.

6. (canceled)

7. The method of claim 1 further comprising capturing the analyte by a capture probe immobilized on said substrate.

8. (canceled)

9. The method of claim 1 further comprising detecting the analyte on the substrate by analyzing the association and/or dissociation kinetics of a detectably labeled query probe and the analyte.

10. The method of claim 1 further comprising detecting the analyte on the substrate with single-molecule sensitivity.

11. (canceled)

12. The method of claim 1 wherein providing said ATPS comprises providing a mixture of a first composition and a second composition that form an ATPS upon adding an aqueous sample comprising said analyte to said mixture.

13-14. (canceled)

15. The method of claim 12 wherein said mixture is a solid, a neat mixture, or comprises solid and neat components.

16-19. (canceled)

20. The method of claim 1 wherein the analyte comprises a nucleic acid, a polypeptide, a metabolite, a lipid, a small molecule, or a carbohydrate.

21. The method of claim 1 further comprising providing a sample comprising said analyte.

22. The method of claim 21 wherein said sample is a biological sample.

23. (canceled)

24. The method of claim 1 wherein said first phase is contacted to a substrate over a restricted area of from 0.01 to 100 mm².

25-31. (canceled)

32. The method of claim 1 wherein said ATPS comprises polyethylene glycol (PEG) and citrate.

33. The method of claim 1 wherein said ATPS comprises PEG, citrate, and a salt.

34-35. (canceled)

36. The method of claim 21 further comprising heating said sample comprising said analyte and/or heating said ATPS comprising said analyte.

37-45. (canceled)

46. The method of claim 33 wherein the mass fraction of PEG in the mixture is greater than 30%, the mass fraction of citrate in the mixture is less than 2.5%, and the concentration of the salt is greater than 0.25 M.

47. The method of claim 33 wherein the molecular weight of said PEG is between 600 and 6000 Da.

48. The method of claim 33 wherein said citrate is present as citric acid and sodium citrate in a ratio to provide a pH between 5.5 and 8.5.

49. A system for detecting an analyte, the system comprising:
a) an aqueous two-phase system (ATPS) for concentrating an analyte in a first phase of the ATPS;
b) a substrate for capturing said analyte from said first phase of the ATPS; and
c) a system configured to detect said analyte on the substrate by analyzing the kinetics of association and dissociation of a detectably labeled query probe to said analyte.

50-109. (canceled)
